(19) United States
(12) Patent Application Publication
(10) Pub. No.: US 2004/0167183 A1
(43) Pub. Date: Aug. 26, 2004

(54) Phenethylamino sulfamic acids

(75) Inventors: Sean Rees Klopfenstein, Loveland, OH (US); Matthew Brian Maier, Cincinnati, OH (US); David Robert Jones, Milford, OH (US); Jeffrey Lyle Gray, Loveland, OH (US); Matthew Eugene Pokross, Loveland, OH (US); Kevin Gene Peters, Loveland, OH (US); Artem Gennady Evdokimov, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/633,743

(22) Filed: Aug. 4, 2003

Related U.S. Application Data
(60) Provisional application No. 60/455,977, filed on Mar. 18, 2003.
(60) Provisional application No. 60/448,749, filed on Feb. 20, 2003.

(51) Int. Cl. C07D 213/36; A61K 31/185; A61K 31/44

(52) U.S. Cl. 514/357; 514/553; 514/408; 562/37; 546/329

(57) ABSTRACT

Compounds of formula (I):

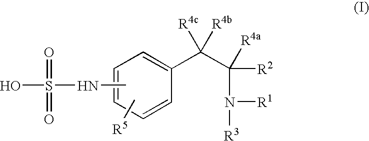

are effective in the treatment of protein tyrosine phosphatase (PTPase) mediated disorders such as diabetes.

### CROSS REFERENCE TO RELATED APPLICATIONS

This application claims priority under Title 35, United States Code 119(e) from Provisional Application Serial No. 60/455,977 filed Mar. 18, 2003; Provisional Application Serial No. 60/448,749 filed Feb. 20, 2003; and Provisional Application Serial No. 60/406,829, filed Aug. 29, 2002, incorporated herein by reference.

### FIELD OF THE INVENTION

The present invention relates to phenethylamino-sulfamic acids useful for the treatment of protein tyrosine phosphatase mediated disorders.

### BACKGROUND OF THE INVENTION

The regulation of protein tyrosine phosphorylation in vivo is mediated by the opposing actions of protein tyrosine kinases (PTKs) and protein tyrosine phosphatases (PTPases). The level of protein tyrosine phosphorylation of cellular proteins is determined by the balanced activities of PTKs, and PTPases (Hunter, Cell 80:225-236 (1995)). When there is an imbalance of these activities, a disease state may arise. By logical extension, modulation of the tyrosine kinase/phosphatase balance could be used to treat diseases resulting from such imbalances.

For example, the mechanism of insulin action depends critically upon the phosphorylation of tyrosine residues in several proteins in the insulin signaling cascade. Enzymes that dephosphorylate these proteins, i.e., PTPases, are important regulators of insulin action. Therefore, the use of PTPase inhibitors may therapeutically enhance insulin action.

PTPases are implicated in the insulin receptor signaling pathway. Insulin is an important regulator of different metabolic processes and plays a key role in the control of blood glucose. Defects related to its synthesis or signaling lead to diabetes mellitus. Binding of insulin to its receptor causes rapid (auto)phosphorylation of several tyrosine residues in the intracellular part of the insulin receptor (beta subunit). Three closely positioned tyrosine residues (the tyrosine1150 domain) must all be phosphorylated to obtain full activity of the insulin receptor tyrosine kinase (IRTK) which transmits the signal further downstream by tyrosine phosphorylation of other cellular substrates, including insulin receptor substrate-1 (IRS-1) (Wilden et al., J. Biol. Chem. 267: 16660-16668 (1992); Myers and White, Diabetes 42: 643-650 (1993); Lee and Pilch, Am. J. Physiol. 266: C319-C334 (1994); White et al., J. Biol. Chem. 263: 2969-2980 (1988)). The structural basis for the function of the tyrosine-triplet has been provided by recent X-ray crystallographic studies of IRTK that showed tyrosine-1150 to be autoinhibitory in its unphosphorylated state (Hubbard et al., Nature 372: 746-754 (1994)).

Several studies clearly indicate that the activity of the auto-phosphorylated IRTK can be reversed by dephosphorylation in vitro (reviewed in Goldstein, Receptor 3: 1-15 (1993); Mooney and Anderson, J. Biol. Chem. 264: 6850-6857 (1989)), with the tri-phosphorylated tyrosine-1150 domain being the most sensitive target for protein-tyrosine phosphatases (PTPases) as compared to the di- and mono- phosphorylated forms (King et al., Biochem. J. 275: 413-418 (1991)). It is, therefore, tempting to speculate that this tyrosine-triplet functions as a control switch of IRTK activity. Indeed, the IRTK appears to be tightly regulated by PTP-mediated dephosphorylation in vivo (Khan et al., J. Biol. Chem. 264: 12931-12940 (1989); Faure et al. J. Biol. Chem. 267: 11215-11221 (1992); Rothenberg et al, 1. Biol. Chem. 266: 8302-8311 (1991)). The intimate coupling of PTPases to the insulin signaling pathway is further evidenced by the finding that insulin differentially regulates PTPase activity in rat hepatoma cells (Meyerovitch et al, Biochemistry 31: 10338-10344 (1992)) and in livers from alloxan diabetic rats (Boylan et al., J. Clin. Invest. 90: 174-179 (1992)). Further, when the strong PTPase-inhibitor pervanadate is added to whole cells an almost full insulin response can be obtained in adipocytes (Fantus et al., Biochemistry 28: 8864-8871 (1989); Eriksson et al., Diabetologia 39: 235-242 (1995)) and skeletal muscle (Leighton et al., Biochem J. 276: 289-292 (1991)). In view of the forgoing, there is a need to identify inhibitors of PTPase that are useful in a method of treating insulin receptor tyrosine kinase mediated disorders.

In another example, acid phosphatases/PTPases may be involved in negative regulation of osteoblast proliferation. Therefore, the use of the PTPase inhibitors may therapeutically enhance osteoblast proliferation and thereby treat bone disorders.

The rate of bone formation is determined by the number and the activity of osteoblasts, which in turn are determined by the rate of proliferation and differentiation of osteoblast progenitor cells. Histomorphometric studies indicate that the osteoblast number is the primary determinant of the rate of bone formation in humans (Gruber et al., Mineral Electrolyte Metab. 12: 246-254 (1987); reviewed in Lau et al., Biochem. J. 257: 23-36 (1989)). Acid phosphatases/PTPases may be involved in negative regulation of osteoblast proliferation. Thus, fluoride, which has phosphatase inhibitory activity, has been found to increase spinal bone density in osteoporotics by increasing osteoblast proliferation (Lau et al., supra). Consistent with this observation, an osteoblastic acid phosphatase with PTPase activity was found to be highly sensitive to mitogenic concentrations of fluoride (Lau et al., J. Biol. Chem. 260: 46534660 (1985); Lau et al., J. Biol. Chem. 262:1389-1397 (1987); Lau et al., Adv. Protein Phosphatases 4: 165-198 (1987)). Interestingly, it was recently found that the level of membrane-bound PTPase activity was increased dramatically when the osteoblast-like cell line UMR 106.06 was grown on collagen type-I matrix compared to uncoated tissue culture plates. Since a significant increase in PTPase activity was observed in density-dependent growth arrested fibroblasts (Pallen and Tong, Proc. Natl. Acad. Sci. 88. 6996-7000 (1991)), it might be speculated that the increased PTPase activity directly inhibits cell growth. The mitogenic action of fluoride and other phosphatase inhibitors (molybdate and vanadate) may thus be explained by their inhibition of acid phosphatases/PTPases that negatively regulate the cell proliferation of osteoblasts. The complex nature of the involvement of PTPases in bone formation is further suggested by the recent identification of a novel parathyroid regulated, receptor-like PTPase, OST-PTP, expressed in bone and testis (Mauro et al. J. Biol. Chem. 269: 30659-30667 (1994)). OST-PTP is up-regulated following differentiation and matrix formation of primary osteoblasts and subsequently down-regulated in the osteoblasts which are actively mineralizing bone in culture. It may be hypothesized-that PTPase inhibitors may prevent differentiation via inhibition of OST-PTP or other PTPases thereby leading to continued proliferation. This would be in agreement with the above-mentioned effects of fluoride and the observation that the tyrosine phosphatase inhibitor orthovanadate appears to enhance osteoblast proliferation and matrix formation (Lau et al., Endocrinology 116: 2463-2468 (1988)). In addition, it was recently observed that vanadate, vanadyl and pervanadate all increased the growth of the osteoblast-like cell line UMRI06 (Cortizo et al., Mol. Cell. Biochem. 145: 97-102 (1995)).

In yet another example, the inhibition of acid phosphatases/PTPases may be involved in regulation of angiogenesis and tissue blood flow. Therefore, the use of PTPase inhibitors may be used to treat angiogenesis-mediated disorders.

Endothelial cells form the protective lining of blood vessels and respond to a variety of stimuli that modulate the form and function of the vasculature. Like the insulin receptor, the activity of endothelial PTKs is likely modulated by the action of endothelial PTPs. In support of this proposition, several PTPs have been shown to be expressed in endothelial cells (Fachinger et al. Oncogene 18:5948-5953 (1999); Huang et al. J. Biol. Chem. 274:38183-38188 (1999); Bianchi et al. Exp. Cell Res. 248:329-338 (1999); Gaits et al. Biochem. J. 311:97-103 (1995); Borges et al. Circ. Res. 79:570-580 (1996)). One of these phosphatases, HCPTPA, has been shown to interact with and attenuate the activation of a vascular endothelial growth factor (VEGF) receptor, VEGFR2, inhibiting VEGF-mediated downstream signaling and angiogenesis (Huang et al. J. Biol. Chem. 274:38183-38188 (1999)). Another phosphatase, HPTPbeta, associates with and attenuates the activation of the receptor for angiopoietin 1 (Ang1) and angiopoietin 2 (Ang2), Tie2, (Fachinger et al. Oncogene 18:5948-5953 (1999)). These studies indicate that targeting endothelial phosphatases will modulate the activation of endothelial PTKs and provide novel targets for therapeutic agents that modulate vascular form and function.

Abundant evidence demonstrates a role for multiple PTKs in the neovascularization of adult tissues. For example, inhibiting the action of VEGF or the angiopoietins inhibits tumor angiogenesis and limits tumor growth in animal models of cancer (Millauer et al. Cancer Res. 56:1615-1620 (1996); Dias et al. Proc. Natl. Acad. of Sci. 98:10857-10862 (2001); Lin et al. Proc. Natl. Acad. of Sci. 95:8829-8834 (1998)). Conversely, administration of exogenous VEGF and/or Ang1 enhances the development of the collateral circulation and improves blood flow to ischemic tissue in animal models of occlusive cardiovascular disease (Witzenbichler et al. Am. J. of Pathol. 153:381-394 (1998); Pearlman et al. Nature Medicine 10: 1085-1089 (1995); Banai et al. Circulation 89:2183-2189 (1994); Shyu et al. 98:2081-2087 (1998); Chae et al. Arteriosclero. Thromb. Vasc. Biol. 20:2573-2578 (2000)). Taken together, these studies not only demonstrate a role for PTKs in neovascularization, but they also demonstrate that modulating the function of endothelial PTKs provides a novel therapeutic approach to modulation of angiogenesis and tissue blood flow in a broad range of diseases. Diseases in which enhanced vascular development would be beneficial include, but are not limited to, occlusive atherosclerotic cardiovascular disease, coronary artery disease, peripheral vascular disease, cerebrovascular disease (stroke), Berger's disease, diabetic vasculopathy and traumatic vascular damage. Diseases in which inhibition of neovascularization would be beneficial, include but are not limited to, cancer, arthritis, diabetic retinopathy, macular degeneration, psoriasis and endometriosis. In view of the foregoing, there is a need to identify inhibitors of PTPase that are useful in a method of treating angiogenesis-mediated disorders.

In yet another example, the inhibition of acid phosphatases/PTPases may be involved in regulation of vascular tone. In addition to neovascularization and vascular remodeling, activation of PTKs can influence certain parameters of vascular function. Therefore, the use of PTPase inhibitors may be used to treat vascular tone mediated disorders.

Vascular tone is regulated by the endothelium and the endothelial factors that regulate vascular tone can be modulated by endothelial PTK signaling. Bolus infusion of VEGF induces a hypotensive response that is driven in part by VEGF-mediated activation of nitric oxide (NO) synthase and subsequent production by the endothelium of the potent vasorelaxant, nitric oxide (Hariawala et al. J Surgical Res. 63:77-82 (1996); Ogasawara et al. Hypertension 39:815-820 (2002)). Administration of fibroblast growth factor (FGF) has similar effects as VEGF on blood pressure that may also be mediated by enhanced endothelial nitric oxide production (Garcia-Calvo et al. Proc. Natl. Acad. Sci. 93:11996-12001 (1996); Wu et al. Am. J. Physiol. 271:H1087-1093 (1996); Cuevas et al. Science 254:1208-1210 (1991)). Thus, modulating PTK activation and downstream signaling provides a novel therapeutic approach to treating diseases characterized by alterations of vascular tone. Diseases which would benefit from decreased vascular tone include primary essential hypertension, secondary hypertension (i.e. renovascular or endocrine disorder mediated), pulmonary hypertension and portal hypertension. In view of the foregoing, there is a need to identify inhibitors of PTPase that are useful in a method of treating vascular tone mediated disorders.

In yet another example, the inhibition of acid phosphatases/PTPases may be involved in regulation of vascular permeability. Activation of endothelial PTKs has been shown to influence vascular permeability. Therefore, the use of PTPase inhibitors may be used to treat vascular permeability mediated disorders.

VEGF was originally isolated as a factor that increased vascular permeability (Senger et al. Cancer Metastasis Rev. 12:303-324 (1993)). VEGF induced vascular permeability may be induced by the same high affinity receptor PTKs that mediate the other actions of VEGF i.e. angiogenesis and vasorelaxation (Gomez et al. Endocrinology 143:43394348 (2002); Murohara et al. Circulation 97:99-107 (1998)). In contrast to the permeabilizing effects of VEGF, Ang1 via its high affinity receptor, Tie2, blocks increases in vascular permeability by a variety of agents including VEGF (Thurston et al. Science 286:2511-2514 (1999); Thurston et al. Nature Medicine 6:460463 (2000)). These data demonstrate that activation and signaling by endothelial PTKs can either enhance or decrease vascular permeability and that approaches to specifically modulate endothelial PTK activation and signaling offers a novel therapeutic approach to pathologic states characterized by alterations in vessel leakiness. Diseases in which reducing vascular permeability would be beneficial include, but are not limited to, stroke, septic shock, burns, RDS (respiratory distress syndrome) and congestive heart failure. In view of the foregoing, there is a need to identify inhibitors of PTPase that are useful in a method of treating vascular permeability disorders.

In yet another example, the inhibition of acid phosphatases/PTPases may be involved in regulation of VEGF and thus the use of PTPase inhibitors may be used to treat VEGF-mediated disorders.

In addition to effecting the form and function of the vascular system directly, modulating the activity of signaling by endothelial PTKs has been shown to have indirect beneficial effects on other tissues. For example, decreasing the expression of VEGF in the myocardium results in the development of an ischemic cardiomyopathy (Carmeliet et al. Nat. Med. 5:495-502 (1999)). Conversely, exogenous delivery of VEGF improves cardiac performance in animal models of heart failure and myocardial infarction (Suzuki et al. Circulation 104[suppl I]:I-207-1-212 (2001); Leotta et al. J. Thorac. Cardiovasc. Surg. 123:1101-1113 (2002)). Increasing evidence indicates that VEGF can directly and indirectly effect the peripheral nervous system (Carmeliet et al. Semin. Cell. Dev. Biol. 13:39-53 (2002)). Delivery of exogenous VEGF can reverse experimental diabetic neuropathy and early evidence from a small clinical trial suggests that this approach could be extended to patients with diabetic neuropathy (Schratzberger et al. J. Clin. Invest. 107:1083-1092 (2001); Veves et al. J. Clin. Invest. 107:1215-1218 (2001); Hum. Gene Ther. 12:1593-1594 (2001)). Strong evidence now indicates that VEGF plays a crucial role in bone development and delivery of exogenous VEGF enhances bone healing (Zelzer et al. Development 129:1893-1904 (2002); Maes et al. Mech Dev 111:61-73 (2002); Gerber et al. Nat. Med. 5:623-628 (1999); Peng et al. J. Clin. Invest. 110:751-759 (2002); Street et al. Proc. Natl. Acad. Sci. 99:9656-9661 (2002)). In addition to bone fracture healing, recent evidence also suggests that enhancing VEGF signaling also accelerates healing of skin wounds even in an animal model of diabetes where wound healing is delayed (Di Peppe et al. Gene Ther. 9:1271-1277 (2002)). Finally, VEGF and VEGF receptors are expressed in hair follicles and transgenic delivery of VEGF in hair follicles enhances hair growth whereas inhibition of VEGF action attenuates hair growth (Yano et al. J. Clin. Invest. 107:409417 (2001)). Thus enhancing the activation of endothelial PTKs, and VEGF receptors in particular, represents a novel therapeutic approach for heart failure, myocardial infarction, diabetic and ischemic neuropathy (and perhaps other neuropathic conditions), osteoporosis, bone fracture healing, wound healing and hair loss. In view of the foregoing, there is a need to identify inhibitors of PTPase that are useful in a method of treating VEGF-mediated disorders.

Therefore in view of the forgoing, there is a need to identify inhibitors of PTPase that are useful for the treatment of PTPase mediated disorders.

### SUMMARY OF THE INVENTION

The present invention meets the aforementioned need by identifying and providing Phenethylamino sulfamic acids that are effective in the treating PTPase mediated disorders.

The first aspect of the present invention relates to compounds, including all enantiomeric and diasteriomeric forms and pharmaceutically acceptable salts thereof, having the formula (I):

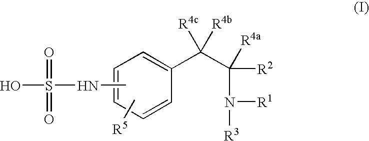

wherein:

A) R¹ is L¹—[C(R^{6a}R^{6b})]ₘR⁷, wherein:

a) L¹ is selected from the group consisting of covalent bond, —O—, —S—, —N—, —CO₂]—CO—, —OCO₂—, —SO—, —SO₂—, —CSN(R⁸)—, —CON(R⁸)O—, —CON(R⁸)—, —OCON(R⁸)—; wherein R⁸ is hydrogen or substituted or unsubstituted C₁-C₅ alkyl;

b) R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, and mixtures thereof; wherein each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms;

c) m is an index selected from 0 to 5;

d) R⁷ is selected from the group consisting of nil, hydrogen, substituted or unsubstituted C₁-C₁₀ alkyl, substituted or unsubstituted C₁-C₁₀ heteroalkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or

e) R⁷ and a R⁹ can be taken together to form a substituted or unsubstituted carbocyclic or heterocyclic ring comprising from 3 to 7 atoms;

B) R² is —(CH₂)ⱼ-L²-[C(R^{11a}R^{11b})]_{g}R², wherein:

a) j is an index selected from 0 to 5;

b) L² is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—; wherein R¹⁰ is hydrogen or substituted or unsubstituted C₁-C₅ alkyl;

c) R^{11a} and R^{11b} are each independently selected from the group consisting of hydrogen, —OR¹³, —N(R¹³)₂, —CO₂R¹³, —CON(R¹³)₂, —NHCOR¹³, —NHCO₂R¹³, ═NR¹³, —R¹³, and mixtures thereof; wherein each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms;

d) g is an index selected from 0 to 5;

e) R¹² is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or

f) R¹² and a R¹³ can be taken together to form a substituted or unsubstituted carbocyclic or heterocyclic ring comprising from 3 to 7 atoms;

C) R³ is —(CH₂)ₙ-L³-R⁶, wherein:

a) n is an index selected from 0 to 5;

b) L³ is selected from covalent bond, —O—, —S—, —N—, —CO₂—, —CO—, —OCO₂—, —SO—, —SO₂—, —CSNH—, —CONH—, —OCONH—;

c) R 6 is selected from the group consisting of hydrogen, substituted or unsubstituted C₁-C₁₀ alkyl, substituted or unsubstituted C₁-C₁₀ heteroalkyl substituted or unsubstituted aryl or alkylenearyl, substituted or unsubstituted heterocyclyl, substituted or unsubstituted heteroaryl or alkyleneheteroaryl;

D) R^{4a}, R^{4b}, R^{4c} and R⁵ are each independently selected from hydrogen or substituted unit; or

E) R² and R^{4a}, R^{4a} and R^{4b}, R¹ and R², or R¹ and R³ can be taken together to form a substituted or unsubstituted carbocyclic or heterocyclic ring comprising from 3 to 7 atoms.

Another aspect of the invention provides a pharmaceutical composition comprising a safe and effective amount of an above-identified compound and a pharmaceutically acceptable carrier.

Another aspect of the invention provides a method of administering to a subject in need thereof a safe and effective amount of an above-identified compound for the treatment of a PTPase mediated disorder.

These and other objects, features, and advantages will become apparent to those of ordinary skill in the art from a reading of the following detailed description and the appended claims.

### DETAILED DESCRIPTION OF THE INVENTION

I. Terms and Definitions

The following is a list of definition for terms used herein:

The term “hydrocarbyl,” as defined herein, means any organic unit or moiety which is comprised of carbon atoms and hydrogen atoms. Included within the term hydrocarbyl are the heterocycles which are described herein below. Examples of various unsubstituted non-heterocyclic hydrocarbyl units include pentyl, 3-ethyloctanyl, 1,3-dimethylphenyl, cyclohexyl, cis-3-hexyl, 7,7-dimethylbicyclo[2.2.1]-heptan-1-yl, and naphth-2-yl.

Included within the definition of “hydrocarbyl” are the aromatic (aryl) and non-aromatic carbocyclic rings, non-limiting examples of which include cyclopropyl, cyclobutanyl, cyclopentanyl, cyclohexane, cyclohexenyl, cycloheptanyl, bicyclo-[0.1.1]-butanyl, bicyclo-[0.1.2]-pentanyl, bicyclo-[0.1.3]-hexanyl (thujanyl), bicyclo-[0.2.2]-hexanyl, bicyclo-[0.1.4]-heptanyl (caranyl), bicyclo-[2.2.1]-heptanyl (norboranyl), bicyclo-[0.2.4]-octanyl (caryophyllenyl), spiropentanyl, diclyclopentanespiranyl, decalinyl, phenyl, benzyl, naphthyl, indenyl, 2H-indenyl, azulenyl, phenanthryl, anthryl, fluorenyl, acenaphthylenyl, 1,2,3,4-tetrahydronaphthalenyl, and the like.

The term “heterocycle” includes both aromatic (heteroaryl) and non-aromatic heterocyclic rings non-limiting examples of which include: pyrrolyl, 2H-pyrrolyl, 3H-pyrrolyl, pyrazolyl, 2H-imidazolyl, 1,2,3-triazolyl, 1,2,4-triazolyl, isoxazolyl, oxazoyl, 1,2,4-oxadiazolyl, 2H-pyranyl, 4H-pyranyl, 2H-pyran-2-one-yl, pyridinyl, pyridazinyl, pyrimidinyl, pyrazinyl, piperazinyl, s-triazinyl, 4H-1,2-oxazinyl, 2H-1,3-oxazinyl, 1,4-oxazinyl, morpholinyl, azepinyl, oxepinyl, 4H-1,2-diazepinyl, indenyl 2H-indenyl, benzofuranyl, isobenzofuranyl, indolyl, 3H-indolyl, 1H-indolyl, benzoxazolyl, 2H-1-benzopyranyl, quinolinyl, isoquinolinyl, quinazolinyl, 2H-1,4-benzoxazinyl, pyrrolidinyl, pyrrolinyl, quinoxalinyl, furanyl, thiophenyl, benzimidazolyl, and the like each of which can be substituted or unsubstituted.

An example of a unit defined by the term “alkylenearyl” is a benzyl unit having the formula:

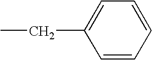

whereas an example of a unit defined by the term “alkyleneheteroaryl” is a 2-picolyl unit having the formula:

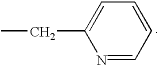

The term “substituted” is used throughout the specification. The term “substituted” is defined herein as “encompassing moieties or units which can replace a hydrogen atom, two hydrogen atoms, or three hydrogen atoms of a hydrocarbyl moiety. Also substituted can include replacement of hydrogen atoms on two adjacent carbons to form a new moiety or unit.” For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. An epoxide unit is an example of a substituted unit which requires replacement of a hydrogen atom on adjacent carbons. The term substituted is used throughout the present specification to indicate that a hydrocarbyl moiety, inter alia, aromatic ring, alkyl chain, can have one or more of the hydrogen atoms replaced by a substituent. When a moiety is described as “substituted” any number of the hydrogen atoms may be replaced. For example, 4-hydroxyphenyl is a “substituted aromatic carbocyclic ring”, (N,N-dimethyl-5-amino)octanyl is a “substituted C₈ alkyl unit, 3-guanidinopropyl is a “substituted C₃ alkyl unit,” and 2-carboxypyridinyl is a “substituted heteroaryl unit.” The following are non-limiting examples of substituted units which can serve as a replacement for hydrogen atoms when a hydrocarbyl unit is described as “substituted.”

i) —[C(R¹⁵)₂]ₚ(CH═CH)_{q}R¹⁵;

ii) —[C(R¹⁵)₂]ₚC(Z)R¹⁵;

iii) —[C(R¹⁵)₂]ₚC(Z)₂R¹⁵;

iv) —[C(R¹⁵)₂]ₚC(Z)CH═CH₂;

v) —[C(R¹⁵)₂]ₚC(Z)N(R¹⁵)₂;

vi) —[C(R¹⁵)₂]ₚC(Z)NR¹⁵N(R¹⁵)₂;

vii) —[C(R¹⁵)₂]ₚCN;

viii) —[C(R¹⁵)₂]ₚCNO;

ix) —[C(R¹⁵)₂]ₚCF₃, —[C(R¹⁵)₂]ₚCCl₃, —[C(R¹⁵)₂]ₚCBr₃;

x) —[C(R¹⁵)₂]ₚN(R¹⁵)₂;

xi) —[C(R¹⁵)₂]ₚNR¹⁵CN;

xii) —[C(R⁵)₂]ₚNR¹⁵C(Z)R¹⁵;

xiii) —[C(R¹⁵)₂]ₚNR¹⁵C(Z)N(R¹⁵)₂;

xiv) —[C(R¹⁵)₂]ₚNHN(R¹⁵)₂;

xv) —[C(R¹⁵)₂]ₚNHOR¹⁵;

xvi) —[C(R¹⁵)₂]ₚNHSO₃M;

xvi) —[C(R¹⁵)₂]ₚNCS;

xvii) —[C(R¹⁵)₂]ₚNO₂;

xviii) —[C(R¹⁵)₂]ₚOR¹⁵;

xix) —[C(R¹⁵)₂]ₚOCN;

xx) —[C(R¹⁵)₂]ₚOCF₃, —[C(R¹⁵)₂]ₚOCCl₃, —[C(R¹⁵)₂]ₚOCBr₃;

xxi) —[C(R¹⁵)₂]ₚF, —[C(R¹⁵)₂]ₚCl, —[C(R¹⁵)₂]pBr, —[C(R¹⁵)₂]ₚI, and mixtures thereof;

xxii) —[C(R¹⁵)₂]ₚSCN;

xxiii) —[C(R¹⁵)₂]ₚSO₃M;

xxiv) —[C(R¹⁵)₂]ₚOSO₃M;

xxv) —[C(R¹⁵)₂]ₚSO₂N(R¹⁵)₂;

xxvi) —[C(R¹⁵)₂]ₚSO₂NH(R¹⁵);

xxvii) —[C(R¹⁵)₂]ₚSO₂NHCOR¹⁵;

xxviii) —[C(R¹⁵)₂]ₚSO₂NHCOOR¹⁵;

xxvi) —[C(R¹⁵)₂]ₚSO₂R¹⁵;

xxvii) —[C(R¹⁵)₂]ₚP(O)H₂;

xxviii) —[C(R¹⁵)₂]ₚPO₂;

xxix) —[C(R¹⁵)₂]ₚP(O)(OH)₂;

xxx) —[C(R¹⁵)₂]ₚCO₂M;

xxxi) —[C(R¹⁵)₂]ₚSR¹⁵;

xxxii) and mixtures thereof;

wherein R¹⁵ is hydrogen, substituted or unsubstituted C₁-C₂₀ linear, branched, or cyclic alkyl, C₆C₂₋₀ aryl, C₇-C₂₀ alkylenearyl, and mixtures thereof; M is hydrogen, or a salt forming cation; Z is ═O, ═S, ═NR¹⁵, and mixtures thereof; p is from 0 to 12; q is from 0 to 12. Suitable salt forming cations include, sodium, lithium, potassium, calcium, magnesium, ammonium, and the like.

II. Compounds

A first aspect of the present invention relates to compounds having the formula:

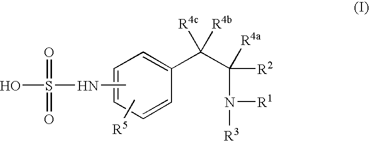

wherein R¹, R², R³, R^{4a}, R^{4b}, R^{4c}, R⁵ are previously defined.

The following are the various aspects of non-limiting preferred moieties; however, the formulator is not limited to the herein exemplified iterations and examples.

A) The single sulfamic acid moiety (HOSO₂NH—) of formula (I) is attached at the 4, 5, 6, 7, or 8-position of the phenethylamino scaffold. Conventional numbering is herein presented:

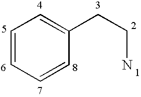

In one embodiment, the sulfamic acid moiety is at the 5 or 6-position of the scaffold. In another embodiment, the sulfamic acid moiety is at the 6-position of the scaffold.

B) R¹ is -L¹-[C(R^{6a}R^{6b})]ₘR⁷. L¹ is selected from the group consisting of covalent bond, —O—, —S—, —N—, —CO₂—, —CO—, —OCO₂—, —SO—, —SO₂—, —CSN(R⁸)—, —CON(R⁸)—, —CON(R⁸)O—, and —OCON(R⁸)—; wherein R⁸ is hydrogen or substituted or unsubstituted C₁-C₅ alkyl. In one embodiment, L¹ is selected from the group consisting of covalent bond —CO₂—, —CO—, —SO₂—, and —CON(R⁸)—. In another embodiment, L¹ is selected from the group consisting of —CO₂—, —CO—, —SO₂—, and —CON(R⁸)—. In another embodiment, L¹ is —CONH—. In another embodiment, L¹ is a covalent bond.

R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, andmixtures thereof; wherein each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms.

In one embodiment, R^{6a} and R^{6b} are each hydrogen or —NHCO₂R⁸. In another embodiment, R^{6a} and R^{6b} are each hydrogen. In another embodiment, R^{6a} and R^{6b} are each unsubstituted aryl. In another embodiment, R^{6a} or R^{6b} is —NHCOR⁹. In another embodiment, R^{6a} or R^{6b} is —NHCOR⁹ wherein R⁹ is substituted alkyl. In another embodiment, R^{6a} is unsubstituted aryl and R^{6b} is substituted alkyl wherein the substituted unit is least one selected from either carboxylate or carbonyl.

Index m is selected from 0 to 5. In one embodiment, index m is 0. In another embodiment, index m is 1.

R⁷ is selected from the group consisting of nil, hydrogen, substituted or unsubstituted C₁C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl. In one embodiment, R⁷ is selected from the group consisting of: substituted or unsubstituted C₁-C₁₀ alkyl; substituted or unsubstituted aryl; substituted or unsubstituted heteroaryl; substituted or unsubstituted alkylenearyl; and substituted or unsubstituted alkyleneheteroaryl.

In one embodiment, R⁷ is substituted or unsubstituted C₁-C₁₀ alkyl. In another embodiment, R⁷ is unsubstituted C₁-C₁₀ alkyl wherein the alkyl is branched. In another embodiment, R⁷ is tert-butyl. In another embodiment, R⁷ is selected from the group consisting of —CH₃, —CH₂CH₃, —CH₂CH₂CH₃, —(CH2)₃, —(CH₂)₄CH₃, —(CH₂)₅CH₃, —C(CH₃)₅, —CH₂C(CH₃)₃, —CH₂C(CH₃)₂CH₂CH₃, —C(CH₃)₂CH₂CH₃, —CH(CH₃)CH₂CH₃, and —CH₂CH(CH₂CH₃)₂.

In one embodiment, R⁷ is substituted C₁-C₁₀ alkyl, wherein the substituted unit is [C(R¹⁵)₂]ₚC(Z)N(R¹⁵)₂. In another embodiment, R⁷ is substituted C₁-C₅ alkyl, wherein the substituted unit is —CONH₂.

In one embodiment, R⁷ is heteroalkyl. In one embodiment, R⁷ is a C₁-C₅ heteroalkyl comprising at least one heteroatom. In another embodiment, R⁷ is a C₁-C₅ heteroalkyl wherein the heteroatom is at least S.

In one embodiment, R⁷ is substituted or unsubstituted alkylenearyl. In another embodiment, R⁷ is substituted or unsubstituted C₇-C₁₂ alkylenearyl. In another embodiment, R⁷ is selected from the group consisting of —CH₂(C₆H₅), —CH₂CH₂(C₆H₅), —(CH₂)₃(C₆H₅), —(CH₂)₄(C₆H₅), —CH₂(C₁₀H₇), —CH₂CH₂(C₁₀H₇), —(CH₂)₃(C₁₀H₇), —(CH₂)₄(C₁₀H₇), and mixtures thereof. In another embodiment, R⁷ is substituted alkylenearyl; wherein the substituted unit is selected from the group consisting of —[C(R¹⁵)₂]ₚSO₂N(R¹⁵)₂, —[C(R¹⁵)₂]ₚSO₂NH(R¹⁵), —[C(R¹⁵)₂]ₚSO₂NHCOR⁵; —[C(R⁵)₂]ₚSO₂NHCOR⁵, and mixtures thereof; wherein still another embodiment the substituted unit is selected from the group consisting of —SO₂NH₂, —SO₂NHCOOCH₃, —SO₂NHCOOCH₂CH₃, —SO₂NHCOCH₃, —SO₂NHCOCH₂CH₃, —SO₂NHCOC(CH₃)₃, —SO₂NH(C₆H₅), —SO₂NHCO(C₆H₅), —SO₂NHCOCH₂(C₆H₅), —SO₂NHCOCH₂CH₂(C₆H₅), and mixtures thereof.

In one embodiment, R⁷ is substituted or unsubstituted aryl. In another embodiment, R⁷ is substituted aryl, wherein the substituted unit is least one selected from the group consisting of —CH₃, —CH₂CH₃, —CH₂CH₂CH₃, —C(CH₃)₃, —OCH₃, —OCH₂CH₃, and mixtures thereof. In another embodiment, R⁷ is substituted aryl, wherein the substituted unit is a halogen.

In one embodiment, R¹ is a guanidine or amidine moiety. In another embodiment, L¹ is covalent bond, R⁷ is nil; index m is 1; and R^{6a} is ═NR⁹, and R^{6b} is —R⁹ or —N(R⁹)₂, preferably —N(R⁹)₂ is —NHR⁹. In another embodiment, L is covalent bond, R⁷ is nil; index m is 1; and R^{6a} is ═NR⁹, and R^{6b} is either —R⁹ or —N(R⁹)₂; wherein two R⁹ units can be taken together to form a substituted or unsubstituted carbocyclic or heterocyclic ring comprising from 3 to 7 atoms.

B) R² is —(CH₂)ⱼ-L 2-[C(R^{11a}R^{11b})]_{g}R 2. Index j is selected from 0 to 5. L² is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—. In turn, R¹⁰ is selected from hydrogen or substituted or unsubstituted C₁-C₅ alkyl. R^{11a} and R^{11b} are each independently selected from the group consisting of hydrogen, —OR³, —N(R¹³)₂, —CO₂R³, —CON(R¹³)₂, —NHCOR³, —NHCO₂R¹³, ═NR¹³, —R¹³, and mixtures thereof. Each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms. Index g is selected from 0 to 5. R¹² is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl. Alternatively, R¹² and a R¹³ can be taken together to form a substituted or unsubstituted carbocyclic or heterocyclic ring comprising from 3 to 7 atoms.

In one embodiment, index j is 0. In another embodiment, index j is 1.

In one embodiment, L² is selected from the group consisting of covalent bond, —CONR₈—, —CONH—, —CON(CH₃)—, —CO₂—, —CO—, and mixtures thereof. In another embodiment, L² is selected from the group consisting of CONR₈—, —CONH—, and mixtures thereof.

In one embodiment, R^{11a} and R^{11b} are both hydrogen.

In one embodiment, R^{11a} or R^{11b} is substituted or unsubstituted C₇-C₂₀ alkylenearyl. In another embodiment, R^{11a} or R^{11b} is unsubstituted C₇-C₁₀ alkylenearyl. In another embodiment, R^{11a} or R^{11b} is selected from the group consisting of —CH₂(C₆H₅), —CH₂CH₂(C₆H₅), —(CH₂)₃(C₆H₅), —(CH₂)₄(C₆H₅), —CH₂(C₁₀H₇), —CH₂CH₂(C₁₀H₇), —(CH₂)₃(C₁₀H₇), —(CH₂)₄(C₁₀H₇), and mixtures thereof. In one embodiment, R^{11a} or R^{11b} is —CON(R¹³)₂.

In one embodiment index g is selected from 0 and 1. In another embodiment, index g is 0.

In one embodiment, R¹² is substituted or unsubstituted C₁-C₁₀ alkyl. In another embodiment, R¹² is selected from the group consisting of —CH₃, —CH₂CH₃, —CH₂CH₂CH₃, —(CH₂)₃, —(CH₂)₄CH₃, —(CH₂)₅CH₃, —C(CH₃)₅, —CH₂C(CH₃)₃, —CH₂C(CH₃)₂CH₂CH₃, —C(CH₃)₂CH₂CH₃, —CH(CH₃)CH₂CH₃, —CH₂CH(CH₂CH₃)₂, and mixtures thereof.

In one embodiment, R¹² is a substituted or unsubstituted heterocyclic ring comprising from 3 to 7 atoms. In another embodiment, R¹² is substituted or unsubstituted morpholine. In another embodiment, R¹² is substituted or unsubstituted pyrazole.

In one embodiment, R¹² is a C₁-C₅ heteroalkyl comprising at least one heteroatom. In another embodiment, R¹² is a C₁-C₅ heteroalkyl wherein the heteroatom is O or S.

In one embodiment, R¹² is hydrogen.

C)R³ is —(CH₂)ₙ-L³-R¹⁶. Index n is selected from 0 to 5. L³ is selected from covalent bond, —O—, —S—, —N—, —CO₂—, —CO—, —OCO₂—, —SO—, —SO₂—, —CSNH—, —CONH—, and —OCONH—. R¹⁶ is selected from the group consisting of hydrogen, substituted or unsubstituted C₁-C₁₀ alkyl, substituted or unsubstituted C₁-C₁₀ heteroalkyl substituted or unsubstituted aryl or alkylenearyl, substituted or unsubstituted heterocyclyl, substituted or unsubstituted heteroaryl or alkyleneheteroaryl.

In one embodiment, index n is either 1 or 0.

In one embodiment, L³ is selected from the group consisting of hydrogen, covalent bond, —CO—, and —CO₂—.

In one embodiment, R¹⁶ is unsubstituted C1-C10 alkyl. In another embodiment, R¹⁶ is selected from —CH₃, —CH₂CH₃, —CH₂CH₂CH₃, —(CH₂)₃, —(CH₂)₄CH₃, —(CH₂)₅CH₃, —C(CH₃)₅, —CH₂C(CH₃)₃, —CH₂C(CH₃)₂CH₂CH₃, —C(CH₃)₂CH₂CH₃, —CH(CH₃)CH₂CH₃, and —CH₂CH(CH₂CH₃)₂. In another embodiment R¹⁶ is substituted C1-C5 alkyl wherein the alkyl is substituted with at least a carboxylate. In another embodiment, R¹⁶ is a substituted or unsubstituted alkyl and wherein said alkyl is an alkenyl (i.e., having at least one olefinic double bond).

In one embodiment, R¹⁶ is selected from substituted or unsubstituted C₇-C₁₀ alkylenearyl. R^{11a} or R^{11b} is —CH₂(C₆H₅), —CH₂CH₂(C₆H₅), —(CH₂)₃(C₆H₅), —(CH₂)₄(C₆H₅), —CH₂(C₁₀H₇), —CH₂CH₂(C₁₀H₇), —(CH₂)₃(C₁₀H₇), and —(CH₂)₄(C₁₀H₇). In another embodiment, R¹⁶ is a substituted alkylenearyl and wherein the substituted unit is at least —[C(R¹⁵)₂],NHSO₃M.

D) R^{4a}, R^{4b}, R^{4c} and R are each independently selected from hydrogen or substituted unit.

E) R² and R^{4a}, or R^{4a} and R^{4b} can be taken together to form a substituted or unsubstituted carbocyclic or heterocyclic ring comprising from 3 to 7 atoms.

III. Compound Preparation

The compounds of the invention can be prepared using a variety of procedures. The starting materials used in preparing the compounds of the invention are known, made by known methods, or are commercially available. Particularly preferred syntheses are described in the following general reaction schemes. (The R groups used to illustrate the reaction schemes do not necessarily correlate to the respective R groups used to describe the various aspects of the Formula (I) compounds. That is, for example, R₁ in Formula (I) does not represent the same moiety as R₁ here.) Specific examples for making the compounds of the present invention are set forth in Section VI, below.

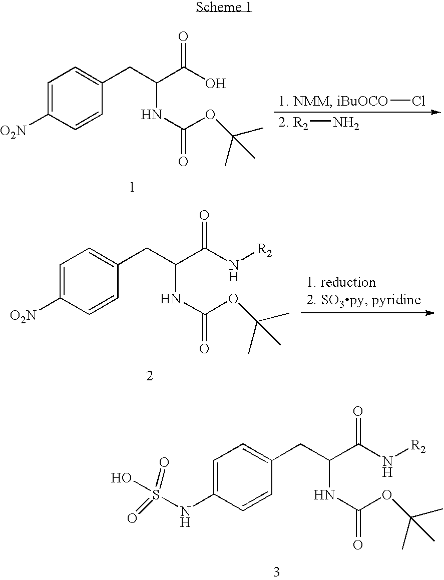

In reference to Scheme 1, starting compound t-butoxycarbonyl (Boc) protected 4-nitrophenylalanine (1) is subjected to amide formation to yield compound (2) through the formation of the unsymmetrical isobutyl carbonic anhydride and displacement with a primary amine. Numerous secondary amines and alcohols under similar conditions may also be used under similar conditions. Alternatively, methods involving the use of condensation reagents for the formation of amides may also be employed (See Bodansky, M; Activation and Coupling. In Principles of Peptide Synthesis, 2^{nd} ed.; Springer Publishing: New York, 1993, pp 9-61). Additionally, numerous amine components could be introduced at this step to add variability to the analog synthesis.

Lastly, intermediate (2) is then carried on directly to a final aryl sulfamic acid compound (3) through a two-step process, which involves aryl nitro group reduction (methods for which are numerous, see, e.g., Hudlicky, M; Reduction of Nitro, Nitroso, Diazo, and Azido Derivatives of Hydrocarbons and Basic Heterocycles, In Reductions in Organic Chemistry, 2^{nd} ed.; ACS Monograph 188; American Chemical Society: Washington, D.C., 1996) followed by sulfamic acid formation. Formation of the final sulfamic acid compounds is carried out by dissolution of the reduction product in anhydrous pyridine (ca. 2-3 mL per 0.5 mmol) and addition of solid sulfur trioxide pyridine complex (3 molar eqs.). Upon addition of the sulfur trioxide pyridine complex the reaction mixture is stirred for about 5 min then the reaction is quenched with diluted ammonium hydroxide solution (ca. 7% aqueous). Evaporation of all volatiles provides the crude material, which is generally purified by RP-HPLC to provide the target compounds in typical yields of 30-65% (2-steps). Additionally, alternative complexes of sulfur trioxide could be employed (i.e. sulfur trioxide dioxane, etc.), with non-limiting examples discussed in Gilbert (Chem. Rev. (1962) 62, 549-89). The reduced nitro compound can also be functionalized as the sulfamic acid by the action of chlorosulfonic acid in the presence of an appropriate base (see, e.g., Sureau, R. F. M, et. al; Preparation of Sulphamic Acids., U.S. Pat. No. 2,789,132) and also by the action of O-Trimethylsilyl chlorosulfonic acid and an appropriate base.

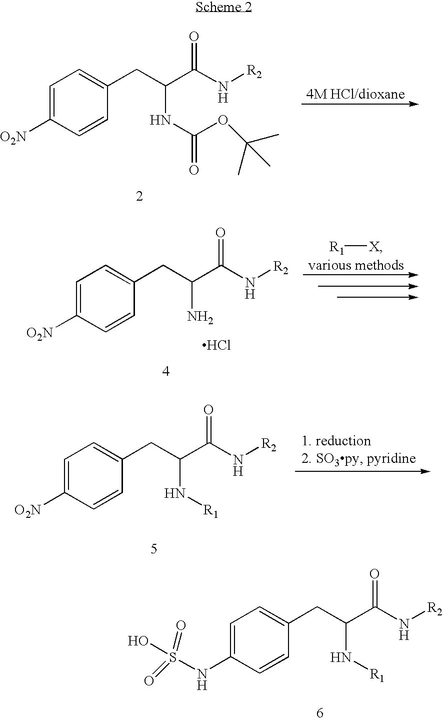

Alternatively, as shown in Scheme 2, intermediate 2 may be used as a starting point for other analogs after removal of the BOC group. Removal of the Boc protecting group is carried out under acidic conditions (see, e.g., Greene and Wuts, pp 518-525) such as using 4 M hydrogen chloride in 1,4-dioxane to provide the hydrochloride (4) in high yield and purity.

Intermediate (4) is the substrate for a number of subsequent functionalization reactions whereby numerous electrophilic reagents (R₂—X) are introduced under the appropriate conditions. Non-limiting examples of these types of reactions include urea formation by the addition of isocyanates, thio-urea formation by the addition of isothiocyanates, amide formation by the reaction with acid chlorides, amide formation through condensation with carboxylic acids employing an appropriate condensation reagent (See Bodansky, M; vide supra), carbamate formation by the reaction of chloroformates (see- Greene and Wuts, Protective Groups in Organic Synthesis, 3^{rd} ed.; Wiley & Sons: New York, 1999), amino compounds through reductive alkylation with suitable carbonyl compounds, amine formation through alkylation with alkyl halides, guanidine formation through various methods (see-Burgess, K; Chen, J.; Solid-Phase Synthesis of Guanidines. In Solid Phase Organic Synthesis; Kevin Burgess, Ed.; John Wiley & Sons: New York, 2000; pp 1-23: and references therein) and sulfonamide formation through reaction with sulfonyl chlorides. It is also possible to employ two of the above reaction types, such as reductive alkylation followed by acylation. The above-mentioned functionalization reactions are not to be considered exhaustive but merely illustrative of the types of routine chemistry, which can be carried out by those skilled in the art of organic synthesis to provide compounds of general structure (5).

Lastly, as in Scheme 1, intermediate 5 is then carried on directly to yield the final aryl sulfamic acid compounds of formula (6) through a two-step process, which involves aryl nitro group reduction followed by sulfamic acid formation; and optionally functionalizing thereafter.

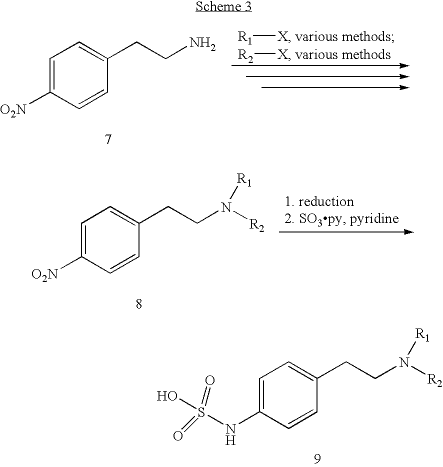

Compound 7 is a substrate for a number of functionalization reactions whereby electrophilic reagents are (R¹—X, optionally R₂—X) introduced under the appropriate conditions to provide intermediates of general structure 8. Lastly, as in Scheme 1, intermediate 8 is then carried on directly to yield the final aryl sulfamic acid compounds of formula (9) through a two-step process, which involves aryl nitro group reduction followed by sulfamic acid formation; and optionally functionalizing thereafter.

A variety of compounds can be generated in a similar fashion, using the guidance of the schemes above.

These steps may be varied to increase yield of desired product. The skilled artisan will recognize the judicious choice of reactants, solvents, and temperatures is an important component in any successful synthesis. Determination of optimal conditions, etc. is routine. Thus the skilled artisan can make a variety of compounds using the guidance of the schemes above.

It is recognized that the skilled artisan in the art of organic chemistry can readily carry out standard manipulations of organic compounds without further direction; that is, it is well within the scope and practice of the skilled artisan to carry out such manipulations. These include, but are not limited to, reduction of carbonyl compounds to their corresponding alcohols, oxidations of hydroxyls and the like, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (2 Volumes) and other art that the skilled artisan is aware of.

The skilled artisan will also readily appreciate that certain reactions are best carried out when another potentially reactive functionality on the molecule is masked or protected, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene, Protecting Groups in Organic Synthesis. Of course, amino acids used as starting materials with reactive side chains are preferably blocked to prevent undesired side reactions. The compounds of the invention may have one or more chiral centers. As a result, one may selectively prepare one optical isomer, including diastereomer and enantiomer, over another, for example by chiral starting materials, catalysts or solvents, or may prepare both stereoisomers or both optical isomers, including diastereomers and enantiomers at once (a racemic mixture). Since the compounds of the invention may exist as racemic mixtures, mixtures of optical isomers, including diastereomers and enantiomers, or stereoisomers may be separated using known methods, such as chiral salts, chiral chromatography and the like.

In addition, it is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

IV. Methods of Treating PTPase Mediated Disorders.

The above-identified compounds of the present invention may be used in a method for the treatment of a PTPase mediated disorder. As used herein, a “PTPase mediated disorder” is one that involves unwanted or elevated PTPase activity in the biological manifestation of the disease, disorder, and/or condition; in the biological cascade leading to the disorder; or as a symptom of the disorder. This “involvement” of PTPase in a PTPase mediated disorder includes, but is not limited to, the following: (1) The unwanted or elevated PTPase activity as a “cause” of the disorder or biological manifestation, whether the PTPase is elevated genetically, by infection, by autoimmunity, trauma, biomechanical causes, lifestyle, or by some other causes. (2) The unwanted or elevated PTPase activity is part of the observable manifestation of the disease or disorder. That is, the disease or disorder is measurable in terms of the increased PTPase activity. From a clinical standpoint, unwanted or elevated PTPase activity indicate the disease, however, PTPase activity need not be the “hallmark” of the disease or disorder. (3) The unwanted or elevated PTPase activity is part of the biochemical or cellular cascade that results in the disease or disorder. In this respect, inhibition of PTPase interrupts the cascade, and thus controls the disease. Non-limiting examples of PTPase mediated disorders that may be treated by the present invention include insulin receptor tyrosine mediated disorder, bone disorder, and vascular disorder.

As used herein, “PTPase” means enzymes with the capacity to dephosphorylate pTyr-containing proteins or glycoproteins or motifs generally. Non-limiting examples of PTPases include: intracellular PTPases (e.g., PTP1B, TC-PTP, PTP1C, PTPID, PTPD1, PTPD2); receptor-type PTPases (e.g., PTPα, PTPε, PTPβ, PTPγ, CD45, PTPκ, PTPμ); dual specificity phosphatases (VH1, VHR, cdc25), LMW-PTPases; and acid phosphatases.

All known intracellular type PTPases contain a single conserved catalytic phosphatase domain consisting of 220-240 amino residues. Non-limiting examples of intracellular type PTPases include: PTPI B (Tonks et al., J. Biol. Chem. 263: 6722-6730 (1988)); PTP1 (Charbonneau et al., Proc. Natl. Acad. Sci. USA 86: 5252-5256 (1989); Chemoff et al., Proc. Natl. Acad. Sci. USA 87: 2735-2789 (1989)); T-cell PTPase (Cool et al. Proc. Natl. Acad. Sci. USA 86: 5257-5261 (1989)); rat brain PTPase (Guan et al., Proc. Natl. Acad. Sci. USA 87: 1501-1502(1990)); neuronal phosphatase STEP (Lombroso et al., Proc. Natl. Acad. Sci. USA 88: 7242-7246 (1991)); ezrin-domain containing PTPases; PTPMEGI (Gu et al., Proc. Natl. Acad. Sci. USA 88: 5867-57871 (1991)), PTPH I Yang and Tonks, Proc. Natl. Acad. Sci. USA 88: 5949-5953 (1991), PTPD1 and PTPD2 (Moller et al., Proc. Natl. Acad. Sci. USA 91: 7477-7481 (1994)); FAP-1/BAS (Sato et al., Science 268: 411-415 (1995); Banville et al., J. Biol. Chem. 269: 22320-22327 (1994); Maekawa et al., FEBS Letters 337: 200-206 (1994)); and SH2 domain containing PTPases: PTP1C/SH-PTP1 (Plutzky et al., Proc. Natl. Acad. Sci. USA 89: 1123-1127 (1992); Shen et al., Nature Lond. 352: 736-739 (1991)) and PTP1D/Syp/SH-PTP2 (Vogel et al., Science 259: 1611-1614 (1993); Feng et al., Science 259: 1607-1611 (1993); Bastein et al., Biochem, Biophys. Res. Comm. 196: 124-133 (1993)).

Most receptor-type PTPases consist of a) a putative ligand-binding extracellular domain, b) a transmembrane segment, and c) an intracellular catalytic region. Non-limiting examples include CD45/LCA (Ralph, S. J., EMBO J. 6: 1251-1257 (1987)); LAR (Streuli et al., J. Exp. Med. 168:1523-1530 (1988); Charbonneau et al., Proc. Natl. Acad. Sci. USA 86: 5252-5256 (1989)); CD45 (Trowbridge and Thomas, Ann. Rev. Immunol. 12: 85-116 (1994)); PTPα Krueger et al., EMBO J. 9: 3241-3252 (1990)); PTPβ (Krueger supra); PTPδ (Krueger supra); PTPε (Krueger supra); PTP ζ (Krueger supra). Other examples of receptor type PTPases include PTPγ (Bamea et al., Mol. Cell. Biol. 13: 1497-1506 (1995); PTPμ (Gebbink et al., FEBS Letters 290: 123-130 (1991); PTPκ (Jiang et al., Mol. Cell. Biol. 13: 2942-2951 (1993); SAP-1 (Matozaki et al., J. Biol. Chem. 269: 2075-2081(1994)); and PTP-U2/GLEPP1 (Seimiya et al., Oncogene 10: 1731-1738 (1995); (Thomas et al., J. Biol. Chem. 269: 19953-19962 (1994)). Novel PTPases are continuously identified, and it is anticipated that more than 500 different species will be found in the human genome, i.e., close to the predicted size of the protein tyrosine kinase superfamily (Hanks and Hunter, FASEB J. 9: 576-596 (1995)).

Dual specificity protein tyrosine phosphatases (dsPTPases) define a subclass within the PTPases family that can hydrolyze phosphate from phosphotyrosine as well as from phosphoserine/threonine. dsPTPases contain the signature sequence of PTPases: His-Cys-Xxx-Xxx-Gly-Xxx-Xxx-Arg. At least three dsPTPases have been shown to dephosphorylate and inactivate extracellular signal-regulated kinase (ERKs)/mitogen-activated protein kinase (MAPK): MAPK phosphatase (CL100, 3CH134) (Charles et al., Proc. Natl. Acad. Sci. USA 90: 5292-5296 (1993)); PAC-1 (Ward et al., Nature 367: 651-654 (1994)); rVH6 (Mourey et al., J. Biol. Chem. 271: 3795-3802 (1996)). Transcripton of dsPTPases are induced by different stimuli, e.g., oxidative stress or heat shock (Ishibashi et al., J. Biol. Chem. 269: 29897-29902 (1994); Keyse and Emslie, Nature 359: 644-647 (1992)). Further, they may be involved in regulation of the cell cycle: cdc25 (Millar and Russell, Cell 68: 407-410 (1992)); KAP (Hannon et al. Proc. Natl. Acad. Sci. USA 91: 1731-1735 (1994); review by Walton and Dixon, Annu. Rev. Biochem. 62:101-120 (1993)).

Low molecular weight phosphotyrosine-protein phosphatase (LMW-PTPase) shows little sequence identity to the intracellular PTPases described above. However, this enzyme belongs to the PTPase family due to at least possessing the PTPase active site motif (Cirri et al., Eur. J. Biochem. 214: 647.657 (1993). For further rationales, see Chiarugi et al., FEBS Lett. 310: 9-12 (1992) and Su et al., Nature 370: 575-578 (1994).

To determine and assess the PTPase inhibition activity testing of the subject compounds is carried using various assays known to those skilled in the art. For example, a DiFMUP Phosphatase Assay is described. DiFMUP (“6,8-difluoro-4-methylumbelliferyl phosphate”) (Molecular Probes) (10 MM) is incubated for 15 minutes with nM concentrations of phosphatase in buffer containing 50 mM Tris (pH 7), 150 mM NaCl, 5 mM DTT, 1 mM EDTA, 0.01% BSA. The resulting phosphatase product is measured at 355/460 nm (ex/em) using a Victor V plate reader (Wallac). Inhibitors (0.002-40 MM) are pre-incubated with phosphatase for 10 minutes prior to addition of DIFMUP substrate. IC₅₀ curves are generated using Excel-Fit

C. Methods of Treatment

The compounds of the present invention may be useful in a method of treating a PTPase mediated disorder in a subject in need of such treatment comprising administering of a compound of the present invention.

The term “treatment” is used herein to mean that, at a minimum, administration of a compound of the present invention mitigates a disease associated with a PTPase mediated disorder in a subject, preferably in a mammalian subject, more preferably in humans. Thus, the term “treatment” includes: preventing an PTPase mediated disorder in a subject, particularly when the subject is predisposed to acquiring the disease, but has not yet been diagnosed with the disease; inhibiting the PTPase mediated disorder; and/or alleviating or reversing the PTPase mediated disorder. Insofar as the methods of the present invention are directed to preventing PTPase mediated disorder, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to PTPase mediated disorder, such that administration of the compounds of the present invention may occur prior to onset of PTPase mediated disorder. The term does not imply that the disease state be completely avoided. The population that is at risk of a PTPase mediated disorder, for example as diabetes type I, are those who have a genetic predisposition to diabetes as indicated by family history of the disease. Other risk factors include obesity or diet.

Different embodiments of PTPase mediated disorders of the present invention herein follow.

1. Insulin Receptor Mediated Disorder.

In one aspect of the invention, the PTPase mediated disorder is an insulin receptor tyrosine kinase mediated disorder. As used herein, “insulin receptor tyrosine mediated disorder” is a disease or disorder that involves defects in insulin receptor tyrosine signaling thereby resulting in the biological manifestation of the disorder; in the biological cascade leading to the disorder; or as a symptom of the disorder. In one embodiment, the insulin receptor tyrosine kinase mediated disorder is selected from the group consisting of type I diabetes, type II diabetes, impaired glucose tolerance, insulin resistance and obesity. In another embodiment, the disorder is type II diabetes.

In order to determine and assess the pharmacological activity against an insulin receptor tyrosine kinase mediated disorder, testing of the subject compounds in animals is carried using various assays known to those skilled in the art. For example, the activity of the subject compounds against diabetes can be measured using an assay designed to measure blood sugar levels in mice with diabetes experimentally induced by alloxan.

2. Bone Disorders.

In one aspect of the invention, the PTPase mediated disorder is a bone disorder. As used herein, “bone disorder” is a disease or disorder that involves defects in osteoblast proliferation thereby resulting in the biological manifestation of the disorder; in the biological cascade leading to the disorder; or as a symptom of the disorder. In one embodiment, the bone disorder is selected from the group consisting of osteoporosis and Paget's disease.

In order to determine and assess the pharmacological activity against a bone disorder, testing of the subject compounds in animals is carried out using various assays known to those skilled in the art. For example, the activity of the subject compounds against a bone disorder can be conveniently demonstrated using an assay designed to test the ability of the subject compounds to increase bone volume, mass, or density. An example of such an assay is the ovariectomized rat assay. In the overiectomized rat assay, six-month old rats are ovariecotmized, aged 2 months, and the dosed once a day subcutaneously with a test compound. Upon completion of the study, bone mass and/or density can be measured by dual energy X-ray absorptometry (DXA) or peripheral quantitative computed tomography (pQCT), or micro computed tomography (mCT). Alternatively, static and dynamic histomorphometry can be used to measure the increase in bone volume or formation.

3. Angiogenesis-Mediated Disorders

In one aspect of the invention, the PTPase mediated disorder is an angiogenesis mediated disorder. As used herein, “angiogenesis” means the formation of new blood vessels from pre-existing vasculature. As used herein, “angiogenesis mediated disorders” include: (1) those disorders, diseases and/or unwanted conditions which are characterized by unwanted or elevated angiogenesis referred to herein collectively as “angiogenesis elevated disorders;” or (2) those disorders, diseases and/or unwanted conditions which are characterized by wanted or reduced angiogenesis referred to herein collectively as “angiogenesis reduced disorders.”

a. Angiogenesis Elevated Disorder

As used herein, an “angiogenesis elevated disorder” is one that involves unwanted or elevated angiogenesis in the biological manifestation of the disease, disorder, and/or condition; in the biological cascade leading to the disorder; or as a symptom of the disorder. This “involvement” of angiogenesis in an angiogenesis elevated disorder includes, but is not limited to, the following: (1) The unwanted or elevated angiogenesis as a “cause” of the disorder or biological manifestation, whether the level of angiogenesis is elevated genetically, by infection, by autoimmunity, trauma, biomechanical causes, lifestyle, or by some other causes. (2) The angiogenesis as part of the observable manifestation of the disease or disorder. That is, the disease or disorder is measurable in terms of the increased angiogenesis. From a clinical standpoint, unwanted or elevated angiogenesis indicate the disease, however, angiogenesis need not be the “hallmark” of the disease or disorder. (3) The unwanted or elevated angiogenesis is part of the biochemical or cellular cascade that results to the disease or disorder. In this respect, inhibition of angiogenesis interrupts the cascade, and thus controls the disease. Non-limiting examples of angiogenesis reduced disorders that may be treated by the present invention are herein described below.

The compounds of the present invention may be used to treat diseases associated with retinal/choroidal neovascularization that include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and postlaser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

Compounds of the present invention can treat diseases associated with chronic inflammation. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis by the compositions and methods of the present invention would prevent the formation of the granulomas and alleviate the disease.

Compounds may be used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the compounds of the present invention inhibits the formation of the sprouts and prevents the formation of granulomas. Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The compounds of the present invention may be capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

Compounds of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease that may be treated according to the present invention, is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that can be treated according to the present invention are hemangiomas, Osler-Weber-Rendu disease, or hereditary hemorrhagic telangiectasia, solid or blood borne tumors and acquired immune deficiency syndrome.

b. Angiogenesis Reduced Disorder

As used herein, an “angiogenesis reduced disorder” is one that involves wanted or stimulated angiogenesis to treat a disease, disorder, and/or condition. The disorder is one characterized by tissue that is suffering from or be at risk of suffering from ischemic damage, infection, and/or poor healing, which results when the tissue is deprived of an adequate supply of oxygenated blood due to inadequate circulation. As used herein, “tissue” is used in the broadest sense, to include, but not limited to, the following: cardiac tissue, such as myocardium and cardiac ventricles; erectile tissue; skeletal muscle; neurological tissue, such as from the cerebellum; internal organs, such as the brain, heart, pancreas, liver, spleen, and lung; or generalized area of the body such as entire limbs, a foot, or distal appendages such as fingers or toes.

i. Methods of Vascularizing Ischemic Tissue

In one aspect in the method for the treatment of an angiogenesis reduced disorders, a compound of the invention may be used in a method of vascularizing ischemic tissue. As used herein, “ischemic tissue,” means tissue that is deprived of adequate blood flow. Examples of ischemic tissue include, but are not limited to, tissue that lack adequate blood supply resulting from mycocardial and cerebral infarctions, mesenteric or limb ischemia, or the result of a vascular occlusion or stenosis. In one example, the interruption of the supply of oxygenated blood may be caused by a vascular occlusion. Such vascular occlusion can be caused by arteriosclerosis, trauma, surgical procedures, disease, and/or other indications. There are many ways to determine if a tissue is at risk of suffering ischemic damage from undesirable vascular occlusion. Such methods are well known to physicians who treat such conditions. For example, in myocardial disease these methods include a variety of imaging techniques (e.g., radiotracer methodologies, x-ray, and MRI) and physiological tests. Therefore, induction of angiogenesis in tissue affected by or at risk of being affected by a vascular occlusion is an effective means of preventing and/or attenuating ischemia in such tissue. Thus, the treatment of skeletal muscle and myocardial ischemia, stroke, coronary artery disease, peripheral vascular disease, coronary artery disease are fully contemplated.

Any person skilled in the art of using standard techniques can measure the vascularization of tissue. Non-limiting examples of measuring vascularization in a subject include: SPECT (single photon emission computed tomography); PET (positron emission tomography); MRI (magnetic resonance imaging); and combination thereof, by measuring blood flow to tissue before and after treatment. Angiography can be used as an assessment of macroscopic vascularity. Histologic evaluation can be used to quantify vascularity at the small vessel level. These and other techniques are discussed in Simons, et al., “Clinical trials in coronary angiogenesis,” Circulation, 102, 73-86 (2000).

ii. Methods of Repairing Tissue

In one aspect in the method for the treatment of an angiogenesis reduced disorders, a compound of the present invention may be used in a method of repairing tissue. As used herein, “repairing tissue” means promoting tissue repair, regeneration, growth, and/or maintenance including, but not limited to, wound repair or tissue engineering. One skilled in the art readily appreciates that new blood vessel formation is required for tissue repair. In turn, tissue may be damaged by, including, but not limited to, traumatic injuries or conditions including arthritis, osteoporosis and other skeletal disorders, and burns. Tissue may also be damaged by results from injuries due to surgical procedures, irradiation, laceration, toxic chemicals, viral infection bacterial infection or burns. Tissue in need of repair also includes non-healing wounds. Non-limiting examples of non-healing wounds include: non-healing skin ulcers resulting from diabetic pathology; or fractures that do not heal readily.

Compounds of the invention may also be used in a method to aid in tissue repair in the context of guided tissue regeneration (GTR) procedures. Such procedures are currently used by those skilled in the medical arts to accelerate wound healing following invasive surgical procedures.

Compounds of the invention may be used in a method of promoting tissue repair characterized by enhanced tissue growth during the process of tissue engineering. As used herein, “tissue engineering” is defined as the creation, design, and fabrication of biological prosthetic devices, in combination with synthetic or natural materials, for the augmentation or replacement of body tissues and organs. Thus, the present method can be used to augment the design and growth of human tissues outside the body for later implantation in the repair or replacement of diseased tissues. For example, compounds of the invention may be useful in promoting the growth of skin graft replacements that are used as a therapy in the treatment of burns.

In another aspect of tissue engineering, compounds of the present invention may be included in cell-containing or cell-free devices that induce the regeneration of functional human tissues when implanted at a site that requires regeneration. As previously discussed, biomaterial-guided tissue regeneration can be used to promote bone regrowth in, for example, periodontal disease. Thus, an AMP may be used to promote the growth of reconstituted tissues assembled into three-dimensional configurations at the site of a wound or other tissue in need of such repair.

In another aspect of tissue engineering, compounds of the invention can be included in external or internal devices containing human tissues designed to replace the function of diseased internal tissues. This approach involves isolating cells from the body, placing them on or within structural matrices, and implanting the new system inside the body or using the system outside the body. The method of the invention can be included in such matrices to promote the growth of tissues contained in the matrices. For example, a compound can be included in a cell-lined vascular graft to promote the growth of the cells contained in the graft. It is envisioned that the method of the invention can be used to augment tissue repair, regeneration and engineering in products such as cartilage and bone, central nervous system tissues, muscle, liver, and pancreatic islet (insulin-producing) cells.

4. Vascular Tone Mediated Disorders

In one aspect of the invention, the PTPase mediated disorder is a vascular tone mediated disorder. As used herein, “vascular tone mediated disorder” is a disease or disorder that involves defects in endothelial PTK signaling thereby resulting in the biological manifestation of the disorder; in the biological cascade leading to the disorder; or as a symptom of the disorder. In one embodiment, the vascular tone mediated disorder is selected from the group consisting of primary essential hypertension, secondary hypertension, pulmonary hypertension and portal hypertension.

5. Vascular Permeability Mediated Disorders

In one aspect of the invention, the PTPase mediated disorder is a vascular permeability mediated disorder. As used herein, “vascular tone mediated disorder” is a disease or disorder that involves defects in VEGF induced vascular permeability thereby resulting in the biological manifestation of the disorder; in the biological cascade leading to the disorder; or as a symptom of the disorder. In one embodiment, the vascular permeability mediated disorder is selected from the group consisting of stroke, septic shock, burns, respiratory distress syndrome and congestive heart failure.

6. VEGF Mediated Disorders

In one aspect of the invention, the PTPase mediated disorder is a VEGF mediated disorder. As used herein, “VEGF mediated disorder” is a disease or disorder that involves defects in VEGF signaling thereby resulting in the biological manifestation of the disorder; in the biological cascade leading to the disorder; or as a symptom of the disorder. In one embodiment, the VEGF mediated disorder is selected from the group consisting of heart failure, myocardial infarction (MI), diabetic and ischemic neuropathy, osteoporosis, bone fracture healing, wound healing and hair loss.

A suitable MI cardiac pharmacological model is described in Mukherjee, R. et al., J. Cardiac Failure;7 Suppl 2:7 (2001). Briefly, pigs are prepared for the induction of myocardial infarction by implantation of an occlusion device on the circumflex coronary artery, and radiopaque markers are placed in the region destined to be infarcted to measure infarct expansion (see below). Measurements of left ventricular (hereinafter “LV”) volumes and distances between marker beads are made prior to and at various times after the induction of MI induced by activating the occlusion device.

The effects of compounds of the present invention effective in the treatment of MI may be studied in a pig model of MI induced by ligation of the circumflex coronary artery. Animals are assigned to one of the following treatment groups: (1) 1 or 10 mg/kg three times a day of a compound of Formula (I) by oral administration starting 3 days prior to myocardial infarction; (2) 10 mg/kg three times a day of said compound by oral administration starting 3 days after MI; (3) MI with no active treatment; or (4) no myocardial infarction or drug treatment. At 10 days post-MI, LV end-diastolic volume (hereinafter “LVEDV”) is measured by ventriculography. LVEDV is increased in all MI groups. An attenuated increase in LVEDV by a compound of Formula (I) indicates that the compound may be effective in the prevention or treatment of progressive ventricular dilation, and thus the subsequent development of CHF.

V. Compositions

The subject compounds can be administered as a composition that comprise: (a) a safe and effective amount of a compound of the invention; and (b) a pharmaceutically-acceptable carrier. The subject compositions may be useful for the treatment of PTPase mediated disorders.

A “safe and effective amount” of a subject compound is an amount that is effective, to treat a PTPase mediated disorder, without undue adverse side effects (such as toxicity, irritation, or allergic response), commensurate with a reasonable benefit/risk ratio when used in the manner of this invention. The specific “safe and effective amount” will vary with such factors as the particular condition being treated, the physical condition of the patient, the duration of treatment, the nature of concurrent therapy (if any), the specific dosage form to be used, the excipient employed, the solubility of the subject compound therein, and the dosage regimen desired for the composition. The term “pharmaceutically-acceptable carrier”, as used herein, means one or more compatible solid or liquid filler diluents or encapsulating substances which are suitable for administration to an animal, preferably a mammal, more preferably a human. The term “compatible”, as used herein, means that the components of the composition are capable of being commingled with the subject compound, and with each other, in a manner such that there is no interaction that would substantially reduce the pharmaceutical efficacy of the composition under ordinary use situations. Pharmaceutically-acceptable carriers must, of course, be of sufficiently high purity and sufficiently low toxicity to render them suitable for administration to the subject, preferably a mammal, more preferably a human being treated.

Some examples of substances which can serve as pharmaceutically-acceptable carriers or components thereof are: sugars, such as lactose, glucose and sucrose; starches; cellulose, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of a subject compound that is suitable for administration to a subject according to good medical practice. These compositions preferably contain from about 5 mg (milligrams) to about 1000 mg, more preferably from about 10 mg to about 500 mg, more preferably from about 10 mg to about 300 mg, of a subject compound.

The compositions of this invention may be in any of a variety of forms, suitable, for example, for oral, rectal, topical, nasal, ocular or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the inhibitory activity of the subject compound. The amount of carrier employed in conjunction with the subject compound is sufficient to provide a practical quantity of material for administration per unit dose of the subject compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the Formula (I) compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, and containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit®coatings, waxes and shellac.

Other compositions useful for attaining systemic delivery of the subject compounds include sublingual, buccal, suppository, and nasal dosage forms.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions preferably comprise a safe and effective amount, usually at least about 0.1%, and preferably from about 1% to about 5%, of the Formula (I) compound. Suitable carriers for topical administration preferably remain in place on the skin as a continuous film, and resist being removed by perspiration or immersion in water. Generally, the carrier is organic in nature and capable of having dispersed or dissolved therein the Formula (I) compound. The carrier may include pharmaceutically-acceptable emollients, emulsifiers, thickening agents, solvents and the like.

The specific dosage of subject compound or composition to be administered, as well as the duration of treatment, are mutually dependent. The dosage and treatment regimen will also depend upon such factors as the specific subject compounds used, the specific PTPase mediated disorder, the ability of the subject compound to reach minimum inhibitory concentrations at the site of the disorder, the nature and extent of other disorder (if any), the personal attributes of the subject (such as weight), compliance with the treatment regimen, the age and health status of the patient, and the presence and severity of any side effects of the treatment.

Typically, for a human adult (weighing approximately 70 kilograms), from about 75 mg, more preferably from about 200 mg, most preferably from about 500 mg to about 30,000 mg, more preferably to about 10,000 mg, most preferably to about 3,500 mg, of a subject compound is administered per day. Treatment regimens preferably extend from about 1, preferably from about 3 to about 56 days, preferably to about 20 days, in duration. Prophylactic regimens (such as prevention of osteoporosis) may extend 6 months, or longer, according to good medical practice.

### VI. EXAMPLES

The R groups used to illustrate the compound examples of this section VI may not correlate to the respective R group used to describe the various moieties of Formula (I).

### Examples 1-36

The following chemical formula along with Table 1 shows the structure of compounds according to the description in Examples 1-36 described below.

TABLE 1

| | |
|---|---|
| | Formula (II) |
| 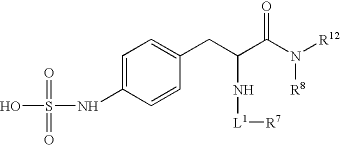 | |

| EXAMPLE | * | L¹ | R⁷ | R⁸ | R¹² |
|---|---|---|---|---|---|
| 1 | R | —CO₂— | 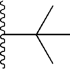 | H | —CH₃ |
| 2 | R | —CO₂— | 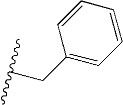 | H | —CH₃ |
| 3 | S | —CO₂— | 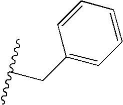 | H | —CH₃ |
| 4 | S | —CO₂— | 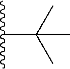 | H | —CH₃ |
| 5 | R | —CO₂— | 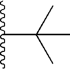 | H | 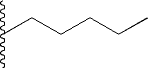 |
| 6 | R | —CO₂— | 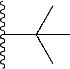 | H | 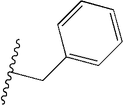 |
| 7 | S | —CO₂— | 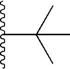 | H | 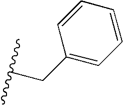 |
| 8 | R | —CO₂— | 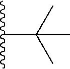 | H | 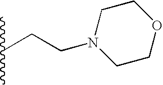 |
| 9 | S | —CO₂— | 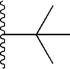 | H | 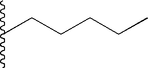 |
| 10 | S | —CO— | 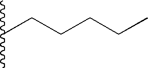 | H | —CH₃ |
| 11 | S | —SO₂— | 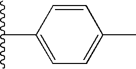 | H | —CH₃ |
| 12 | R | —CO— | 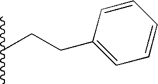 | H | —CH₃ |
| 13 | S | —CO— | 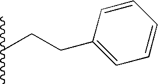 | H | —CH₃ |
| 14 | S | —CO₂— | 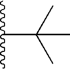 | H | 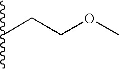 |
| 15 | S | —CO₂— | 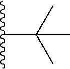 | H | 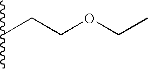 |
| 16 | S | —CO₂— | 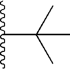 | H | 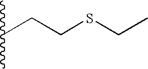 |
| 17 | S | —CO₂— | 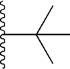 | H | 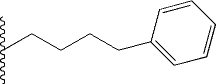 |
| 18 | S | —CO₂— | 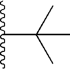 | H | 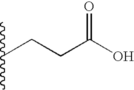 |
| 19 | S | —CONH— | 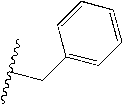 | H | —CH₃ |
| 20 | S | —CONH— | 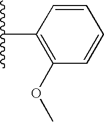 | H | —CH₃ |
| 21 | S | —SO₂— | 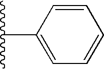 | H | —CH₃ |
| 22 | S | —SO₂— | 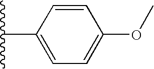 | H | —CH₃ |
| 23 | S | —SO₂— | 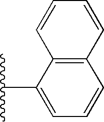 | H | —CH₃ |
| 24 | S | —CO₂— | 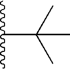 | —CH₃ | 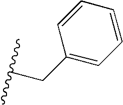 |
| 25 | S | —CO₂— | 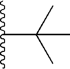 | H | 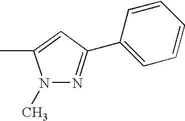 |
| 26 | S | —CO₂— | 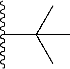 | H | 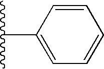 |
| 27 | S | —CO₂— | 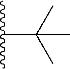 | 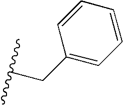 | 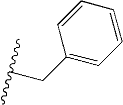 |
| 28 | S | —CO— | 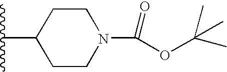 | H | —CH₃ |
| 29 | S | —CO— | 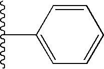 | H | —CH₃ |
| 30 | S | —CO₂— | 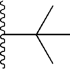 | —CH₃ | —CH₃ |
| 31 | S | —CO— | 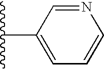 | H | —CH₃ |
| 32 | S | —CO— | 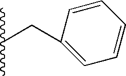 | H | —CH₃ |
| 33 | S | —CO— | 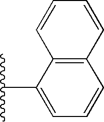 | H | —CH₃ |
| 34 | S | —CO— | 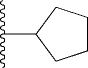 | H | —CH₃ |
| 35 | S | —CO— | 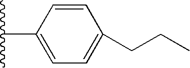 | H | 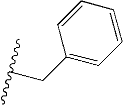 |
| 36 | S | —CO— | 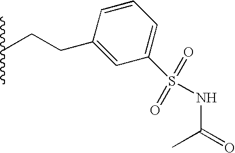 | H | —CH₃ |

### Example 1

(R)-[1-Methylcarbamoyl-2-(4-sulfoamino-phenyl)-ethyl]-carbamic acid tert-butyl ester:

Procedure A:

Boc-D-Phe(4-NO₂)—NMe: Boc-D-Phe(4-NO₂)—OH (4.0 g, 12.9 mmol) is dissolved in anhydrous tetrahydrofuran (20 mL) with 4-methylmorpholine (1.56 mL, 14.2 mmol). Isobutylchloroformate (1.84 mL, 14.2 mmol) is added dropwise at 0° C. and the mixture is stirred for 1 hr. at 0° C. Methylamine (12.9 mL, 2.0 M in tetrahydrofuran) is added dropwise at 0° C. and the mixture is stirred for 18 hr. at room temperature. The mixture is then recrystallized from 1:1 DCM:methanol to give a white solid.

Boc-D-Phe (4-NH₂)—NMe: Boc-D-Phe(4-NO₂)—NMe is dissolved in methanol (10 mL). To this was added palladium on carbon (10% by weight, 100 mg). The reaction is placed under a hydrogen atmosphere until reaction is complete (tlc). The catalyst is removed by filtration and the filtrate is concentrated to provide the amine, which is used without purification.

(R)-[1-Methylcarbamoyl-2-(4-sulfoamino-phenyl)-ethyl]-carbamic acid tert-butyl ester: In a dry flask 0.160 g of the aniline compound is dissolved in 2 mL pyridine. To this solution is added 0.130 g of sulfurtrioxide-pyridine complex. The mixture is stirred 5 minutes then diluted with 25 mL of 7% ammonium hydroxide. The mixture is evaporated down to an off-white solid and purified to provide 0.091 g of product as its ammonium salt. ¹H(D₂O): 7.07-7.00 (q, 4H, J=10.0 Hz), 4.05 (t, 1H, J=7.3 Hz), 2.91-2.69 (m, 2H) 2.53 (s, 3H), 1.22 (s, 9H).

### Example 2

(R)-[1-Methylcarbamovl-2-(4-sulfoamino-phenyl)-ethyl]-carbamic acid benzyl ester:

Procedure B:

H-D-Phe(4-NO₂)—NMe: Boc-D-Phe(4-NO₂)—NMe (1.5 g, 4.64 mmol) is dissolved in HCl (10 mL, 4.0 M in 1,4-dioxane), and the resulting mixture is stirred at room temperature for 1 hr. Ether (60 mL) is added to the mixture and the resulting precipitate is collected by filtration to yield pure white product.

CBZ-D-Phe(4-NO₂)—NMe: H-D-Phe(4-NO₂)—NMe (410 mg, 1.84 mmol) is dissolved in anhydrous DCM (10 mL) and diisopropylethylamine (0.352 mL, 2.02 mmol). Benzyl chloroformate (0.263 mL, 1.84 mmol) is added dropwise at 0° C. The mixture is allowed to warm to room temperature and is stirred for 72 hr. The solution is partitioned between DCM and 1N HCl. The organic layer is washed with brine, dried over MgSO₄, filtered and evaporated to give crude white solid.

CBZ-D-Phe(4-NH₂)—NMe: CBZ-D-Phe(4-NO₂)—NMe (80 mg, 0.224 mmol) is dissolved in EtOAc: ethanol (1:1, 2 mL) and tin(II) chloride dihydrate (252 mg, 1.12 mmol) is added. The mixture is stirred at room temperature for 18 h. The reaction is partitioned between EtOAc (25 mL) and 1N NaOH (25 mL). The organic layer was washed twice more with 1N NaOH (25 mL). The combined organics were dried over MgSO4, filtered and evaporated to give pure yellow oil. (R)-[1-Methylcarbamoyl-2-(4-sulfoamino-phenyl)-ethyl]-carbamic acid benzyl ester: In a similar manner to Procedure A, 0.107 g of aniline compound is treated with 0.156 g of sulfurtrioxide-pyridine complex. Work up and purification yields 0.056 g of product as its ammonium salt. ¹H(D₂O) 7.26-7.20 (m, 3H), 7.11-6.96 (m, 6H), 4.904.78 (m, 2H), 4.08 (t, 1H, J=8.3 Hz), 2.84-2.66 (m, 2H) 2.50 (s, 3H).

### Example 3

(S)-[1-Methylcarbamoyl-2-(4-sulfoamino-phenyl)-ethyl]-carbamic acid benzyl ester:

(S)-[1-Methylcarbamoyl-2-(4-nitrophenyl)-ethyl]-carbamic acid benzyl ester: In a manner similar to Example 1, 5.00 gram of Boc-L-Phe(4-NO₂)—OH was treated with 1.79 grams of NMM, 2.20 grams of isobutyl chloroformate, and 16.1 mL of methylamine solution. This affords the product as white solid. In a manner similar to Example 1, 1.11 gram of Boc-L-Phe (4-NH₂)—NMe and 100 mg of palladium on carbon is reduced to give the desired product.

(S)-[1-Methylcarbamoyl-2-(4-sulfoamino-phenyl)-ethyl]-carbamic acid benzyl ester: In a similar manner to procedure A, 0.149 g of aniline compound is treated with 0.218 g of sulfurtrioxide-pyridine complex. Work up and purification yields 0.091 g of product as its ammonium salt. ¹H(D₂O): 7.20-7.14 (m, 3H), 7.05-6.94 (m, 6H), 4.844.72 (m, 2H), 4.06 (t, 1H, J=7.7 Hz), 2.80-2.62 (m, 2H) 2.47 (s, 3H)

### Example 4

(S)-[1-Methylcarbamoyl-2-(4-sulfoamino-phenyl)-ethyl]-carbamic acid tert-butyl ester:

(S)-[1-Methylcarbamoyl-2-(4-nitrophenyl)-ethyl]-carbamic acid tert-butyl ester: In a manner similar to procedure A, 0.500 g of nitro compound and 0.05 g of palladium on carbon is reduced to give the desired product.

(S)-[1-Methylcarbamoyl-2-(4-sulfoamino-phenyl)-ethyl]-carbamic acid tert-butyl ester: In a similar manner to procedure A, 0.420 g of aniline compound is treated with 0.684 g of sulfurtrioxide-pyridine complex. Work up and purification yields 0.106 g of product as its ammonium salt. ¹H(D₂O): 7.04 (s, 4H), 4.07-4.05 (m, 1H), 2.92-2.68 (m, 2H) 2.55 (s, 3H), 1.24 (s, 9H).

### Example 5

(R)-[1-Pentylcarbamoyl-2-(4-suffoamino-phenyl)-ethyl]-carbamic acid tert-butyl ester:

(R)-[1-Pentylcarbamoyl-2-(4-nitrophenyl)-ethyl]-carbamic acid tert-butyl ester: In a manner similar to example 1, 5.00 gram of Boc-D-Phe(4-NO₂)—OH is treated with 1.79 grams of NMM, 2.40 grams of isobutyl chloroformate, and 3.73 mL of amylamine. This affords the product as white solid.

(R)-[1-Pentylcarbamoyl-2-(4-sulfoamino-phenyl)-ethyl]-carbamic acid tert-butyl ester: In a similar manner to procedure A, 0.200 g of nitro compound and 100 mg of palladium on carbon is reduced to give the desired product (tlc). This crude aniline product is treated with 0.252 g of sulfurtrioxide-pyridine complex. Work up and purification yields 0.030 g of product as its ammonium salt. ¹H(D₂O) 7.03-6.85 (m, 4H), 3.95 (m, 1H), 2.93-2.68 (m, 4H) 1.14 (s, 9H), 1.10 (m, 6H), 0.61 (t, 3H, J=8.2 Hz).

### Example 6

(R)-[1-Benzylcarbamoyl-2-(4-sulfoamino-phenyl)-ethyl]-carbamic acid tert-butyl ester:

In a manner similar to example 1, 4.00 gram of Boc-D-Phe(4-NO₂)—OH is treated with 1.44 grams of NMM, 1.94 grams of isobutyl chloroformate, and 2.82 mL of benzylamine. This affords the product as yellow solid.

(R)-[1-Benzylcarbamoyl-2-(4-nitrophenyl)-ethyl]-carbamic acid tert-butyl ester: In a similar manner to procedure A, 0.300 g of nitro compound and 50 mg of palladium on carbon is reduced to give the desired product (tlc). This crude aniline compound is treated with 0.359 g of sulfurtrioxide-pyridine complex. Work up and purification yields 0.028 g of product as its ammonium salt. ¹H(D₂O): 7.26-7.23 (m, 3H), 7.05-6.97 (m, 6H), 4.29-4.08 (m, 3H), 3.04 (s, 2H) 1.26 (s, 9H).

### Example 7

(S)-[1-Benzylcarbamoyl-2-(4-sulfoamino-phenyl)-ethyl]-carbamic acid tert-butyl ester:

(S)-[1-Benzylcarbamoyl-2-(4-nitrophenyl)-ethyl]-carbamic acid tert-butyl ester: In a manner similar to example 1, 4.00 gram of Boc-L-Phe(4-NO₂)—OH was treated with 1.44 grams of NMM, 1.94 grams of isobutyl chloroformate, and 2.82 mL of benzylamine. This affords the product as yellow solid.

(S)-[1-Benzylcarbamoyl-2-(4-sulfoamino-phenyl)-ethyl]-carbamic acid tert-butyl ester: In a similar manner to procedure A, 0.300 g of nitro compound and 50 mg of palladium on carbon is reduced to give the desired product (tlc). This crude aniline compound is treated with 0.359 g of sulfurtrioxide-pyridine complex. Work up and purification yields 0.066 g of product as its ammonium salt. ¹H(D₂O) 7.10-6.98 (m, 3H), 6.87-6.71 (m, 6H), 4.07-3.81 (m, 3H), 2.79 (s, 2H) 1.04 (s, 9H).

### Example 8

(R)-[1-(2-Morpholin-4-yl-ethylcarbamoyl)-2-(4-sulfoamino-phenyl)-ethyl]-carbamic acid tert-butyl ester:

(R)-[1-(2-Morpholin-4-yl-ethylcarbamoyl)-2-(4nitro-phenyl)-ethyl]-carbamic acid tert-butyl ester: In a manner similar to example 1, 4.00 gram of Boc-D-Phe(4-NO₂)—OH is treated with 1.44 grams of NMM, 1.94 grams of isobutyl chloroformate, and 3.39 mL of 4-(2-aminomethyl)morpholine. This affords the product as white solid.

(R)-[1-(2-Morpholin-4-yl-ethylcarhamoyl)-2-(4-sulfoamino-phenyl)-ethyl]-carbamic acid tert-butyl ester: In a similar manner to procedure A, 0.300 g of nitro compound and 100 mg of palladium on carbon is reduced to give the desired product (tlc). This crude aniline compound is treated with 0.339 g of sulfurtrioxide-pyridine complex. Work up and purification yields 0.188 g of product as its ammonium salt. ¹H(D₂O): 7.07-7.00 (q, 4H, J=9.9 Hz), 4.07 (m, 1H), 3.69 (s, 4H) 3.28-3.24 (m, 2H), 2.76-2.57 (m, 8H), 1.25 (s, 9H).

### Example 9

(S)-[1-Pentylcarbamoyl-2-(4-sulfoamino-phenyl)-ethyl]-carbamic acid tert-butyl ester:

(S)-[1-Pentylcarbamoyl-2-(4-nitrophenyl)-ethyl]-carbamic acid tert-butyl ester: In a manner similar to example 1, 5.00 gram of Boc-L-Phe(4-NO₂)—OH is treated with 1.79 grams of NMM, 2.42 grams of isobutyl chloroformate, and 3.73 mL of amylamine. This affords the product as white solid.

(S)-[1-Pentylcarbamoyl-2-(4-sulfoamino-phenyl)-ethyl]-carbamic acid tert-butyl ester: In a similar manner to procedure A, 0.300 g of nitro compound and 100 mg of palladium on carbon is reduced to give the desired product (tlc). This crude aniline compound is treated with 0.378 g of sulfurtrioxide-pyridine complex. Work up and purification yields 0.78 g of product as its ammonium salt. ¹H(D₂O): 7.16-6.98 (m, 4H), 4.08 (m, 1H), 3.06-2.81 (m, 4H) 1.27 (s, 9H), 1.211.02 (m, 6H), 0.75 (t, 3H, J=7.1 Hz).

### Example 10

(S)-[4-(2-Hexanoylamino-2-methylcarbamoyl-ethyl)-phenyl]-sulfamic acid:

(S)-Hexanoic acid [1-methylcarbamoyl-2-(4-nitro-phenyl)-ethyl]-amide: H-Phe(4-NO₂)NMe (808 mg) is dissolved in 5 mL DCM with 0.86 mL triethylamine. Hexanoyl chloride (0.476 mL) is added dropwise at 0° C. and the mixture is stirred at room temperature for 1 hr. The mixture is then evaporated to dryness and recrystallized from DCM:methanol to give pure white product.

(S)-[4-(2-Hexanoylamino-2-methylcarbamoyl-ethyl)-phenyl]-sulfamic acid: In a similar manner to procedure A, 0.309 g of nitro compound and 100 mg of palladium on carbon is reduced to give the desired product (tlc). This crude aniline compound is treated with 0.460 g of sulfurtrioxide-pyridine complex. Work up and purification yield 0.046 g of product as its ammonium salt. ¹H(D₂O) 7.11-7.02 (q, 4H, J=10.9 Hz), 4.39 (t, 1H, J=10.0 Hz), 3.02-2.76 (m, 2H), 2.58 (s, 3H), 2.11 (t, 2H, J=8.0 Hz), 1.33 (t, 2H, J=8.2 Hz) 1.14 (t, 2H, J=8.0 Hz) 1.03-0.95 (m, 2H), 0.73 (t, 3H, J=8.1 Hz).

### Example 11

(S)-{4-[2-Methylcarbamoyl-2-(toluene-4-sulfonylamino)-ethyl]-phenyl}-sulfamic acid:

(S)—N-Methyl-3-(4-nitro-phenyl)-2-(toluene-4-sulfonylamino)-propionamide: H-Phe(4-NO₂)—NMe (0.200 g) is dissolved in anhydrous 5 mL DCM with 0.156 g triethylamine. p-Toluenesulfonyl chloride (0.161 g) is added dropwise and the mixture is stirred at room temperature for 18 hr. Hexanes (30 mL) is added to the mixture and the resulting solid is collected by filtration. The crude solid is purified by flash chromatography to yield 0.079 g of the desired product.

(S)-{4-[2-Methylcarbamoyl-2-(toluene-4-sulfonylamino)-ethyl]-phenyl}-sulfamic acid: In a similar manner to procedure A, 0.079 g of nitro compound and 50 mg of palladium on carbon is reduced to give the desired product (tlc). This crude aniline compound is treated with 0.100 g of sulfurtrioxide-pyridine complex. Work up and purification yields 0.020 g of product as its ammonium salt. ¹H(D₂O): 7.61-7.42 (q, 4H, J=9.2 Hz), 7.07 (s, 4H) 3.94 (t, 1H, J=6.5 Hz), 3.102.80 (m, 2H), 2.73 (s, 3H), 2.60 (s, 3H).

### Example 12

(R)-{4-[2-Methylcarbamoyl-2-(3-phenyl-propionylamino)-ethyl]-phenyl}-sulfamic acid:

(R)-N-Methyl-3-(4-nitro-phenyl)-2-(3-phenyl-propionylamino)-propionamide: In a manner similar to example 2, 0.200 g of H-D-Phe(4-NO₂)—NMe is treated with 0.164 g of triethylamine and 0.164 g of hydrocinnamoyl chloride. This affords 0.307 g of the desired product.

(R)-{4-[2-Methylcarbamoyl-2-(3-phenyl-propionylamino)-ethyl]-phenyl}-sulfamic acid: In a similar manner to procedure A, 0.307 g of nitro compound and 100 mg of palladium on carbon is reduced to give the desired product (tlc). This crude aniline compound is treated with 0.448 g of sulfurtrioxide-pyridine complex. Work up and purification yields 0.040 g of product as its ammonium salt. ¹H(D₂O): 7.24-7.20 (m, 3H), 7.07-6.91 (m, 6H) 4.27 (t, 1H, J=8.8 Hz), 2.87-2.67 (m, 4H), 2.52 (s, 3H), 2.45 (t, 2H, J=7.3 Hz).

### Example 13

(S)-{4-[2-Methylcarbamoyl-2-(3-phenyl-propionylamino)-ethyl]-phenyl}-sulfamic acid:

(S)-N-Methyl-3-(4-nitro-phenyl)-2-(3-phenyl-propionylamino)-propionamide: In a manner similar to example 2, 0.200 g of H-L-Phe(4-NO₂)—NMe is treated with 0.164 mg of triethylamine and 0.164 g of hydrocinnamoyl chloride. This affords 0.284 g of the desired product.

(S)-{4-[2-Methylcarbamoyl-2-(3-phenyl-propionylamino)-ethyl]-phenyl}-sulfamic acid: In a similar manner to procedure A, 0.284 g of nitro compound and 100 mg of palladium on carbon is reduced to give the desired product (tlc). This crude aniline compound is treated with 0.414 g of sulfurtrioxide-pyridine complex. Work up and purification yields 0.018 g of product as its ammonium salt. ¹H(D₂O) 7.24-7.17 (m, 3H), 7.07-6.95 (m, 6H) 4.26 (t, 1H, J=8.2 Hz), 2.87-2.67 (m, 4H), 2.51 (s, 3H), 2.45 (t, 2H, J=8.0 Hz).

### Example 14

(S)-[1-(2-Methoxy-ethylcarbamoyl)-2-(4-sulfoamino-phenyl)-ethyl]-carbamic acid tert-butyl ester:

(S)-[1-(2-Methoxy-ethylcarbamoyl)-2-(4-nitrophenyl)-ethyl]-carbamic acid tert-butyl ester: In a manner similar to example 1, 1.00 g of Boc-L-Phe(4-NO₂)—OH is treated with 0.359 g of NMM, 0.483 g of isobutyl chloroformate, and 0.541 mL of 2-methoxyethylamine. This affords 0.300 g of the desired product.

(S)-[1-(2-Methoxy-ethylcarbamoyl)-2-(4-sulfoamino-phenyl)-ethyl]-carbamic acid tert-butyl ester: In a similar manner to procedure A, 0.300 g of nitro compound and 100 mg of palladium on carbon is reduced to give the desired product (tlc). This crude aniline compound is treated with 0.390 g of sulfurtrioxide-pyridine complex. Work up and purification yields 0.181 g of product as its ammonium salt. ¹H(D₂O) 7.09-6.79 (q, 4H, J=8.9 Hz), 3.93 (m, 1H), 3.25-2.62 (m, 6H), 3.01 (s, 3H), 1.07 (s, 9H).

### Example 15

(S)-[1-(2-Ethoxy-ethylcarbamoyl)-2-(4-sulfoamino-phenyl)-ethyl]-carbamic acid tert-butyl ester:

(S)-[1-(2-Ethoxy-ethylcarbamoyl)-2-(4-nitrophenyl)-ethyl]-carbanmic acid tert-butyl ester: In a manner similar to example 1, 1.00 g of Boc-L-Phe(4-NO₂)—OH is treated with 359 mg of NMM, 483 mg of isobutyl chloroformate, and 0.675 mL of 2-ethoxyethylamine. This affords 0.312 g of the desired product.

(S)-[1-(2-Ethoxy-ethylcarbamoyl)-2-(4-sulfoamino-phenyl)-ethyl]-carbamic acid tert-butyl ester: In a similar manner to procedure A, 0.312 g of nitro compound and 100 mg of palladium on carbon is reduced to give the desired product (tlc). This crude aniline compound is treated with 0.391 g of sulfurtrioxide-pyridine complex. Work up and purification yields 0.157 g of product as its ammonium salt. ¹H(D₂O): 7.21-6.98 (q, 4H, J=10.2 Hz), 4.13 (t, 1H, J=7.0 Hz), 3.44-3.16 (m, 6H), 2.89-2.80 (m, 2H) 1.26 (s, 9H), 1.03 (t, 3H, J=7.9 Hz).

### Example 16

(S)-[1-(2-Ethylsulfanyl-ethylcarbamoyl)-2-(4-sulfoamino-phenyl)-ethyl]-carbamic acid tert-butyl ester:

(S)-[1-(2-Ethylsulfanyl-ethylcarbamoyl)-2-(4nitrophenyl)-ethyl]-carbamic acid tert-butyl ester: In a manner similar to example 1, 1.00 gram of Boc-Phe(4-NO₂)—OH is treated with 0.685 g of NMM, 0.483 g of isobutyl chloroformate, and 0.912 g of 2-(ethylthio)ethylarine hydrochloride. This affords 0.249 g of the desired product. (S)-[1-(2-Ethylsulfanyl-ethylcarbamoyl)-2-(4-sulfoamino-phenyl)-ethyl]-carbamic acid tert-butyl ester: In a similar manner to procedure A, 0.249 g of nitro compound and 50 mg of palladium on carbon is reduced to give the desired product (tlc). This crude aniline compound is treated with 0.299 g of sulfurtrioxide-pyridine complex. Work up and purification yields 0.044 g of product as its ammonium salt. ¹H(D₂O) 7.39-7.15 (q, 2H, J=9.4 Hz), 7.12-7.03 (q, 2H, J=7.3 Hz), 4.13 (t, 1H, J=8.8 Hz), 3.30-3.17 (m, 2H), 2.94-2.83 (m, 2H) 2.49-2.42 (m, 4H) 1.27 (s, 9H), 1.11 (t, 3H, J=8.2 Hz).

### Example 17

(S)-[1-(4-Phenyl-butylcarbamoyl)-2-(4-sulfoamino-phenyl)-ethyl]-carbamic acid tert-butyl ester:

(S)-[1-(4-Phenyl-butylcarbamoyl)-2-(4-nitrophenyl)-ethyl]-carbamic acid tert-butyl ester: In a manner similar to example 1, 1.00 g of Boc-L-Phe(4-NO₂)—OH is treated with 0.359 g of NMM, 0.483 g of isobutyl chloroformate, and 0.961 mg of 4-phenylbutylamine. This affords 0.281 g of the desired product.

(S)-[1-(4-Phenyl-butylcarbamoyl)-2-(4-sulfoamino-phenyl)-ethyl]-carbamic acid tert-butyl ester: In a similar manner to procedure A, 0.218 g of nitro compound and 50 mg of palladium on carbon is reduced to give the desired product (tlc). This crude aniline compound is treated with 0.236 g of sulfurtrioxide-pyridine complex. Work up and purification yields 0.015 g of product as its ammonium salt. ¹H(D₂O): 7.17-6.92 (m, 9H), 4.08 (m, 1H), 3.05-2.69 (m, 4H), 2.39 (t, 2H, J=7.9 Hz) 1.37-1.24 (m, 4H), 1.19 (s, 9H).

### Example 18

(S)-3-[2-tert-Butoxycarbonlamino-3-(4-sulfoamino-phenyl)-propionylamino]-propionic acid:

(S)-3-[2-tert-Butoxycarbonylamino-3-L-(4-nitro-phenyl)-propionylamino]-propionic acid methyl ester: In a manner similar to example 1, 0.500 g of Boc-L-Phe(4-NO₂)—OH is treated with 0.342 g of NMM, 0.462 g of isobutyl chloroformate, and 0.585 g of β-alanine tert-butyl ester hydrochloride. This affords 0.293 g of the desired product.

(S)-3-[2-tert-Butoxycarbonylamino-3-L-(4-nitro-phenyl)-propionylamino]-propionic acid: 3-[2-tert-Butoxycarbonylamino-3-L-(4-nitro-phenyl)-propionylamino]-propionic acid tert-butyl ester (293 mg) is dissolved in 15 ml methanol:water (1:1) with 0.231 g lithium hydroxide monohydrate. The reaction is stirred at room temperature for 72 hr. The mixture is then diluted with DCM (10 mL) and washed with 1N HCl, and dried over MgSO₄, to afford 0.193 g of the desired product.

(S)-3-[2-tert-Butoxycarbonylamino-3-(4-sulfoamino-phenyl)-propionylamino]-propionic acid: In a similar manner to procedure A, 0.193 g of nitro compound and 50 mg of palladium on carbon is reduced to give the desired product (tlc). This crude aniline compound is treated with 0.242 g of sulfurtrioxide-pyridine complex. Work up and purification yields 0.020 g of product as its ammonium salt. ¹H(D₂O); 7.16-6.97 (q, 4H, J=11.8 Hz), 4.09 (t, 1H, J=6.8 Hz), 3.30-3.14 (m, 2H), 2.95-2.70 (m, 2H), 2.23 (t, 2H, J=10.0 Hz), 1.23 (s, 9H).

### Example 19

(S)-{4-[2-(3-Benzyl-ureido)-2-methylcarbamoyl-ethyl]-phenyl}-sulfamic acid:

(S)-2-(3-Benzyl-ureido)-N-methyl-3-(4-nitro-phenyl)-propionamide: In a manner similar to example 2, 0.150 g of H-L-Phe(4-NO₂)—NMe is treated with 0.123 g of triethylamine, and 0.85 g of benzyl isocyanate. This affords 0.082 g of the desired product.

(S)-{4-[2-(3-Benzyl-ureido)-2-methylcarbamoyl-ethyl]-phenyl}-sulfamic acid: In a similar manner to procedure A, 0.082 g of nitro compound and 50 mg of palladium on carbon is reduced to give the desired product (tlc). This crude aniline compound is treated with 0.110 g of sulfurtrioxide-pyridine complex. Work up and purification yields 0.024 g of product as its ammonium salt. ¹H(D₂O). 7.28-7.18 (m, 3H), 7.06-6.98 (m, 6H), 4.21 (t, 1H, J=7.3 Hz), 4.12 to 4.08 (q, 2H, J=13.3 Hz), 2.96-2.70 (m, 2H), 2.54 (s, 3H).

### Example 20

(S)-(4-{2-3-(2-Methoxy-phenyl)-ureido]-2-methylcarbamoyl-ethyl}-phenyl)-sulfamic acid:

(S)-2-[3-(2-Methoxy-phenyl)-ureidol-N-methyl-3-(4-nitro-phenyl)-propionamide: In a manner similar to example 2, 0.150 g of H-Phe(4-NO₂)—NMe is treated with 0.123 g of triethylamine, and 0.95 g of 2-methoxyphenyl isocyanate. This affords 0.073 g of the desired product.

(S)-(4-{2-[3-(2-Methoxy-phenyl)-ureido]-2-methylcarbamoyl-ethyl}-phenyl)-sulfamic acid: In a similar manner to procedure A, 0.073 g of nitro compound and 50 mg of palladium on carbon is reduced to give the desired product (tlc). This crude aniline compound is treated with 0.094 g of sulfurtrioxide-pyridine complex. Work up and purification yields 0.033 g of product as its ammonium salt. ¹H(D₂O): 7.31-7.28 (m, 1H), 7.02-6.99 (m, 5H), 6.90-6.82 (m, 2H), 4.23 (t, 1H, J=7.0 Hz), 3.66 (s, 3H), 2.94-2.74 (m, 2H), 2.54 (s, 3H).

### Example 21

(S)-[4-(2-Benzenesulfonylamino-2-methylcarbamoyl-ethyl)-phenyl]-sulfamic acid:

(S)-2-Benzenesulfonylamino-N-methyl-3-(4-nitro-phenyl)-propionamide: In a manner similar to example 2, 0.293 g of H-L-Phe(4-NO₂)—NMe is treated with 0.179 g of pyridine, and 0.199 g of benzenesulfonyl chloride. This affords 0.273 g of the desired product.

(S)-[4-(2-Benzenesulfonylamino-2-methylcarbamoyl-ethyl)-phenyl]-sulfamic acid: In a similar manner to procedure A, 0.237 g of nitro compound and 50 mg of palladium on carbon is reduced to give the desired product (tlc). This crude aniline compound is treated with 0.311 g of sulfurtrioxide-pyridine complex. Work up and purification yields 0.085 g of product as its ammonium salt. ¹H(D₂O) 7.55-7.49 (m, 3H), 7.41-7.39 (m, 2H), 6.84 (s, 4H), 3.73 (t, 1H, J=6.8 Hz), 2.86-2.59 (m, 2H), 2.42 (s, 3H).

### Example 22

(S)-{4-[2-(4-Methoxy-benzenesulfonylamino)-2-methylcarbamoyl-ethyl]-phenyl}-sulfamic acid:

(S)-2-(4-Methoxy-benzenesulfonylamino)-N-methyl-3-(4-nitro-phenyl)-propionamde: In a manner similar to example 2, 0.300 g of H-L-Phe(4-NO₂)—NMe is treated with 5 mL pyridine, and 0.262 g of 4-methoxybenzenesulfonyl chloride. This affords 0.086 g of the desired product.

(S)-{4-[2-(4-Methoxy-benzenesulfonylamino)-2-methylcarbamoyl-ethyl]-phenyl}-sulfamic acid: In a similar manner to procedure A, 0.086 g of nitro compound and 25 mg of palladium on carbon is reduced to give the desired product (tlc). This crude aniline compound is treated with 0.105 mg of sulfur trioxide pyridine complex. Work up and purification yields 0.016 g of product as its ammonium salt. ¹H(D₂O): 7.35 and 7.31 (d, 2H, J=7.7 Hz), 6.82 and 6.79 (d, 2H, J=7.7 Hz), 6.76 (s, 4H), 3.74 (s, 3H), 3.64 (t, 1H, J=9.2 Hz), 2.81-2.48 (m, 2H), 2.41 (s, 3H).

### Example 23

(S)-{4-[2-Methylcarbamoyl-2-(naphthalene-1-sulfonylamino)-ethyl]-phenyl}-sulfamic acid:

(S)-N-Methyl-2-(naphthalene-1-sulfonylamino)-3-(4-nitro-phenyl)-propionamide: In a manner similar to example 2, 0.300 g of H-L-Phe(4-NO₂)—NMe is treated with 5 mL pyridine, and 0.287 g of α-napthylsulfonyl chloride. This affords 0.073 g of the desired product.

(S)-{4-[2-Methylcarbamoyl-2-(naphthalene-c-sulfonylamino)-ethyl]-phenyl}-sulfamic acid: In a similar manner to procedure A, 0.073 g of nitro compound and 25 mg of palladium on carbon is reduced to give the desired product (tlc). This crude aniline compound is treated with 0.084 mg of sulfur trioxide pyridine complex. Work up and purification yields 0.018 g of product as its ammonium salt. ¹H(D₂O): 8.07 and 8.04 (d, 1H, J=8.9 Hz), 7.97 and 7.94 (d, 1H, J=9.2 Hz), 7.83 (t, 2H, J=8.0 Hz), 7.46-7.41 (m, 2H), 7.32 (t, 1H, J=8.8 Hz), 6.45 and 6.42 (d, 2H, J=9.4 Hz), 6.29 and 6.27 (d, 2H, J=7.5 Hz), 3.64-3.58 (q, 1H, J=5.5 Hz), 2.71-2.31 (m, 2H), 2.36 (s, 3H).

### Example 24

(S)-[1-(Benzyl-methyl-carbamoyl)-2-(4-sulfoamino-phenyl)-ethyl]-carbamic acid tert-butyl ester:

(S)-[1-(Benzyl-methyl-carbamoyl)-2-(4-nitro-phenyl)-ethyl]-carbamic acid tert-butyl ester: L-Boc-Phe-(4-NO₂)—OH (0.390 g) and 0.203 g 1-Hydroxybenzatriazole monohydrate are combined in 12 mL DMF and cooled in an ice bath. EDCI (0.575 g), and 0.39 mL N-methylbenzyl amine is added. The reaction is then allowed to warm to ambient temperature. After 18 hours the reaction is diluted with EtOAc, washed twice with 1N HCl, and dried over sodium sulfate. Chromatography affords 0.910 g of a white solid.

(S)-[2-(4-Amino-phenyl)-1-(benzyl-methyl-carbamoyl)-ethyl]-carbamic acid tert-butyl ester:

In a manner similar to procedure A, 0.900 g of nitro compound is combined with 10 mL MeOH and 0.150 g 10% Pd/C. Reduction and purification by flash chromatography affords 0.427 g of the desired product.

(S)-[1-(Benzyl-methyl-carbamoyl)-2-(4-sulfoamino-phenyl)-ethyl]-carbamic acid tert-butyl ester:

In a manner similar to procedure A, 0.427 g of the aniline is combined with 10 mL pyridine and 0.530 g of pyridine-SO₃ complex pressure. After purification 0.324 g of product is isolated. ¹H NMR (DMSO) Mixture of rotational isomers. 7.73 (1H, s), 7.68 (1H, s), 7.38-6.82 (9H, m), 6.79 (1H, d), 4.604.51 (1H, m), 4.41 (s, 2H), 4.36 (s, 2H), 2.84 (s, 3H), 2.79-2.62 (m, 2H), 1.38 (s, 9H), 1.32 (s, 9H).

### Example 25

(S)-[1-(2-Methyl-5-phenyl-2H-pyrazol-3-ylcarbamoyl)-2-(4-sulfoamino-phenyl)-ethyl]carbamic acid tert-butyl ester:

(S)-[1-(2-Methyl-5-phenyl-2H-pyrazol-3-ylcarbamoyl)-2-(4-nitro-phenyl)-ethyl]-carbamic acid tert-butyl ester:

In a manner similar to procedure A, 0.930 g of Boc-Phe-(4-NO₂)—OH is treated with 0.36 mL of N-Methylmorpholine, 0.39 mL isobutylchloroformate, and 0.55 mL of 2-methyl-5-phenyl-2H-pyrazol-3-ylamine. Work up and chromatography affords the desired product.

(S)-[2-(4-Amino-phenyl)-1-(2-methyl-5-phenyl-2H-pyrazol-3-ylcarbamoyl)-ethyl]-carbamic acid tert-butyl ester:

In a manner similar to procedure A, 0.500 g of the nitro compound is dissolved in MeOH and 0.150 g 10% Pd/C is added. The reaction is placed under H₂ atmosphere. Workup and chromatography yields 0.388 g of an off-white solid.

(S)-[1-(2-Methyl-5-phenyl-2H-pyrazol-3-ylcarbamoyl)-2-(4-sulfoamino-phenyl)-ethyl]carbamic acid tert-butyl ester:

In manner similar to procedure A, 0.388 g of aniline is dissolved in 8 mL pyridine and treated with 0.410 g of pyridine-SO₃ complex. Work up and purification affords 0.169 g of product as its ammonium salt. ¹H NMR (D₂O) Mixture of rotational isomers. 7.61-7.52 (m, 2H), 7.40-7.22 (m, 3H), 7.15-7.02 (m, 4H), 6.41-6.29 (m, 1H), 4.394.29 (m, 1H), 3.42-3.25 (m, 3H), 2.99-2.82 (m, 2H), 1.31 (s, 9H).

### Example 26

(S)-[1-Phenylcarbamoyl-2-(4-sulfoamino-phenyl)-ethyl]-carbamic acid tert-butyl ester:

(S)-[2-(4-Nitro-phenyl)-1-phenylcarbamoyl-ethyl]-carbamic acid tert-butyl ester:

In a manner similar to procedure A, 0.930 g of Boc-Phe-(4-NO₂)—OH is treated with 0.36 mL of N-Methylmorpholine, 0.39 mL isobutylchloroformate, and 0.55 mL of aniline is added. Work up and chromatography affords 1.143 g of the desired product.

(S)-[2-(4-Amino-phenyl)-1-phenylcarbamoyl-ethyl]-carbamic acid tert-butyl ester

In a manner similar to procedure A, 0.500 g of the nitro compound is dissolved in MeOH and 0.150 g 10% Pd/C is added. The reaction is placed under H₂ atmosphere. Workup yields 0.373 g of the desired product.

(S)-[1-Phenylcarbamoyl-2-(4-sulfoamino-phenyl)-ethyl]-carbamic acid tert-butyl ester

In manner similar to procedure A, 0.373 g of aniline is dissolved in 5 mL pyridine and treated with 0.501 g of pyridine-SO₃ complex. Work up and purification affords 0.045 g of product as its ammonium salt. ¹H NMR (D₂O). 7.36-7.24 (m, 1H), 7.21-7.12 (m, 5H), 7.11-7.02 (m, 3H), 4.31-4.11 (m, 1H), 3.00 (d, 2H, J=10.4 Hz), 1.32 (s, 9H).

### Example 27

(S)-[1-Dibenzylcarbamoyl-2-(4-sulfoamino-phenyl)-ethyl]-carbamic acid tert-butyl ester:

(S)-[1-Dibenzylcarbamoyl-2-(4-nitro-phenyl)-ethyl]-carbamic acid tert-butyl ester:

In a manner similar to example 24, 0.930 g Boc-Phe-(4-NO₂)—OH and 203 g 1-Hydroxybenzatriazole monohydrate are combined in 12 mL DMF. EDCI (0.575 g) and 0.57 mL dibenzyl amine are then added. Work up and purification by chromatography affords 0.726 g of a white solid.

(S)-[2-(4-Amino-phenyl)-1-dibenzylcarbamoyl-ethyl]-carbamnic acid tert-butyl ester:

In a manner similar to procedure A, 0.726 g of nitro compound and 0.120 g of 10% Pd/C are combined with 15 mL MeOH. Work up and purification by flash chromatography affords 0.172 g of a white solid.

(S)-[1-Dibenzylcarbamoyl-2-(4-sulfoamino-phenyl)-ethyl]-carbamic acid tert-butyl ester:

In a manner similar to procedure A, 0.172 g of amine is dissolved in 5 mL pyridine and treated with 0.177 g of pyridine-SO₃ complex Work up and purification gives 0.084 g of product as its ammonium salt. ¹H NMR (D₂O) Mixture of rotational isomers. 7.35-7.23 (m, 5H), 7.11-6.92 (m, 9H), 4.60-4.32 (m, 1H), 2.83 (d, 2H, J=10.4 Hz), 1.28 (s, 9H), 1.02 (s, 9H).

### Example 28

(S)-4-[1-Methylcarbamoyl-2-(4-sulfoamino-phenyl)-ethylcarbamoyl]-piperidine-1-carboxylic acid tert-butyl ester:

(S)-4-[1-Methylcarbamoyl-2-(4-nitro-phenyl)-ethylcarbamoyl]-piperidine-1-carboxylic acid tert-butyl ester: H-L-Phe(4-NO₂)—NMe (0.300 g) is dissolved in 10 mL DCM and treated with 0.130 g triethylamine, 0.245 g EDCI, and 0.305 g 1-BOC-piperidine-4-carboxylic acid. After 18 hours, the mixture is partitioned between 0.1N HCl and DCM. The organic layer is dried and purified by flash chromatography to yield 0.137 g of product.

(S)-4-[1-Methylcarbamoyl-2-(4-sulfoamino-phenyl)-ethylcarbamoyl]-piperidine-lcarboxylic acid tert-butyl ester: In a similar manner to procedure A, 0.137 g of nitro compound and 25 mg of palladium on carbon is reduced to give the desired product (tlc). This crude aniline compound is treated with 0.150 g of sulfur trioxide pyridine complex. Work up and purification yields 0.015 g of product as its ammonium salt. ¹H(D₂O): 7.03-6.95 (q, 4H, J=8.2 Hz), 4.32 (t, 1H, J=7.9 Hz), 3.81 (m, 2H), 2.95-2.62 (m, 4H) 2.49 (s, 3H), 2.28 (m, 1H) 1.52 (m, 4H), 1.27 (s, 9H)

### Example 29

(S)-[4-(2-Benzovlamino-2-methylcarbamoyl-ethyl)-phenyl]-sulfamic acid:

(S)-N-[1-Methylcarbamoyl-2-(4-nitro-phenyl)-ethyl]-benzamide: In a manner similar to example 2, 0.175 g of H-L-Phe(4-NO₂)—NMe is treated with 0.143 g triethylamine, and 0.104 g benzoyl chloride to provide 0.110 g of product.

(S)-[4-(2-Benzoylamino-2-methylcarbamoyl-ethyl)-phenyl]-sulfamic acid: In a similar manner to procedure A, 0.110 g of nitro compound is combined with 0.050 g of palladium on carbon and reduced to give the desired product (tlc). This crude aniline compound is treated with 0.277 g of sulfur trioxide pyridine complex. Work up and purification yields 0.034 g of product as its ammonium salt. ¹H(D₂O): 7.59-7.32 (m, 5H), 7.16-6.99 (m, 4H), 4.53 (t, 1H, J=8.9 Hz), 3.082.90 (m, 2H), 2.55 (s, 3H).

### Example 30

(S)-[1-Dimethylcarbamoyl-2-(4-sulfoamino-phenyl)-ethyl]-carbamic acid tert-butyl ester:

(S)-[1-Dimethylcarbamoyl-2-(4-nitro-phenyl)-ethyl]-carbamic acid tert-butyl ester

In a manner similar to example 1, 0.300 g of Boc-L-Phe(4-NO₂)—OH is treated with 0.108 grams of NMM, 0.132 g of isobutyl chloroformate, and 0.967 mL of dimethylamine solution (2.0M in THF). This affords 0.316 g of product.

(S)-[1-Dimethylcarbamoyl-2-(4-sulfoamino-phenyl)-ethyl]-carbamic acid tert-butyl ester: In a similar manner to procedure A, 0.316 g of nitro compound is combined with 0.100 g of palladium on carbon and reduced to give the desired product (tlc). This crude aniline compound is treated with 0.447 g of sulfur trioxide pyridine complex. Work up and purification yields 0.168 g of product as its ammonium salt. ¹H(D₂O): 7.05-6.97 (q, 4H, J=8.1 Hz), 4.53 (t, 1H, J=7.9 Hz), 2.73-2.57 (m, 8H), 1.21 (s, 9H)

### Example 31

(S)-(4-{2-Methylcarbamoyl-2-[(Pyridine-3-carbonyl)-amino]-ethyl}-phenyl)-sulfamic acid:

(S)-N-[1-Methylcarbamoyl-2-(4-nitro-phenyl)-ethyl]-nicotinamide: In a manner similar to example 2, 0.300 g of H-L-Phe(4-NO₂)—NMe is treated with 0.387 g of triethylamine, and 0.226 g of nicotinoyl chloride.

(S)-(4-{2-Methylcarbamoyl-2-[(pyridine-3-carbonyl)-amino]-ethyl}-phenyl)-sulfamic acid: In a similar manner to procedure A, 0.725 g of nitro compound is combined with 0.100 g of palladium on carbon and reduced to give the desired product(tlc). This crude aniline compound is treated with 1.054 g of sulfur trioxide pyridine complex. Work up and purification yields 0.025 g of product as its ammonium salt. ¹H(D₂O): 8.57 (d, 1H, J=2.3 Hz), 8.47 (d, 1H, J=5.6 Hz), 8.88-7.85 (d, 1H, J=9.2 Hz), 7.35-7.31 (m, 1H), 7.07-6.95 (q, 4H, J=9.5 Hz), 4.50 (t, 1H, J=8.1 Hz), 3.04-2.85 (m, 2H), 2.51 (s, 3H).

### Example 32

(S)-[4-(2-Methylcarbamovl-2-phenylacetylamino-ethyl)-phenyl]-sulfamic acid:

(S)-N-Methyl-3-(4-nitro-phenyl)-2-phenylacetylaniino-propionamide: In a manner similar to example 2, 0.300 g of H-L-Phe(4-NO₂)—NMe is treated with 0.258 g of triethylamine, and 0.242 g of phenylacetyl chloride. This affords 0.513 g of product.

(S)-[4-(2-Methylcarbamoyl-2-phenylacetylamino-ethyl)-phenyl]-sulfaniic acid: In a similar manner to procedure A, 0.513 g of nitro compound is combined with 0.075 g of palladium on carbon and reduced to give the desired product(tlc). This crude aniline compound is treated with 0.716 g of sulfur trioxide pyridine complex. Work up and purification yields 0.030 g of product as its ammonium salt. ¹H(D₂O) 7.19-7.13 (m, 3H), 6.92-6.89 (m, 6H), 4.32 (t, 1H, J=6.7 Hz), 3.423.30 (q, 2H, J=8.9 Hz), 2.93-2.64 (m, 2H), 2.50 (s, 3H).

### Example 33

(S)(4-{2-Methylcarbamoyl-2-[(naphthalene-1-carbonyl)-amino]-ethyl}-phenyl)-sulfamic acid:

(S)-Naphthalene-2-carboxylic acid [1-methylcarbamoyl-2-(4-nitro-phenyl)-ethyl]-amide: In a manner similar to example 2, 0.300 g of H-L-Phe(4-NO₂)—NMe is treated with 0.258 g of triethylamine, and 0.242 g of 2-napthoyl chloride. This affords 0.423 g of product.

(S)-(4-{2-Methylcarbamoyl-2-[(naphthalene-1-carbonyl)-amino]-ethyl}-phenyl)-sulfamic acid: In a similar manner to procedure A, 0.423 g of nitro compound is combined with 0.075 g of palladium on carbon and reduced to give the desired product(tlc). This crude aniline compound is treated with 0.535 g of sulfur trioxide pyridine complex. Work up and purification yields 0.026 g of product as its ammonium salt. ¹H(D₂O): 8.26 (s,1H), 7.99 (s, 1H), 7.83-7.76 (m, 3H), 7.49-7.44 (m, 4H), 7.13-7.10 (d, 1H, J=9.5 Hz), 7.02-6.99 (d, 1H, J=9.5 Hz), 4.59 (m, 1H), 3.032.96 (m, 2H), 2.55 (s, 3H).

### Example 34

(S)-{4-[2-(Cycloventanecarbonyl-amino)-2-methylcarbamoyl-ethyl]-phenyl}-sulfamic acid:

(S)-Cyclopentanecarboxylic acid [1-methylcarbamoyl-2-(4-nitro-phenyl)-ethyl]-amide: In a manner similar to example 2, 0.300 g of H-L-Phe(4-NO₂)—NMe is treated with 0.258 g of triethylamine, and 0.168 g of cyclopentanecarbonyl chloride. This affords 0.359 g of product.

(S)-[4-[2-(Cyclopentanecarbonyl-amino)-2-methylcarbamoyl-ethyl]-phenyl]-sulfamic acid: In a similar manner to procedure A, 0.359 g of nitro compound is combined with 0.050 g of palladium on carbon and reduced to give the desired product(tlc). This crude aniline compound is treated with 0.537 g of sulfur trioxide pyridine complex. Work up and purification yields 0.032 g of product as its ammonium salt. ¹H(D₂O) 7.06-6.98 (q, 4H, J=9.3 Hz), 4.34 (t, 1H, J=9.0 Hz), 2.97-2.77 (m, 2H), 2.52-2.49 (m, 4H), 2.55 (s, 3H), 1.67 (m, 2H), 1.44 (m, 5H), 1.25-1.21 (m, 1H).

### Example 35

(S)-(4-[2-Benzylcarbamoyl-2-[2-(4-propyl-phenyl)-acetylamino]-ethyl]-phenyl)-sulfamic acid:

(S)-N-Benzyl-3-(4-nitro-phenyl)-2-[2-(4-propyl-phenyl)-acetylamino]-propionamide: (S)-[1-Benzylcarbamoyl-2-(4-nitrophenyl)-ethyl]-carbamic acid tert-butyl ester (0.971 g) is combined with 11 mL 4M HCl/dioxane. After 1.5 the reaction is concentrated to afford 0.914 g of the desired product.

(S)-3-(4-Amino-phenyl)-N-benzyl-2-[2-(4-propyl-phenyl)-acetylamino]-propionamide: In a manner similar to example 2, 0.300 g of the amine is combined with 0.37 mL triethylamine and 0.16 mL 4-propylbenzylchloride. Workup and chromatography affords 0.333 g of product.

(S)-(4-{2-Benzylcarbamoyl-2-[2-(4-propyl-phenyl)-acetylamino]-ethyl}-phenyl)-sulfamic acid: In a manner similar to procedure A, 0.325 g of nitro compound is combined with 0.260 g of Pd/C and 25 mL THF/EtOAc and reduced. Workup affords 0.257 g of the desired product. In a manner similar to procedure A, 0.257 g of amine is combined with 0.319 g of SO₃-pyridine in 5 mL pyridine. Workup and purification affords 0.102 g of product. ¹H(DMSO-d₆): 8.53 (1H, t, J=5.7 Hz), 8.39 (1H, d, J=8.4 Hz), 7.75 (2H, d, J=8.4 Hz), 7.35-6.90 (9H, m), 4.64 (1H, m), 4.31 (1H, d, J=5.7 Hz), 2.98-2.83 (2H, m), 2.60 (2H, t, 7.8 Hz), 1.61 (2H, q, J=7.5), 0.89 (3H, t, J=10.5 Hz).

### Example 36

(S)-(4-{2-[3-(3-Acetylsulfamoyl-pheny)-propionylamino]-2-methylcarbamoyl-ethyl}-phenyl)-sulfamic acid:

(S)-2-[3-(3-Acetylsulfamoyl-phenyl)-propionylamino]-N-methyl-3-4-nitro-phenyl)propionamide: 3-(3-Acetylsulfamoyl-phenyl)-propionic acid (0.214 g, 0.82 mmol) and HOBt (0.150 g) are dissolved in DMF (1.5 mL) and cooled to 0° C. To this stirring solution is added EDCI (0.188 g) and the resulting slurry is stirred at 0° C. for 45 min at which point the reaction is homogeneous. To this solution is added (S)-2-amino-N-methyl-3-(4-nitro-phenyl)-propionamide hydrochloride (0.224 g, 0.82 mmol) as a solution in DMF (2 mL) and DiPEA (0.300 mL). The resulting solution is allowed to slowly warm to room temperature over a period of 18 h. The reaction solution is diluted with ethyl acetate (40 mL) and washed with 1N HCl (3×20 mL), brine (1×25 mL) and dried over sodium sulfate. The crude material is purified by flash column chromatography on silica gel eluting with 8:1 chloroform/methanol to give an off-white solid (0.188 g).

(S)-(4-{2-[3-(3-Acetylsulfamoyl-pheny)-propionylamino]-2-methylcarbamoyl-ethyl}-phenyl)-sulfamic acid: In a similar manner to procedure A, 0.188 g of nitro compound and 100 mg of palladium on carbon is reduced to give the desired product (tlc). This crude aniline compound is treated with 0.134 g of sulfurtrioxide-pyridine complex. Work up and purification yields 0.085 g of product as its ammonium salt. ¹HNMR (300 MHz, D₂O) δ 7.65-7.59 (m, 2H), 7.38-7.23 (m, 2H), 6.97-6.88 (m, 4H), 4.21 (dd, J=7.4, 6.5 Hz, 1H), 2.87-2.64 (m, 4H), 2.48 (m, 5H), 1.83 (s, 3H).

### Examples 37-38

The following chemical formula along with Table 2 shows the structure of compounds made according to the description in Examples 37-38 below:

TABLE 2

| | |
|---|---|
| | Formula (III) |
| 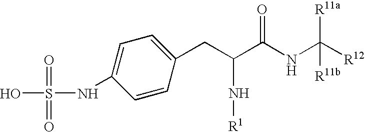 | |

| EXAMPLE | * | R¹ | R^{11a} | R^{11b} | R¹² |
|---|---|---|---|---|---|
| 37 | S | 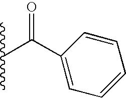 | 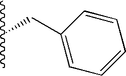 | 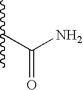 | H |
| 38 | S | 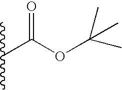 | 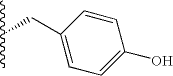 | 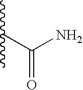 | H |

### Example 37

(S)-{4-[2-Benzovlamino-2-(1-carbamoyl-2-(S)-phenyl-ethylcarbamoyl)-ethyl]-phenyl}-sulfamic acid:

(S)-[1-[1-Carbamoyl-2-phenyl-ethylcarbamoyl]-2-(S)-(4-nitrophenyl)-ethyl]-carbamic acid tert-butyl ester: In a manner similar to example 1, 0.400 g of Boc-L-Phe(4-NO₂)—OH is treated with 143 mg of NMM, 194 mg of isobutyl chloroformate, and 255 mg of L-Phenylalaninamide. This affords the product as white solid.

(S)-N-[1-(1-Carbamoyl-2-phenyl-ethylcarbamoyl)-2-(S)-(4-nitro-phenyl)-ethyl]-benzamide: In a manner similar to procedure B, 0.300 g of the compound is treated with 5 mL of 4M HCl/dioxane. The crude product is then treated with 0.140 g triethylamine and 0.102 g of benzoyl chloride. This affords 0.048 g of product

(S)-{4-[2-Benzoylamino-2-(1-carbamoyl-2-(S)-phenyl-ethylcarbamoyl)-ethyl]-phenyl}-sulfamic acid: In a similar manner to procedure A, 0.048 g of nitro compound and 40 mg of palladium on carbon is reduced to give the desired product (tlc). This crude aniline compound is treated with 0.050 g of sulfurtrioxide-pyridine complex. Work up and purification yields 0.018 g of product as its ammonium salt. ¹H(CD₃OD): 6.11-6.04 (m, 3H), 5.98-5.93 (m, 2H), 5.68-5.54 (m, 9H) 3.20 (m, 1H), 3.02 (m, 1H) 1.79-1.34 (m, 4H).

### Example 38

(S)-[1-[1-Carbamoyl-2-(4-hydroxy-phenyl)-ethylcarbamoyl]-2(S)-(4-sulfoamino-phenyl)-ethyl]-carbamic acid tert-butyl ester:

(S)-[1-[1-Carbamoyl-2-(4-hydroxy-phenyl)-ethylcarbamoyl]-2(S)-(4-nitrophenyl)-ethyl]carbamic acid tert-butyl ester: In a manner similar to example 1, 1.56 g of Boc-L-Phe(4-NO₂)—OH is treated with 0.559 g of NMM, 0.755 g of isobutyl chloroformate, and 1.09 grams of L-tyrosinamide. This affords 0.166 g of the desired product.

(S)-[1-[1-Carbamoyl-2-(4-hydroxy-phenyl)-ethylcarbamoyl]-2(S)-(4-sulfoamino-phenyl)-ethyl]-carbamic acid tert-butyl ester: In a similar manner to procedure A, 0.116 g of nitro compound and 60 mg of palladium on carbon is reduced to give the desired product (tlc). This crude aniline compound is treated with 0.117 g of sulfurtrioxide-pyridine complex. Work up and purification yields 0.011 g of product as its ammonium salt. ¹H(D₂O): 7.07-6.97 (m, 6H), 6.78 (d, 2H, J=9.3 Hz), 4.39 (t, 1H, J=6.5 Hz), 4.08 (t, 1H, J=8.1 Hz), 2.99-2.67 (m, 4H), 1.23 (s, 9H).

### Examples 39-54

The following chemical formula along with Table 3 shows the structure of compounds made according to the description in Examples 39-54 below:

TABLE 3

| | |
|---|---|
| | Formula (IV) |
| 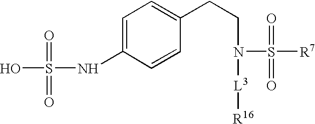 | |

| EXAMPLE | L³ | R⁷ | R¹⁶ |
|---|---|---|---|
| 39 | —CO₂— | 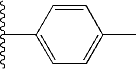 | 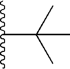 |
| 40 | Covalent bond | 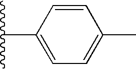 | 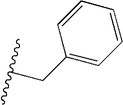 |
| 41 | Covalent bond | 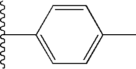 | 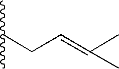 |
| 42 | Covalent bond | 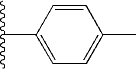 | 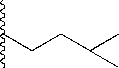 |
| 43 | Covalent bond | 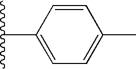 | 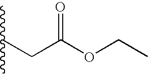 |
| 44 | Covalent bond | 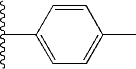 | 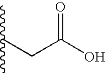 |
| 45 | —CO— | 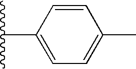 | 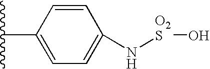 |
| 46 | —CO— | 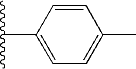 | 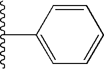 |
| 47 | —CO₂— | 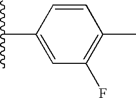 | 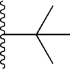 |
| 48 | —CO₂— | 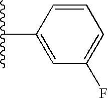 | 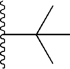 |
| 49 | —CO₂— | 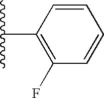 | 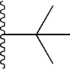 |
| 50 | Covalent bond | 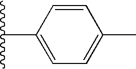 | H |
| 51 | Covalent bond | 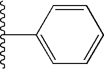 | H |
| 52 | Covalent bond | —CH₃ | H |
| 53 | Covalent bond | 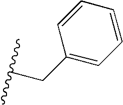 | H |
| 54 | Covalent bond | 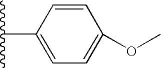 | H |

### Example 39

[4-(2-{(tert-Butoxycarbonyl)[(4-methylphenyl)sulfonyl]amino}ethyl)phenyl]sulfamic acid: 4-Methyl-N-[2-(4-nitro-phenyl)-ethyl]-benzenesulfonamide: A solution of 3.0 g of 4-nitrophenylethylamine hydrochloride, 20.6 mL triethylamine in 120 mL THF is treated with 2.81 g of p-toluenesulfonyl chloride. The reaction is stirred for 65 h, concentrated, and redissolved in CH₂Cl₂. The solution is washed with water and saturated sodium bicarbonate solution and then dried. Trituration with methanol and washing with ether gives 3.47 g of desired product.

tert-Butyl[(4-methylphenyl)sulfonyl][2-(4-nitrophenyl)ethyl] carbamate: The compound from above (0.300 g) is treated with 0.0057 g DMAP and 0.245 g di-tert-butyl in 25 mL methylene chloride and stirred overnight. The solution is washed with water. Hexane is added to the organic phase and the solution is extracted with water. The aqueous phase is concentrated and purified by flash chromatography to afford 0.303 g of the desired product.

tert-Butyl[2-(4-aniinophenyl)ethyl][(4-methylphenyl)sulfonyl] carbamate:

In a manner similar to procedure A, 0.301 g of nitro compound and 0.018 of 10% Pd/C are combined in 5 mL EtOAc and 5 mL methanol under H₂ at atmospheric pressure. Work up affords 0.275 g of desired product.

[4-(2-{(tert-Butoxycarbonyl)[(4-methylphenyl)sulfonyl]amino}ethyl)phenyl]sulfamic acid: In a manner similar to procedure A, 0.276 g of amine in 8 mL pyridine is treated 0.337 of pyridine-sulfurtrioxide. Work up and purification affords 0.111 g of product as its ammonium salt. ¹H NMR (D₂O) δ 7.62 (d, J=5 Hz, 2H); 7.40 (d, J=6 Hz, 2H); 6.95 (m, 4H); 3.87 (t, J=14 Hz, 2H); 2.80 (d, J=6 Hz, 2H); 2.36 (s, 3H); 1.22 (s, 9H).

### Example 40

(4-{2-[Benzyl-(toluene-4-sulfonyl)-amino]-ethyl}-phenyl sulfamic acid:

N-Benzyl-4-methyl-N-[2-(4-nitro-phenyl)-ethyl]-benzenesulfonamide: A suspension of 0.0387 g of sodium hydride (60% oil dispersion) in 2 mL anhydrous DMF is treated with a solution of 0.300 g 4-Methyl-N-[2-(4-nitro-phenyl)-ethyl]-benzenesulfonamide in 4 mL DMF. After 15 min, after 0.11 mL benzyl bromide 2 mL DMF is added dropwise. The reaction is stirred for 3 hours, then quenched with water. The solution was extracted with ether and the combined organic extracts are washed with brine and dried over sodium sulfate. Purification by flash chromatography gives 0.288 g of desired product.

N-[2-(4-Amino-phenyl)-ethyl]-N-benzyl-4-methyl-benzenesulfonamide: In a manner similar to procedure B, 0.288 g of the nitro compound is combined with 0.632 g of tin chloride dihydrate in 10 mL ethanol. Work up affords 0.262 g of desired product.

(4-{2-[Benzyl-(toluene-4-sulfonyl)-amino]-ethyl}-phenyl sulfamic acid: In a manner similar to procedure A, 0.262 g of the amine is combined with 8 mL pyridine and 0.329 g sulfurtrioxide-pyridine. Work up and purification affords 0.118 g of the desired product. ¹H NMR (D₂O): δ 7.45 (d, J=8 Hz, 2H); 7.16-7.20 (m, 5H); 7.07-7.09 (m, 2H); 6.85 (d, J=8 Hz, 2H); 6.70 (d, J=8 Hz, 2H); 4.13 (s, 2H); 3.09 (t, J=14 Hz, 2H); 2.36 (t, J=14 Hz, 2H); 2.22 (s, 3H).

### Example 41

(4-{2-d(3-Methal-but-2-enyl)-(toluene-4-sulfonyl)-amino]-ethyl}-phenyl)-sulfamic acid:

4-Methyl-N-(3-methyl-but-2-enyl)-N-[2-(4-nitro-phenyl)-ethyl-benzene-sulfon amide: In a manner similar to example 40, 0.600 g of 4-Methyl-N-[2-(4-nitro-phenyl)-ethyl]benzenesulfonamide is treated with 0.073 g NaH (60% dispersion) and 0.266 g 4-bromo-2-methyl2-butene to afford 0.434 g of the desired product after workup and chromatography.

N-[2-(4-Amino-phenyl)-ethyl]-4-methyl-N-(3-methyl-but-2-enyl)-benzenesulfonamide:

Nitro compound (0.214 g), 0.400 g indium powder, and 1 mL saturated ammonium chloride are combined in 6 mL ethanol combined and heated to reflux. After 4 hours, the reaction is diluted with water and filtered through celite, washing well with water and then EtOAc. The filtrate is adjusted to pH=10 with 1 N NaOH and extracted with EtOAc. The combined extracts are washed with brine and dried to provide 0.162 g of the desired product.

(4-{2-[(3-Methyl-but-2-enyl)-(toluene-4-sulfonyl)-amino]-ethyl}-phenyl)-sulfamic acid:

In a manner similar to procedure A, 0.262 g of the amine is combined with 0.329 g sulfurtrioxide-pyridine in 5 mL pyridine. Work up and purification affords 0.148 g of the desired product as its ammonium salt. ¹H NMR (D₂O): δ 7.49 (d, J=6 Hz, 2H); 7.27 (d, J=7 Hz, 2H); 6.95-7.02 (m, 4H); 4.82 (s, 1H); 3.67 (d, J=7.0 Hz, 2H); 3.26 (t, J=14 Hz, 2H); 2.66 (t, J=14 Hz, 2H); 2.29 (s, 3H); 1.49 (d, J=11 Hz, 6H).

### Example 42

(4-{2-(3-Methyl-butyl)-(toluene-4-sulfonyl)-amino]-ethyl}-phenyl)-sulfamic acid:

N-[2-(4-Amino-phenyl)-ethyl]-4-methyl-N-(3-methyl-butyl)-benzenesulfonamide:

In a manner similar to procedure A, 0.198 g 4-Methyl-N-(3-methyl-but-2-enyl)-N-[2-(4-nitrophenyl)-ethyl]-benzene-sulfonamide is combined with 0.030 g Pd/C in 20 mL MeOH and reduced to afford 0.192 g of the desired product.

(4-{2-[(3-Methyl-butyl)-(toluene-4-sulfonyl)-amino]-ethyl}-phenyl)-sulfanic acid:

In a manner similar to procedure A, 0.192 g of the amine is treated with 0.254 g of sulfurtrioxide-pyridine in 5 mL pyridine. Workup and purification affords 0.161 g of the desired product as its ammonium salt. ¹H NMR (D₂O): δ 7.50 (d, J=7 Hz, 2H); 7.15 (d, J=7 Hz, 2H); 6.98 (q, J=17 Hz, 4H); 3.29 (t, J=14 Hz, 4H); 3.06 (t, J=15 Hz, 2H); 2.64 (t, J=14 Hz, 2H); 2.29 (s, 3H); 1.26-1.35 (m, 1H); 1.21-1.24 (m, 2H); 0.70 (d, J=7 Hz, 6H).

### Example 43

[[2-(4-Sulfoamino-phenyl)-ethyl]-(toluene-4-sulfonyl)-amino]-acetic acid ethyl ester:

[[2-(4-Nitro-phenyl)-ethyl]-(toluene-4-sulfonyl)-amino]-acetic acid ethyl ester:

In a manner similar to example 40, 0.600 g 4-Methyl-N-[2-(4-nitro-phenyl)-ethyl]benzenesulfonamide is combined with 0.094 g sodium hydride (60% dispersion) in 5 mL THF. Ethyl bromoacetate (0.313 g) is added and after workup 0.798 g of desired product is isolated.

[[2-(4-Aniino-phenyl)-ethyl]-(toluene-4-sulfonyl)-amino]-acetic acid ethyl ester:

In a manner similar to procedure A, 0.381 g of nitro compound is combined with 0.023 g Pd/C in 10 mL EtOAc/MeOH and reduced to afford 0.341 g of the desired product.

[[2-(4-Sulfoamino-phenyl)-ethyl]-(toluene-4-sulfonyl)-amino]-acetic acid ethyl ester:

In a manner similar to procedure A, 0.341 g of the amine is treated with 0.432 g of sulfurtrioxide-pyridine in 5 mL pyridine. Workup and purification affords 0.177 g of the desired product as its ammonium salt. ¹H NMR (D₂O-d₂): δ 7.46 (d, J=8 Hz, 2H); 7.24 (d, J=8 Hz, 2H); 6.91 (s, 4H); 3.954.01 (m, 4H); 3.37 (t, J=14 Hz, 2H); 2.61 (t, J=14 Hz, 2H); 2.29 (s, 3H); 1.07 (t, J=14 Hz, 3H).

### Example 44

[[2-(4-Sulfoamino-phenyl)-ethyl]-(toluene-4-sulfonyl)-amino]-acetic acid:

[[2-(4-Nitro-phenyl)-ethyl]-(toluene-4-sulfonyl)-amino]-acetic acid:

[[2-(4-Nitro-phenyl)-ethyl]-(toluene-4-sulfonyl)-amino]-acetic acid ethyl ester (0.471 g) is combined with 15 mL MeOH, 5 mL water and 0.123 g potassium carbonate and refluxed. After 4 hours, the reaction is cooled to room temperature and the MeOH removed by evaporation. The solution is washed with hexane and then acidified. The solution is extracted with chloroform and the combine organics are dried to afford 0.285 g of desired product.

[[2-(4-Amino-phenyl)-ethyl]-(toluene-4-sulfonyl)-amino]-acetic acid:

In a manner similar to procedure A, 0.285 g of nitro compound is combined with 0.017 g Pd/C in 10 mL EtOAc/MeOH and reduced to afford 0.271 g of the desired product.

[[2-(4-Sulfoamino-phenyl)-ethyl]-(toluene-4-sulfonyl)-amino]-acetic acid:

In a manner similar to procedure A, 0.271 g of the amine is treated with 0.371 g of sulfurtrioxide-pyridine in 5 mL pyridine. Workup and purification affords 0.023 g of the desired product as its ammonium salt. ¹H NMR (D₂O): δ 7.48 (d, J=8 Hz, 2H); 7.24 (d, J=8 Hz, 2H); 6.91 (q, J=14 Hz, 4H); 3.68 (s, 2H); 3.36 (t, J=15 Hz, 2H); 2.57 (t, J=15 Hz, 2H); 2.29 (s, 3H).

### Example 45

[4-(2-{[(4-Methylphenyl)sulfonyl][4-(sulfoamino)benzoyl]amino}ethyl)phenyl]sulfamic acid:

4-Methyl-N-(4-nitro-benzoyl)-N-[2-(4-nitro-phenyl)-ethyl]-benzenesulfonamide:

In a manner similar to example 40, 0.300 g 4-Methyl-N-[2-(4-nitro-phenyl)-ethyl]-benzenesulfonamide is combined with 0.047 g sodium hydride (60% dispersion) in 5 mL THF and treated with 0.173 g p-nitrobenzoylchloride. Workup and purification affords 0.205 g of the desired product.

N-(4-Amino-benzoyl)-N-[2-(4-amino-phenyl)-ethyl]-4-methyl-benzenesulfonamide:

In a manner similar to procedure A, 0.207 g of nitro compound is combined with 0.032 g Pd/C in 10 mL EtOAc/MeOH and reduced to afford 0.137 g of the desired product.

[4-(2-{[(4-Methylphenyl)sulfonyl][4-(sulfoamino)benzoyl]amino}ethyl)phenylsulfamic acid:

In a manner similar to procedure A, 0.137 g of the amine is treated with 0.320 g of sulfurtrioxide-pyridine in 5 mL pyridine. Workup and purification affords 0.106 g of the desired product as its ammonium salt. ¹H NMR (D₂O): δ 7.55 (d, J=8 Hz, 2H); 7.32 (d, J=8 Hz, 2H); 6.91 (s, 4H); 6.84 (d, J=8 Hz, 2H); 6.76 (d, J=8 Hz, 2H); 4.21 (t, J=11 Hz, 2H); 2.74 (t, J=11 Hz, 2H); 2.30 (s, 3H).

### Example 46

(4-{2-[Benzovl-(toluene-4-sulfonyl)-amino]-ethyl}-phenyl sulfamic acid:

N-Benzoyl-4-methyl-N-[2-(4-nitro-phenyl)-ethyl-benzenesulfonamide:

In a manner similar to example 40, 0.300 g 4-Methyl-N-[2-(4-nitro-phenyl)-ethyl]-benzenesulfonamide is combined with 0.047 g sodium hydride (60% dispersion) in 5 mL THF and treated with 0.131 g p-nitrobenzoylchloride. Workup and purification affords 0.245 g of the desired product.

N-[2-(4-Amino-phenyl)-ethyl]-N-benzoyl-4-methyl benzenesulfonainide:

In a manner similar to procedure A, 0.245 g of nitro compound is combined with 0.015 g Pd/C in 10 mL EtOAc/MeOH and reduced to afford 0.247 g of the desired product

(4-{2-[Benzoyl-(toluene-4-sulfonyl)-amino]-ethyl}-phenyl sulfamic acid:

In a manner similar to procedure A, 0.247 g of the amine is treated with 0.299 g of sulfurtrioxide-pyridine in 5 mL pyridine. Workup and purification affords 0.173 g of the desired product as its ammonium salt. ¹H NMR (D₂O): δ 7.73 (d, J=8 Hz, 2H); 7.33-7.35 (m, 1H); 7.28 (d, J=8 Hz, 2H); 7.19 (t, J=15 Hz, 2H); 6.85 (d, J=8 Hz, 2H); 6.71 (d, J=8 Hz, 2H); 4.08 (t, J=12 Hz, 2H); 2.71 (t, J=12 Hz, 2H); 2.26 (s, 3H).

### Example 47

[4-(2-{tert-Butoxycarbonyl)[(3-fluoro-4-methylphenyl)sulfonyl]amino}ethyl)phenyl]sulfamic acid:

3-Fluoro-4-methyl-N-[2-(4-nitro-phenyl)-ethyl]-benzenesulfonamide:

In a manner similar to example 39, 0.500 g 4-nitrophenylethylamine hydrochloride is treated with 3.6 mL triethylamine, 0.515 g 3-fluoro-4-methyl benzenesulfonyl chloride in 20 mL THF. Work up and purification affords 0.434 g of the desired product.

tert-Butyl[(3-fluoro-4-methylphenyl)sulfonyl][2-(4-nitrophenyl)ethyl]carbamate:

In a manner similar to example 39, 0.434 g of sulfonamide is combined with 0.008 g DMAP, and 0.542 g di-tert-butyl dicarbonate in 25 mL methylene chloride. Workup affords 0.586 g of the desired product.

tert-Butyl[2-(4-amino-phenyl)-ethyl][3-fluoro-4-methyl-phenyl)-sulfonyl carbamate:

In a manner similar to procedure A, 0.533 g of nitro compound is combined with 0.032 g Pd/C in 10 mL EtOAc/MeOH and reduced to afford 0.497 g of the desired product.

[4-(2-{tert-Butoxycarbonyl)[(3-fluro-4-methylphenyl)sulfonyl]amino}ethyl)phenyl] sulfamic acid:

In a manner similar to procedure A, 0.129 g of the amine is treated with 0.151 g of sulfurtrioxide-pyridine in 5 mL pyridine. Workup and purification affords 0.044 g of the desired product as its ammonium salt. ¹H NMR (D₂O): δ 7.33-7.34 (m, 2H); 7.09 (d, J=8 Hz, 2H); 6.99 (d, J=7 Hz, 2H); 6.89 (s, 1H); 4.00 (t, J=13 Hz, 2H); 2.85 (t, J=13 Hz, 2H); 2.21 (s, 3H); 1.09 (s, 9H).

### Example 48

[4-(2-{(tert-Butoxycarbonyl) [(3-fluorophenyl)sulfonyl]amino)ethyl]phenyl}sulfamic acid:

3-Fluoro-N-[2-(4-nitro-phenyl)-ethyl]-benzenesulfonamide:

In a manner similar to example 39, 0.500 g 4-nitrophenylethylamine hydrochloride is treated with 3.6 mL triethylamine, and 0.480 g 3-fluorobenzenesulfonyl in 20 mL THF. Work up and purification affords 0.536 g of the desired product.

tert-Butyl[(3-fluorophenyl)sulfonyl][2-(4-nitrophenyl)ethyl]carbamate:

In a manner similar to example 39, 0.536 g of sulfonamide is combined with 0.010 g DMAP, and 0.433 g di-tert-butyl dicarbonate in 25 mL methylene chloride. Workup affords 0.586 g of the desired product.

tert-Butyl[2-(4-aminophenyl)ethyl][(3-fluorophenyl)sulfonyl]carbamate:

In a manner similar to procedure A, 0.586 g of nitro compound is combined with 0.035 g Pd/C in 10 mL EtOAc/MeOH and reduced to afford 0.153 g of the desired product after purification.

[4-(2-{(tert-Butoxycarbonyl)[(3-fluorophenyl)sulfonylcamino)ethyl]phenyl}sulfamic acid:

In a manner similar to procedure A, 0.153 g of the amine is treated with 0.186 g of sulfurtrioxide-pyridine in 5 mL pyridine. Workup and purification affords 0.016 g of the desired product as its ammonium salt. ¹H NMR (D₂O): δ 7.42-7.43 (m, 4H); 6.91 (s, 4H); 3.11 (t, J=13 Hz, 2H); 2.57 (t, J=7 Hz, 2H); 1.10 (s, 9H).

### Example 49

[4-(2-{(tert-Butoxycarbonyl)[(2-fluorophenyl)sulfonyl]amino)ethyl}phenyl]sulfamic acid:

2-Fluoro-N-[2-(4-nitro-phenyl)-ethyl]-benzenesulfonamide:

In a manner similar to example 39, 0.500 g 4-nitrophenylethylamine hydrochloride is treated with 3.6 mL triethylamine, and 0.480 g 2-fluorobenzenesulfonyl in 20 mL THF. Work up and purification affords 0.611 g of the desired product.

tert-Butyl[(2-fluorophenyl)sulfonyl][2-(4-nitrophenyl)ethyl]carbamate:

In a manner similar to example 393, 0.611 g of sulfonamide is combined with 0.012 g DMAP, and 0.493 g di-tert-butyl dicarbonate in 25 mL methylene chloride. Workup affords 0.705 g of the desired product.

tert-Butyl[2-(4-aminophenyl)ethyl][(2-fluorophenyl)sulfonyl] carbamate:

In a manner similar to procedure A, 0.705 g of nitro compound is combined with 0.042 g Pd/C in 10 mL EtOAc/MeOH and reduced to afford 0.638 g of the desired product.

[4-(2-{(tert-Butoxycarbonyl) [(2-fluorophenyl)sulfonyl]amino)ethyl}phenyl]sulfamic acid:

In a manner similar to procedure A, 0.639 g of the amine is treated with 0.772 g of sulfurtrioxide-pyridine in 8 mL pyridine. Workup and purification affords 0.071 g of the desired product as its ammonium salt. ¹H NMR (D₂O) δ 7.94 (t, J=15 Hz, 1H); 7.78-7.85 (m, 1H); 7.46-7.55 (m, 1H); 6.88-7.12 (m, 4H); 6.54 (d, J=8 Hz, 1H); 3.83-3.90 (m, 2H); 2.78-2.86 (m, 2H); 1.22 (s, 9H).

### Example 50

{4-[2-(Toluene-4-sulfonlyamino)-ethyl]-phenyl}-sulfamic acid:

N-[2-(4-Amino-phenyl)-ethyl]-4-methyl-benzenesulfonamide:

In a manner similar to procedure A, 0.407 g of 4-Methyl-N-[2-(4-nitro-phenyl)-ethyl]benzenesulfonamide compound is combined with 0.024 g Pd/C in 16 mL EtOAc/MeOH and reduced to afford 0.368 g of the desired product.

{4-[2-(Toluene-4-sulfonlyamino)-ethyl]-phenyl}-sulfamic acid:

In a manner similar to procedure A, 0.368 g of the amine is treated with 0.605 g of sulfurtrioxide-pyridine in 10 mL pyridine. Workup and purification affords 0.365 g of the desired product as its ammonium salt.

¹H NMR (D₂O) δ 7.49 (d, J=7.0 Hz, 2H); 7.26 (d, J=7.0 Hz, 2H); 6.90 (s, 4H); 3.01-3.06 (m, 2H); 2.51-2.55 (m, 2H); 2.30 (s, 3H).

### Example 51

[4-(2-Benzenesulfonylamino-ethyl)-phenyl]-sulfamic acid:

N-[2-(4-Nitro-phenyl)-ethyl]-benzenesulfonamide:

In a manner similar to example 39, 0.500 g 4-nitrophenylethylamine hydrochloride is treated with 3.6 mL triethylamine, and 0.480 g 3-fluorobenzenesulfonyl in 20 mL THF. Work up and purification affords 0.536 g of the desired product.

N-[2-(4-Amino-phenyl)-ethyl]-benzenesulfonamide:

In a manner similar to procedure A, 0.705 g of nitro compound is combined with 0.042 g Pd/C in 10 mL EtOAc/MeOH and reduced to afford 0.638 g of the desired product.

[4-(2-Benzenesulfonylamino-ethyl)-phenyl]-sulfamic acid:

In a manner similar to procedure A, 0.639 g of the amine is treated with 0.772 g of sulfurtrioxide-pyridine in 8 mL pyridine. Workup and purification affords 0.071 g of the desired product as its ammonium salt. ¹H NMR (D₂O) δ 7.54-7.64 (m, 3H); 7.43-7.48 (m, 2H); 6.91 (s, 4H); 3.033.07 (m, 2H); 2.52-2.57 (m, 2H).

### Example 52

[4-(2-Methanesulfonylamino-ethyl)-phenyl]-sulfamic acid:

N-[2-(4-Nitro-phenyl)-ethyl]-methanesulfonamide:

In a manner similar to example 39, 0.500 g 4-nitrophenylethylamine hydrochloride is treated with 3.6 mL triethylamine, and 0.480 g 3-fluorobenzenesulfonyl in 20 mL THF. Work up and purification affords 0.536 g of the desired product.

N-[2-(4-Amino-phenyl)-ethyl]-methanesulfonamide:

In a manner similar to procedure A, 0.705 g of nitro compound is combined with 0.042 g Pd/C in 10 mL EtOAc/MeOH and reduced to afford 0.638 g of the desired product

[4-(2-Methanesulfonylamino-ethyl)-phenyl]-sulfamic acid:

In a manner similar to procedure A, 0.639 g of the amine is treated with 0.772 g of sulfurtrioxide-pyridine in 8 mL pyridine. Workup and purification affords 0.071 g of the desired product as its ammonium salt. ¹H NMR (D₂O): δ 7.18 (d, J=8 Hz, 2H); 7.08 (d, J=8 Hz, 2H); 3.27 (t, J=6.8 Hz, 2H); 2.83 (s, 3H); 2.74 (t, J=6.8 Hz, 2H).

### Example 53

[4-(2-Methanesulfonylamino-ethyl)-phenyl]-sulfamic acid:

N-[2-(4-Nitro-phenyl)-ethyl]-C-phenyl-methanesulfonamide:

In a manner similar to example 39, 0.500 g 4-nitrophenylethylamine hydrochloride is treated with 3.6 mL triethylamine, and 0.480 g 3-fluorobenzenesulfonyl in 20 mL THF. Work up and purification affords 0.536 g of the desired product.

N-[2-(4-Amino-phenyl)-ethyl]-C-phenyl-methanesulfonamide:

In a manner similar to procedure A, 0.705 g of nitro compound is combined with 0.042 g Pd/C in 10 mL EtOAc/MeOH and reduced to afford 0.638 g of the desired product.

[4-(2-Methanesulfonylamino-ethyl)-phenyl]-sulfamic acid:

In a manner similar to procedure A, 0.639 g of the amine is treated with 0.772 g of sulfurtrioxide-pyridine in 8 mL pyridine. Workup and purification affords 0.071 g of the desired product as its ammonium salt. ¹H NMR (D₂O) δ 7.33-7.35 (m, 3H); 7.25-7.27 (m, 2H); 7.12 (d, J=8.4 Hz, 2H); 7.06 (d, J=8.4 Hz, 2H); 4.27 (s, 2H); 3.14 (t, J=6.8 Hz, 2H); 2.66 (t, J=6.8 Hz, 2H).

### Example 54

{4-[2-(4-Methoxy-benzenesulfonylamino)-ethyl]-phenyl}-sulfamic acid:

(S)-[4-Methoxy-N-[2-(4-Nitro-phenyl)-ethyl]-benzenesulfonamide:

In a manner similar to example 39, 0.500 g 4-nitrophenylethylamine hydrochloride is treated with 3.6 mL triethylamine, and 0.480 g 3-fluorobenzenesulfonyl in 20 mL THF. Work up and purification affords 0.536 g of the desired product.

(S)-N-[2-(4-Amino-phenyl)-ethyl]-4-methoxy-benzenesulfonamide:

In a manner similar to procedure A, 0.705 g of nitro compound is combined with 0.042 g Pd/C in 10 mL EtOAc/MeOH and reduced to afford 0.638 g of the desired product.

(S)-{4-[2-(4-Methoxy-benzenesulfonylamino)-ethyl]-phenyl}-sulfamic acid:

In a manner similar to procedure A, 0.639 g of the amine is treated with 0.772 g of sulfurtrioxide-pyridine in 8 mL pyridine. Workup and purification affords 0.071 g of the desired product as its ammonium salt. ¹H NMR (D₂O) δ 7.52 (d, J=7.3 Hz, 2H); 6.93 (d, J=7.5 Hz, 2H); 6.89 (s, 4H); 3.76 (s, 3H); 2.99 (t, J=6.7 Hz, 2H); 2.51 (t, J=6.7 Hz, 2H).

### Examples 55-59

The following chemical formula along with Table 4 shows the structure of compounds made according to the description of compounds made according to the description in Examples 55-59 described below:

TABLE 4

| | |
|---|---|
| | Formula (V) |
| 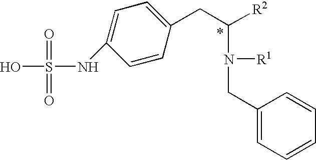 | |

| EXAMPLE | * | R¹ | R² |
|---|---|---|---|
| 55 | S | 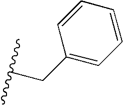 | 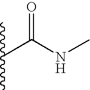 |
| 56 | S | 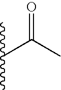 | 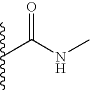 |
| 57 | S | 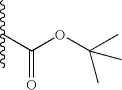 | 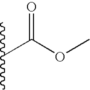 |
| 58 | S | 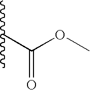 | 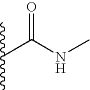 |
| 59 | S | 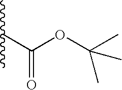 | 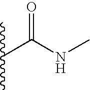 |

### Example 55

(S)-[4-(2-Dibenzylamino-2-methylcarbamoyl-ethyl)-phenyl]-sulfamic acid:

(S)-2-Dibenzylamino-N-methyl-3-(4-nitro-phenyl)-propionamide:

H-L-Phe(4-NO₂)—NMe (0.500 g) is dissolved in 10 mL water. To this solution is added 1.07 g potassium carbonate and 0.692 g benzyl bromide. The mixture is stirred for 48 hours and the reaction is partitioned between DCM and 1N HCl. The organics were dried over MgSO₄, filtered, evaporated, and purified by flash chromatography of afford 0.472 g of the desired product.

(S)-[4-(2-Dibenzylamino-2-methylcarbamoyl-ethyl)-phenyl]-sulfamic acid: In a similar manner to procedure A, 0.472 g of nitro compound and 0.10 g of palladium on carbon is reduced to give the desired product (tlc). This crude aniline compound is treated with 0.869 g of sulfur trioxide pyridine complex. Work up and purification yields 0.028 g of product as its ammonium salt. ¹H(D₂O): 6.93-6.69 (m, 14H), 3.37-3.03 (m, 5H), 2.68-2.46 (m, 2H), 2.27 (s, 3H)

### Example 56

(S)-{4-[2-(Acetyl-benzyl-amino)-2-methylcarbamoyl-ethyl]-phenyl}-sulfamic acid:

(S)-2-Benzylamino-N-methyl-3-(4-nitro-phenyl)-propionamide: H-L-Phe(4-NO₂)—NMe (0.300 g,) is dissolved in 4 mL MeOH and treated with 0.117 g triethylamine and 0.185 g benzaldehyde. The mixture is stirred for 1.5 hours then cooled in an ice bath. Sodium borohydride (0.88 g) is added and stirred for 1.5 hours at room temperature. The solvent is removed and the residue is partitioned between EtOAc and water. The organic layer is dried and purified by flash chromatography yield 0.300 g of product.

(S)-2-(Acetyl-benzyl-amino)-N-methyl-3-(4-nitro-phenyl)-propionamide: In a manner similar to example 2, 0.300 g of 2-Benzylamino-N-methyl-3-(4-nitro-phenyl)-propionamide is treated with 0.051 g triethylamine and 0.044 g of acetyl chloride. This affords 0.126 g of product.

(S)-{4-[2-(Acetyl-benzyl-amino)-2-methylcarbamoyl-ethyl]-phenyl}-sulfamic acid: In a similar manner to procedure A, 0.126 g of nitro compound and 50 mg of palladium on carbon is reduced to give the desired product (tlc). This crude aniline compound is treated with 0.169 g of sulfur trioxide pyridine complex. Work up and purification yields 0.034 g of product as its ammonium salt. ¹H(D₂O): 7.22-7.06 (m, 3H), 7.00-6.89 (m, 6H), 4.784.26 (m, 3H) 2.92 (t, 2H, J=8.3 Hz), 2.28 (s, 2H), 2.13 (s, 1H), 1.90 (s, 2H), 1.65 (s, 1H).

2-(Benzyl-tert-butoxycarbonyl-amino)-3-(4-sulfoamino-phenyl)-propionic acid methyl ester

### Example 57

(S)-2-(Benzyl-tert-butoxycarbonyl-amino)-3-(4-sulfoamino-phenyl)-propionic acid methyl ester:

(S)-2-Benzylamino-3(4-nitro-phenyl)-propionic acid methyl ester: In a manner similar to example 56, 0.525 g of H-L-Phe(4-NO₂)—OMe was treated with 0.204 g of triethylamine, 0.320 g of benzaldehyde, and 0.152 mg of sodium borohydride. This affords 0.241 g of product.

(S)-2-(Benzyl-tert-butoxycarbonyl-amino)-3-(4-nitro-phenyl)-propionic acid methyl ester: 2-Benzylamino-3-(4-nitro-phenyl)-propionic acid methyl ester (0.241 g) is dissolved in 10 mL DCM and treated with 0.78 g triethylamine and 0.502 g di-tert-butyl dicarbonate. The mixture is stirred for 72 hours. The mixture is partitioned between 25 mL DCM and 0.1N HCl. The aqueous layer is washed with DCM. The organic layers are combined, dried over MgSO₄, and purified by flash chromatography to yield 0.123 g of product.

(S)-2-(Benzyl-tert-butoxycarbonyl-amino)-3-(4-sulfoamino-phenyl)-propionic acid methyl ester: In a similar manner to procedure A, 0.123 g of nitro compound and 0.050 g of palladium on carbon is reduced to give the desired product (tlc). This crude aniline compound is treated with 0.142 g of sulfur trioxide pyridine complex. Work up and purification yields 0.015 g of product as its ammonium salt. ¹H(D₂O) 7.10 (s, 3H), 6.98-6.86 (m, 6H), 4.234.01 (m, 2H), 3.77 and 3.72 (d, 1H), 3.55-3.41 (m, 3H), 3.04-2.86 (m, 2H), 1.25 and 1.16 (d, 9H)

### Example 58

(S)-Benzyl-[1-methylcarbamoyl-2-(4-sulfoamino-phenyl)-ethyl]-carbamic acid methyl ester:

(S)-Benzyl-[1-methylcarbamoyl-2-(4-nitro-phenyl)-ethyl]-carbamic acid methyl ester

In a manner similar to example 2, 0.200 g of 2-Benzylamino-N-methyl-3-(4-nitro-phenyl)-propionamide is treated with 0.071 g triethylamine, and 0.066 g methyl chloroformate. This affords 0.060 g of product.

(S)-Benzyl-[1-methylcarbamoyl-2-(4-sulfoamino-phenyl)-ethyl]-carbamic acid methyl ester:

In a similar manner to procedure A, 0.060 g of nitro compound is combined with 0.025 g of palladium on carbon and reduced to give the desired product. This crude aniline compound is treated with 0.077 g of sulfur trioxide pyridine complex. Work up and purification yields 0.012 g of product as its ammonium salt. ¹H(D₂O): 7.26-7.11 (m, 3H), 7.03-6.88 (m, 5H), 4.51 (m, 1H), 4.35 (s, 2H), 3.49 (s, 3H), 2.95 (s, 2H), 2.36 (s, 3H)

### Example 59

(S)-Benzyl-[1-methylcarbamoyl-2-(4-sulfoamino-phenyl)-ethyl]-carbamic acid tert-butyl ester:

(S)-Benzyl-[1-methylcarbamoyl-2-(4-nitro-phenyl)-ethyl]-carbantic acid tert-butyl ester: 2(Benzyl-tert-butoxycarbonyl-amino)-3-(4-nitro-phenyl)-propionic acid methyl ester (0.245 g) is dissolved in 10 mL 1:1 EtOH:EtOAC and is treated with 0.124 g lithium hydroxide monohydrate. The mixture is stirred for 3 hr. at room temperature. The solvents are evaporated and the residue is partitioned between DCM and 0.1N HCl. The combined organics are dried and the afford the crude acid. The acid is combined with 5 mL THF and in a manner similar to Example 1, is treated with 0.050 g NMM, 0.061 g isobutylchloroformate, and 0.450 mL of methylamine solution. This affords 0.161 g of the desired product.

(S)-Benzyl-[1-methylcarbamoyl-2-(4-sulfoamino-phenyl)-ethyl]-carbamic acid tert-butyl ester: In a similar manner to procedure A, 0.161 g of nitro compound is combined with 0.025 g of palladium on carbon and reduced to give the desired product. This crude aniline compound is treated with 0.186 g of sulfur trioxide pyridine complex. Work up and purification yields 0.058 g of product as its ammonium salt. ¹H(D₂O): 7.26-7.14 (m, 3H), 7.07-7.00 (m, 6H), 4.34-4.01 (m, 3H), 2.99 and 2.96 (d, 2H, J=8.9), 2.45 (s, 3H), 1.28 (s, 9H)

### Examples 60-66

The following chemical formula along with Table 5 shows the structure of compounds made according to the description in Examples 60-66 described below.

TABLE 5

| | |
|---|---|
| | Formula (VI) |
| 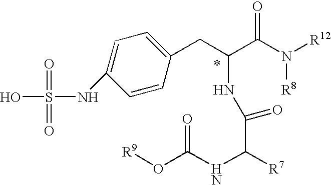 | |

| EXAMPLE | * | R⁷ | R⁸ | R⁹ | R¹² |
|---|---|---|---|---|---|
| 60 | S | 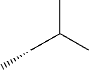 | H | 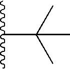 | —CH₃ |
| 61 | S | 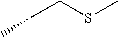 | H | 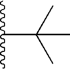 | —CH₃ |
| 62 | S | 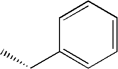 | H | 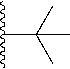 | —CH₃ |
| 63 | S | 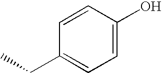 | H | 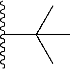 | —CH₃ |
| 64 | S | 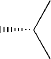 | H | 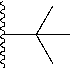 | —CH₃ |
| 65 | S | 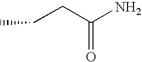 | H | 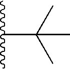 | —CH₃ |
| 66 | S | 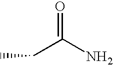 | H | 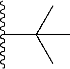 | —CH₃ |

### Example 60

N-[(1,1-dimethylethoxy)carbonyl]-L-leucinyl-N-methyl-L-4-sulfoamino-phenylalaninamide:

N-[(1,1-dimethylethoxy)carbonyl]-L-leucinyl-N-methyl-L-4-nitro-phenylalaninamide: H-L-Phe(4-NO₂)—NMe (200 mg) is dissolved in 1 mL DMF and treated with 0.209 g diisopropylethylamine, 0.130 g HOBt.H₂O, 0.211 g L-Boc-Leu, and 0.162 g EDCI. After 18 hours, the mixture is partitioned between water and EtOAc. The combined organics are dried and purified by flash chromatography to afford 0.193 g of product.

N-[(1,1-dimethylethoxy)carbonyl]-L-leucinyl-N-methyl-L-4-sulfoamino-phenylalaninamide:: In a similar manner to procedure A, 0.193 g of nitro compound is combined 0.025 g of palladium on carbon and reduced to give the desired product(tlc). This crude aniline compound is treated with 0.211 g of sulfur trioxide pyridine complex. Work up and purification yields 0.048 g of product as its ammonium salt. ¹H(D₂O): 7.19-6.99 (m, 4H), 4.43 (t, 1H, J=8.5), 3.86 (t, 1H, J=6.3 Hz), 3.04-2.86 (m, 2H), 2.58 (s, 1H), 1.48-1.40 (m, 3H), 1.33 (s, 9H), 0.81-0.70 (m, 6H)

### Example 61

N-[(1,1-dimethylethoxy)carbonyl]-L-methionyl-N-methyl-L-4-sulfoamino-phenylalaninamide:

N-[(1,1-dimethylethoxy)carbonyl]-L-methionyl-N-methyl-L-4-nitro-phenylalaninamide: In a manner similar to example 60, 0.200 g H-L-Phe(4-NO₂)—NMe is treated with 0.209 g diisopropylethylamine, 0.211 g L-Boc-Met, 0.130 g HOBt.H₂O, and 0.162 g EDCI. This affords 0.122 g of product.

N-[(1,1-dimethylethoxy)carbonyl]-L-methionyl-N-methyl-L-4-sulfoamino-phenylalaninamide: In a similar manner to procedure A, 0.122 g of nitro compound and 0.025 g of palladium on carbon is reduced to give the desired product(tlc). This crude aniline compound is treated with 0.128 g of sulfur trioxide pyridine complex. Work up and purification yields 0.013 g of product as its ammonium salt. ¹H(D₂O): 7.34-6.94 (m, 4H), 4.39 (t, 1H, J=8.8), 3.96 (t, 1H, J=8.3 Hz), 2.98-2.76 (m, 2H), 2.53 (s, 3H), 2.31 (m, 2H), 1.93 (s, 3H), 1.70-1.55 (m, 2H), 1.28 (s, 9H).

### Example 62

N-[(1,1-dimethylethoxy)carbonyl]-L-phenylalanyl-N-methyl-L-4-sulfoamino-phenylalaninamide:

N-[(1,1-dimethylethoxy)carbonyl]-L-phenylalanyl-N-methyl-L-4-nitro-phenylalaninamide: In a manner similar to example 60, 0.200 g H-L-Phe(4-NO₂)—NMe is treated with 0.209 g diisopropylethylamine, 0.225 g L-Boc-Phe, 0.130 g HOBt.H₂O, and 0.162 g EDCI. This affords 0.246 g of product.

N-[(1,1-dimethylethoxy)carbonyl]-L-phenylalanyl-N-methyl-L-4-sulfoamino-phenylalaninamide: In a similar manner to procedure A, 0.246 g of nitro compound is combined with 0.050 g of palladium on carbon and reduced to give the desired product. This crude aniline compound is treated with 0.250 g of sulfur trioxide pyridine complex. Work up and purification yields 0.043 g of product as its ammonium salt. ¹H(D₂O) 7.26-7.17 (m, 3H), 7.06-6.99 (m, 6H), 4.31 (t, 1H, J=9.0), 4.12 (t, 1H, J=8.3 Hz), 2.84-2.72 (m, 4H), 2.49 (s, 3H), 1.22 (s, 9H)

### Example 63

N-[(1,1-dimethylethoxy)carbonyl]-L-tyrosinyl-N-methyl-L-4-sulfoamino-phenylalaninamide:

N-[(1,1-dimethylethoxy)carbonyl]-L-tyrosinyl-N-methyl-L-4-nitro-phenylalaninamide: In a manner similar to example 60, 0.200 g H-L-Phe(4-NO₂)—NMe is treated with 0.209 g diisopropylethylamine, 0.238 g L-Boc-Tyr, 0.130 mg HOBt.H₂O, and 0.162 g EDCI. This affords 0.300 g of product.

N-[(1,1-dimethylethoxy)carbonyl]-L-tyrosinyl-N-methyl-L-4-sulfoamino-phenylalaninamide: In a similar manner to procedure A, 0.300 g of nitro compound is combined with 0.050 g of palladium on carbon and reduced to give the desired product(tlc). This crude aniline compound is treated with 0.295 g of sulfur trioxide pyridine complex. Work up and purification yields 0.062 g of product as its ammonium salt. ¹H(D₂O): 7.00-6.87 (m, 6H), 6.69 and 6.66 (d, 2H, J=9.3 Hz), 4.28 (t, 1H, J=8.0), 4.05 (t, 1H, J=8.7 Hz), 2.81-2.65 (m, 4H), 2.47 (s, 3H), 1.21 (s, 9H).

### Example 64

N-[(1,1-dimethylethoxy)carbonyl]-L-valinyl-N-methyl-L-4-sulfoamino-phenylalaninamide:

N-[(1,1-dimethylethoxy)carbonyl]-L-valinyl-N-methyl-L-4-nitro-phenylalaninamide: In a manner similar to Example 60, 0.200 g H-L-Phe(4-NO₂)—NMe is treated with 0.209 g diisopropylethylamine, 0.184 g L-Boc-Val, 0.130 g HOBt.H₂O, and 0.162 g EDCI. This affords 0.219 g of product.

N-[(1,1-dimethylethoxy)carbonyl]-L-valinyl-N-methyl-L-4-sulfoamino-phenylalaninamide:: In a similar manner to procedure A, 0.219 g of nitro compound is combined with 0.050 g of palladium on carbon and reduced to give the desired product(tlc). This crude aniline compound is treated with 0.247 g of sulfur trioxide pyridine complex. Work up and purification yields 0.041 g of product as its ammonium salt. ¹H(D₂O): 7.08-6.99 (q, 4H, J=11 Hz), 4.40 (t, 1H, J=8.3), 3.64 (t, 1H, J=6.7 Hz), 2.99-2.83 (m, 2H), 2.52 (s, 3H), 1.81 (m, 1H), 1.25 (s, 9H), 0.68 (s, 6H).

### Example 65

N-[(1,1-dimethylethoxy)carbonyl]-L-glutaminyl-N-methyl-L-4-sulfoaminophenylalaninamide:

N-[(1,1-dimethylethoxy)carbonyl]-L-glutaminyl-N-methyl-L-4-nitro-phenylalaninamide:: H-L-Phe(4-NO₂)—NMe 0.300 g is dissolved in anhydrous 4 mL MeOH and treated with 0.315 g diisopropylethylamine and 0.404 g L-Boc-Gln-ONp. The mixture is heated to 60° C. and stirred for 18 hours. The mixture is cooled and the solution is partitioned between EtOAc and H₂O. The organic layer is washed with H₂O and dried. The crude oil is recrystallized from 1:1 EtOAc:hexanes to afford 0.198 g of product.

N-[(1,1-dimethylethoxy)carbonyl]-L-glutaminyl-N-methyl-L-4-sulfoamino-phenylalaninamide: In a similar manner to procedure A, 0.198 g of nitro compound is combined with 0.050 g of palladium on carbon and reduced to give the desired product(tlc). This crude aniline compound is treated with 0.209 g of sulfur trioxide pyridine complex. Work up and purification yields 0.082 g of product as its ammonium salt. ¹H(D₂O): 7.08-7.00 (q, 4H, J=9.7 Hz), 4.40 (t, 1H, J=8.0), 3.83 (t, 1H, J=9.0 Hz), 2.95-2.85 (m, 2H), 2.54 (s, 3H), 2.07 (t, 2H, J=7.3 Hz), 1.73-1.66 (m, 2H), 1.30 (s, 9H).

### Example 66

N-[(1,1-dimethylethoxy)carbonyl]-L-asparaginyl-N-methyl-L-4-sulfoamino-phenylalaninamide:

N-[(1,1-dimethylethoxy)carbonyl]-L-asparginyl-N-methyl-L-4-nitro-phenylalaninamide: In a manner similar to example 65, 0.300 g of H-L-Phe(4-NO₂)—NMe is treated with 0.315 g of diisopropylethylamine, and 0.388 g of L-Boc-Asn-ONp. This affords 0.176 g of product.

N-[(1,1-dimethylethoxy)carbonyl]-L-asparaginyl-N-methyl-L-4-sulfoamino-phenylalaninamide: In a similar manner to procedure A, 0.176 g of nitro compound is combined with 0.050 g of palladium on carbon and reduced to give the desired product(tlc). This crude aniline compound is treated with 0.192 g of sulfur trioxide pyridine complex. Work up and purification yields 0.047 g of product as its ammonium salt. ¹H(D₂O): 7.07-7.00 (q, 4H, J=10.0 Hz), 4.38 (t, 1H, J=8.0), 4.25 (t, 1H, J=10.0 Hz), 2.97-2.85 (m, 2H), 2.54 (s, 3H), 2.48-2.30 (m, 2H), 1.29 (s, 9H)

### Examples 67-68

The following chemical formula along with Table 6 shows the structure of compounds made according to the description in Examples 67-68 described below:

TABLE 6

| | |
|---|---|
| | Formula (VII) |
| 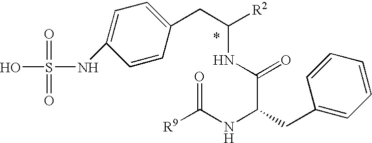 | |

| EX- | | | |
|---|---|---|---|
| AMPLE | * | R² | R⁹ |
| 67 | S | 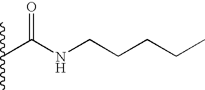 | 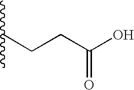 |
| 68 | S | 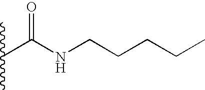 | 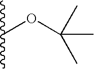 |

### Example 67

N-{1-[1-Pentylcarbamoyl-2-(4-sulfoamino-phenyl)-ethylcarbamoyl]-2-phenyl-ethyl}-succinamic acid:

[(1S)-1-[(4-nitrophenyl)methyl]-2-oxo-2-(pentylamino)ethyl]-carbamic acid 1,1-dimethylethyl ester: In 40 mL of dichloromethane is added 3.103 g of L-Boc 4-NO₂Phe. EDCI (1.917 g) is added at ice-bath temperature. Pentylamine (1.16 mL) is added and the reaction is warmed to ambient temperature. After 18 h, the reaction is diluted with dichloromethane and washed twice with 1N HCl, dried over sodium sulfate, and purified by flash chromatography to yield 1.866 g of product.

(2S)-2-Amino-3-(4-nitrophenyl)-N-pentyl-propionaniide hydrochloride: [(1S)-1-[(4-nitrophenyl)methyl]-2-oxo-2-(pentylamino)ethyl]-carbamic acid 1,1-dimethylethyl ester (1.858 g) is treated with 18 mL of 4M HCl in dioxane. After 2 hours the reaction is concentrated and used without purification in the next reaction.

N-[(1,1-dimethylethoxy)carbonyl]-L-phenylalanyl-4-nitro-N-pentyl-L-Phenylalaninamide: (2S)-2-Amino-3-(4-nitrophenyl)-N-pentyl-propionamide hydrochloride is combined with 25 mL dichloromethane and 2.1 mL triethylamine. HOBt (0.662 g) and 1.300 g of L-BocPhe is added. EDCI (0.939 g) is added to the mixture at ice-bath temperature. The reaction is then allowed to warm to ambient temperature. After 18 hours the reaction is diluted with EtOAc, washed twice with 1N HCl, and dried over sodium sulfate. Purification by flash chromatography gives 1.732 g of product.

L-phenylalanyl-4-nitro-N-pentyl-L-Phenylalaninamide Hydrochloride: N-[(1,1 dimethylethoxy)carbonyl]-L-phenylalanyl-4-nitro-N-pentyl-L-Phenylalaninamide is (0.400 g) is combined with 8 mL 4M HCl in dioxane. After 1.5 hours the reaction is concentrated and used without purification in the next reaction.

N-(4-hydroxy-1,4-dioxobutyl)-L-phenylalanyl-4-nitro-N-pentyl-L-Phenylalaninamide: L-phenylalanyl-4-nitro-N-pentyl-L-Phenylalaninamide Hydrochloride is combined with 0.22 mL of triethylamine. The solution is cooled in an ice-bath and 0.077 g of succinic anhydride. The reaction is stirred at ambient temperature for 1.5 hours and concentrated. The product is used without purification in the next reaction.

N-(4-hydroxy-1,4-dioxobutyl)-L-phenylatanyl-4-amino-N-pentyl-L-Phenylalaninamide: In a manner similar to procedure A, 0.240 g of N-(4-hydroxy-1,4-dioxobutyl)-L-phenylalanyl-4-nitro-N-pentyl-L-Phenylalaninamide (0.240 g) is combined with 20 mL 1:1 EtOH/TBF and 0.200 g 10% Pd/C and reduced to afford 0.215 g of product.

N-{1-[1-Pentylcarbamoyl-2-(4-sulfoamino-phenyl)-ethylcarbamoyl]-2-phenyl-ethyl}-succinamic acid: In a manner similar to procedure A, 0.215 g of amine is treated with 0.191 g of SO₃ pyridine complex. Purification yields 0.096 g of product. ¹H NMR (D₂O) 0.72 (3H, t, J=7.1 Hz), 0.94-1.22 (6H, m), 2.81-2.96 (10H, m), 4.26 (1H, t, J=7.7 Hz), 4.37 (1H, t, J=7.7 Hz), 6.997.06 (5H, m), 7.16-7.23 (4H, m).

### Example 68

N-{1-L-[1-Pentylcarbamoyl-2-(4-sulfoamino-phenyl)-ethylcarbamoyl]-2-L-phenyl-ethyl}-carbamic acid tert-butyl ester:

N-[(1,1-dimethylethoxy)carbonyl]-L-phenylalanyl-4-aniino-N-pentyl-L-Phenylalaninamide:: In a manner similar to procedure A, 0.280 g of N-[(1,1-dimethylethoxy)carbonyl]-L-phenylalanyl4-nitro-N-pentyl-L-Phenylalaninamide is combined with 10 mL 1:1 MeOH/THF and 0.165 g Pd/C and placed under hydrogen atmosphere. After workup, the crude amine is used without purification in the next reaction.

N-{1-L-[1-Pentylcarbamoyl-2-(4-sulfoamino-phenyl)-ethylcarbamoyl]-2-L-phenyl-ethyl}-carbamic acid tert-butyl ester: In a manner similar to procedure A, 0.253 g of amine is treated with 0.470 g of sulfurtrioxide-pyridine in 5 mL pyridine. After workup and purification 0.150 g of product is isolated as its ammonium salt. ¹H NMR (D₂O) 7.26-7.20 (9H, m), 4.32 (1H, t, J=7.3 Hz), 4.16 (1H, t, J=7.3 Hz), 2.96-2.81 (6H, m), 1.25 (9H, s), 1.21-1.09 (2H, m), 0.98 (2H, m), 0.74 (3H, t, J=7.2 Hz).

### Examples 69-73

### Example 69

(S)-2-tert-Butoxycarbonylamino-3-(4-sulfoamino-phenyl)-propionic acid methyl ester:

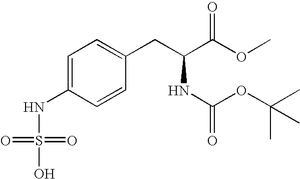

Boc-L-Phe(4-NO₂)—OMe: Boc-L-Phe(4-NO₂)—OH (0.400 g) is dissolved in 20 mL methanol. (Trimethylsilyl)diazomethane (6.4 mL, 2.0 M in hexanes) is added dropwise at 0° C. until the solution remains yellow at which point the mixture is quenched with AcOH until the solution remains colorless. The mixture is then concentrated to afford 0.423 g of the desired product.

(S)-2-tert-Butoxycarbonylamino-3-(4-sulfoamino-phenyl)-propionic acid methyl ester: In a manner similar to procedure A, 0.423 g of nitro compound and 100 mg of palladium on carbon is reduced to give the desired product (tlc). This crude aniline compound is treated with 0.623 g of sulfurtrioxide-pyridine complex. Work up and purification yields 0.134 g of product as its ammonium salt. ¹H(D₂O): 7.15-7.09 (q, 4H, J=10.1 Hz), 4.34 (t, 1H, J=6.0 Hz), 3.68 (s, 3H), 3.08-2.83 (m, 2H), 1.32 (s, 9H).

### Example 70

[2-(4-Sulfoamino-phenyl)-ethyl]-carbamic acid tert-butyl ester:

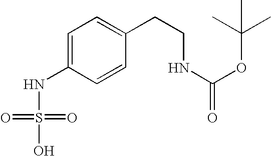

[2-(4-Nitro-phenyl)-ethyl]-carbamic acid tert-butyl ester: 4-nitrophenylethylamine hydrochloride (0.500 g) is dissolved in 5 mL ethanol with 0.723 mL triethylamine and BOC-ON (0.669 g). The mixture is stirred for 10 min at room temperature. The mixture is concentrated and partitioned between DCM (25 mL) and 0.1N HCl. The organic layer is washed with brine, dried over MgSO₄, filtered and evaporated to give crude material which is then chromatographed to provide 0.352 g of the desired product.

[2-(4-Sulfoamino-phenyl)-ethyl]-carbamic acid tert-butyl ester: In a similar manner to procedure A, 0.352 g of nitro compound and 100 mg of palladium on carbon is reduced to give the desired product (tlc). This crude aniline compound is treated with 0.631 g of sulfurtrioxide-pyridine complex. Work up and purification yields 0.160 g of product as its ammonium salt.

¹H(D₂O): 7.08-6.99 (q, 4H, J=7.2 Hz), 3.16 (t, 2H, J=7.4 Hz), 2.59 (t, 3H, J=7.4 Hz), 1.23 (s, 9H).

### Example 71

[4-(2-Diphenylacetylamino-ethyl)-phenyl]-sulfamic acid:

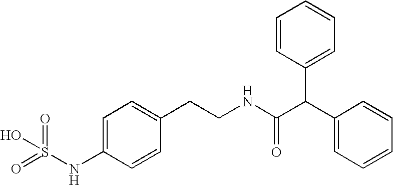

1N-[2-(4-Nitro-phenyl)-ethyl]-2,2-diphenyl-acetamide: In 10 mL of dichloromethane 0.300 g of 4-NO₂ Phenylethylamine hydrochloride is combined with 0.49 mL of triethylamine. To this solution is added 0.404 g of diphenylacetyl chloride. After 18 h, the reaction is washed with 1N HCl twice and once with brine. The crude product was purified by flash chromatography to afford 0.285 g of product.

1N-[2-(4-Amino-phenyl)-ethyl]-2,2-diphenyl-acetamide: In a manner similar to procedure A, 0.285 g of 1N-[2-(4-Nitro-phenyl)-ethyl]-2,2-diphenyl-acetamide is treated with 0.200 g of 10% Pd/C in 6 mL ethanol and 20 mL THF and reduced to afford the desired product.

[4-(2-Diphenylacetylamino-ethyl)-phenyl]-sulfamic acid: In a dry flask, 1N-[2-(4-Amino-phenyl)-ethyl]-2,2-diphenyl-acetamide is dissolved in methylene chloride. The solution is treated with 0.064 mL pyridine and 0.12 mL of ClSO₃TMS. After 5 hours the reaction was concentrated and treated with 20 mL water and 2 mL ammonium hydroxide. The aqueous layer was washed twice with ether and then concentrated. Purification yields 0.083 g of product. ¹H NMR (D₂O) 2.62 (2H, t, J=6.6 Hz), 3.37 (2H, t, J=6.6 Hz), 4.85 (1H, s), 6.95-7.01 (8H, m), 7.20-7.25 (6H, m).

### Example 72

(S)-[4-(3-Acetyl-1,2,2-trimethyl-5-oxo-imidazolidin-4-ylmethyl)-phenyl]-sulfamic acid:

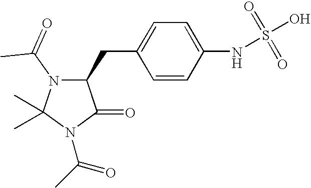

(S)-2,2,3-Trimethyl-5-(4-nitro-benzyl)-imidazolidin-4-one: H-L-Phe(4-NO₂)—NMe (0.500 g) is dissolved in 2 mL methanol and treated with 0.71 mL acetone and 0.037 g pTSA and heated to reflux for 76 hours. The mixture is concentrated and purified by flash chromatography to provide 0.339 g of product.

(S)-3-Acetyl-2,2,3-trimethyl-5-(4-nitro-benzyl)-imidazolidin-4-one: In a manner similar to example 2, 0.339 g of 2,2,3-Trimethyl-5-(4-nitro-benzyl)-imidazolidin-4-one is treated with 0.18 mL triethylamine, and 0.111 g acetyl chloride. This affords 0.344 g of product.

(S)-[4-(3-Acetyl-1,2,2-trimethyl-5-oxo-imidazolidin-4-ylmethyl)-phenyl]-sulfamic acid: In a similar manner to procedure A, 0.344 g of nitro compound is combined with 0.050 g of palladium on carbon and reduced to give the desired product (tlc). This crude aniline compound is treated with 0.538 g of sulfur trioxide pyridine complex. Work up and purification yields 0.049 g of product as its ammonium salt. ¹H(D₂O): 6.97-6.76 (m, 4H), 4.50 (m, 1H), 3.09-3.03 (m, 2H) 2.55 (s, 3H), 2.13 (s, 3H) 1.37 (s, 3H), 0.45 (s, 3H).

### Example 73

N-[(1,1-dimethylethoxy)carbonyl]-L-prolinyl-N-methyl-L-4-sulfoamino-phenylalaninamide:

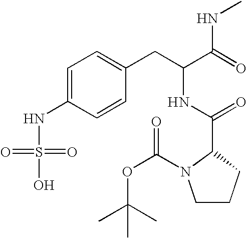

N-[(1,1-dimethylethoxy)carbonyl]-L-prolinyl-N-methyl-L-4-nitro-phenylalaninamide: In a manner similar to example 60, 0.200 gH-L-Phe(4-NO₂)—NMe is treated with 0.209 g diisopropylethylamine, 0.182 g L-Boc-Pro, 0.130 g HOBt.H₂O, and 0.162 g EDCI. This affords 0.220 g of product.

N-[(1,1-dimethylethoxy)carbonyl]-L-prolinyl-N-methyl-L-4-sulfoamino-phenylalaninamide: In a similar manner to procedure A, 0.220 g of nitro compound is combined with 0.050 g of palladium on carbon and reduced to give the desired product(tlc). This crude aniline compound is treated with 0.250 g of sulfur trioxide pyridine complex. Work up and purification yields 0.066 g of product as its ammonium salt. ¹H(D₂O): 7.12-6.92 (m, 4H), 4.33 (t, 1H, J=8.9), 4.06-4.01 (m, 1H), 3.24 (t, 2H, J=7.5 Hz), 2.87-2.79 (m, 2H), 2.50 (s, 3H), 2.02 (m, 1H), 1.68-1.50 (m, 3H), 1.15 (s, 9H).

### Example A

A tablet composition for oral administration, according to the present invention, is made comprising:

| Component | Amount |
|---|---|
| Example 1 compound | 150 mg |
| Lactose | 120 mg |
| Maize Starch | 70 mg |
| Talc | 4 mg |
| Magnesium Stearate | 1 mg |

### Example B

A capsule containing 200 mg of active for oral administration, according to the present invention, is made comprising:

| Component | Amount (% w/w) |
|---|---|
| Example 2 compound | 15% |
| Hydrous Lactose | 43% |
| Microcrystalline Cellulose | 33% |
| Crosspovidone | 3.3% |
| Magnesium Stearate | 5.7% |

Other subject compounds are used with substantially similar results.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited are, in relevant part, incorporated herein by reference; the citation of any document is not to be construed as an admission that it is prior art with respect to the present invention

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope

What is claimed is:

1. A compound according to formula (I): 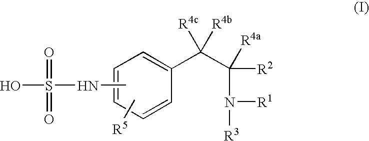
wherein:
A) R¹ is -L¹-[C(R^{6a}R^{6b})]ₘR⁷, wherein:
a) L¹ is selected from the group consisting of covalent bond, —O—, —S—, —N—, —CO₂—, —CO—, —OCO₂—, —SO—, —SO₂—, —CSN(R⁸)—, —CON(R⁸)O—, —CON(R⁸)—, —OCON(R⁸)—; wherein R⁸ is hydrogen or substituted or unsubstituted C₁-C₅ alkyl;
b) R^{6a} and R^{6b} are each independently selected from the group consisting of hydrogen, —OR⁹, —N(R⁹)₂, —CO₂R⁹, —CON(R⁹)₂, —NHCOR⁹, —NHCO₂R⁹, ═NR⁹, —R⁹, and mixtures thereof; wherein each R⁹ is independently selected from the group consisting of hydrogen, substituted or unsubstituted C₁-C₅ alkyl, and substituted or unsubstituted aryl or alkylenearyl; or two R⁹ units can be taken together to form a substituted or unsubstituted carbocyclic or heterocyclic ring comprising from 3 to 7 atoms;
c) m is an index selected from 0 to 5;
d) R⁷ is selected from the group consisting of nil, hydrogen, substituted or unsubstituted C₁-C₁₀ alkyl, substituted or unsubstituted C₁-C₁₀ heteroalkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
e) R⁷ and a R⁹ can be taken together to form a substituted or unsubstituted carbocyclic or heterocyclic ring comprising from 3 to 7 atoms;
B) R² is —(CH₂)ⱼ-L²-[C(R^{11a}R^{11b})]_{g}R¹², wherein:
a) j is an index selected from 0 to 5;
b) L² is selected from the group consisting of covalent bond, —O—, —S—, —N—, —CO₂—, —CO—, —OCO₂—, —SO—, —SO₂—, —CSN(R¹⁰)—, —CON(R¹⁰)—, —CON(R¹⁰)O—, —OCON(R¹⁰)—; wherein R¹⁰ is hydrogen or substituted or unsubstituted C₁-C₅ alkyl;
c) R^{11a} and R^{11b} are each independently selected from the group consisting of hydrogen, —OR¹³, —N(R¹³)₂, —CO₂R¹³, —CON(R¹³)₂, —NHCOR¹³, —NHCO₂R¹³, ═NR¹³, —R¹³, and mixtures thereof; wherein each R¹³ is independently selected from the group consisting of hydrogen, substituted or unsubstituted C₁-C₅ alkyl, and substituted or unsubstituted aryl or alkylenearyl; or two R¹³ units can be taken together to form a substituted or unsubstituted carbocyclic or heterocyclic ring comprising from 3 to 7 atoms;
d) g is an index selected from 0 to 5;
e) R¹² is selected from the group consisting of nil, hydrogen, substituted or unsubstituted C₁-C₁₀ alkyl, substituted or unsubstituted hydrocarbyl, substituted or unsubstituted heterocyclyl, substituted or unsubstituted aryl or alkylenearyl, substituted or unsubstituted heteroaryl or alkyleneheteroaryl; or
f) R¹² and a R¹³ can be taken together to form a substituted or unsubstituted carbocyclic or heterocyclic ring comprising from 3 to 7 atoms;
C) R³ is —(CH₂)ₙ-L³-R¹⁶, wherein:
a) n is an index selected from 0 to 5;
b) L³ is selected from covalent bond, —O—, —S—, —N—, —CO₂—, —CO—, —OCO₂—, —SO—, —SO₂—, —CSNH—, —CONH—, —OCONH—;
c) R¹⁶ is selected from the group consisting of hydrogen, substituted or unsubstituted C₁-C₁₀ alkyl, substituted or unsubstituted C₁-C₁₀ heteroalkyl substituted or unsubstituted aryl or alkylenearyl, substituted or unsubstituted heterocyclyl, substituted or unsubstituted heteroaryl or alkyleneheteroaryl;
D) R^{4a}, R^{4b}, R^{4c} and R⁵ are each independently selected from hydrogen or substituted unit; or
E) R² and R^{4a}, R^{4a} and R^{4b}, R¹ and R², or R¹ and R³ can be taken together to form a substituted or unsubstituted carbocyclic or heterocyclic ring comprising from 3 to 7 atoms.

2. The compound of claim 1 having the formula (II):

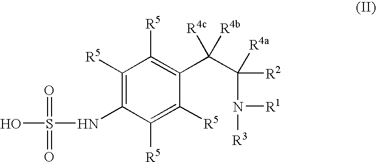

3. The compound of claim 2, wherein:
a) j is 0;
b) L is —CON(R¹⁰)—; and
c) g is 0.

4. The compound of claim 3, wherein R³ is hydrogen.

5. The compound of claim 4, wherein L¹ is selected from the group consisting of —CO₂—, —CO—, —SO₂—, and —CON(R⁸)—

6. The compound of claim 2, wherein:
a) j is 0;
b) L² is —CON(R¹⁰)—; and
c) g is 1.

7. The compound of claim 6, wherein R³ is hydrogen.

8. The compound of claim 7, wherein at least R^{11a} or R^{11b} is —CONH₂.

9. The compound of claim 2, wherein R² is hydrogen.

10. The compound of claim 9, wherein L¹ is —SO₂—.

11. The compound of claim 10, wherein L³ is selected from covalent bond, —CO—, and —CO₂.

12. The compound of claim 10, wherein R³ is hydrogen.

13. The compound of claim 10, wherein:
a) m is an index selected from 1 and 2; and
b) R⁷ is substituted or unsubstituted phenyl.

14. The compound of claim 2, wherein R³ is benzyl.

15. The compound of claim 14, wherein:
a) j is 0; and
b) L² is selected from —CO₂— and —CON(R₈)—.

16. The compound of claim 2, wherein:
a) j is 0;
b) L¹ is —CO—;
c) m is 1;
d) R^{6a} or R^{6b} is at least —NHCO₂R⁹; and
e) L² is —CON(R⁸)—;

17. The compound of claim 2, wherein:
a) j is 0;
b) L¹ is —CO—;
c) m is 1;
d) R^{6a} or R^{6b} is at least —NHCO₂R⁹; and
e) R⁷ is benzyl.

18. The compound of claim 1, wherein the compound is selected from the group consisting of: (R)-[1-Methylcarbamoyl-2-(4-sulfoamino-phenyl)-ethyl]-carbamic acid tert-butyl ester; (R)-[1-Methylcarbamoyl-2-(4-sulfoamino-phenyl)-ethyl]-carbamic acid benzyl ester; (S)-[1-Methylcarbamoyl-2-(4-sulfoamino-phenyl)-ethyl]-carbamic acid benzyl ester; (S)-[1-Methylcarbamoyl-2-(4-sulfoamino-phenyl)-ethyl]-carbamic acid tert-butyl ester; (R)-[1-Pentylcarbamoyl-2-(4-sulfoamino-phenyl)-ethyl]-carbamic acid tert-butyl ester; (R)-[1-Benzylcarbamoyl-2-(4-sulfoamino-phenyl)-ethyl]-carbamic acid tert-butyl ester; (S)-[1-Benzylcarbamoyl-2-(4-sulfoamino-phenyl)-ethyl]-carbamic acid tert-butyl ester; (R)-[1-(2-Morpholin-4-yl-ethylcarbamoyl)-2-(4-sulfoamino-phenyl)-ethyl]-carbamic acid tert-butyl ester; (S)-[1-Pentylcarbamoyl-2-(4-sulfoamino-phenyl)-ethyl]-carbamic acid tert-butyl ester; (S)-[4-(2-Hexanoylamino-2-methylcarbamoyl-ethyl)-phenyl]-sulfamic acid; (S)-{4-[2-Methylcarbamoyl-2-(toluene-4-sulfonylaniino)-ethyl]-phenyl}-sulfamic acid; (R)-{4-[2-Methylcarbamoyl-2-(3-phenyl-propionylamino)-ethyl]-phenyl}-sulfamic acid; (S)-{4-[2-Methylcarbamoyl-2-(3-phenyl-propionylamino)-ethyl]-phenyl}-sulfamic acid; (S)-[1-(2-Methoxy-ethylcarbamoyl)-2-(4-sulfoamino-phenyl)-ethyl]-carbamic acid tert-butyl ester; (S)-[1-(2-Ethoxy-ethylcarbamoyl)-2-(4-sulfoamino-phenyl)-ethyl]-carbamic acid tert-butyl ester; (S)-[1-(2-Ethylsulfanyl-ethylcarbamoyl)-2-(4-sulfoamino-phenyl)-ethyl]-carbamic acid tert-butyl ester; (S)-[1-(4-Phenyl-butylcarbamoyl)-2-(4-sulfoamino-phenyl)-ethyl]-carbamic acid tert-butyl ester; (S)-3-[2-tert-Butoxycarbonylamino-3-(4-sulfoamino-phenyl)-propionylamino]-propionic acid; (S)-{4-[2-(3-Benzyl-ureido)-2-methylcarbamoyl-ethyl]-phenyl}-sulfamnic acid; (S)-(4-{2-[3-(2-Methoxy-phenyl)-ureido]-2-methylcarbamoyl-ethyl}-phenyl)-sulfamic acid; (S)-[4-(2-Benzenesulfonylamino-2-methylcarbamoyl-ethyl)-phenyl]-sulfamic acid; (S)-{4-[2-(4-Methoxy-benzenesulfonylamino)-2-methylcarbamoyl-ethyl]-phenyl}-sulfamic acid; (S)-{4-[2-Methylcarbamoyl-2-(naphthalene-1-sulfonylamino)-ethyl]-phenyl}-sulfamic acid; (S)-[1-(Benzyl-methyl-carbamoyl)-2-(4-sulfoamino-phenyl)-ethyl]-carbamic acid tert-butyl ester; (S)-[1-(2-Methyl-5-phenyl-2H-pyrazol-3-ylcarbamoyl)-2-(4-sulfoamino-phenyl)-ethyl]-carbamic acid tert-butyl ester; (S)-[1-Phenylcarbamoyl-2-(4-sulfoamino-phenyl)-ethyl]-carbamic acid tert-butyl ester; (S)-[1-Dibenzylcarbamoyl-2-(4-sulfoamino-phenyl)-ethyl]-carbamic acid tert-butyl ester; (S)-4-[1-Methylcarbamoyl-2-(4-sulfoamino-phenyl)-ethylcarbamoyl]-piperidine-1-carboxylic acid tert-butyl ester; (S)-[4-(2-Benzoylamino-2-methylcarbamoyl-ethyl)-phenyl]-sulfamic acid; (S)-[1-Dimethylcarbamoyl-2-(4-sulfoamino-phenyl)-ethyl]-carbamic acid tert-butyl ester; (S)-(4-{2-Methylcarbamoyl-2-[(pyridine-3-carbonyl)-amino]-ethyl}-phenyl)-sulfamnic acid; (S)-[4-(2-Methylcarbamoyl-2-phenylacetylamino-ethyl)-phenyl]-sulfamic acid; (S)(4-{2-Methylcarbamoyl-2-[(naphthalene-1-carbonyl)-amino]-ethyl}-phenyl)-sulfamic acid; (S)-{4-[2-(Cyclopentanecarbonyl-amino)-2-methylcarbamoyl-ethyl]-phenyl}-sulfamic acid; (S)-(4-{2-Benzylcarbamoyl-2-[2-(4-propyl-phenyl)-acetylamino]-ethyl}-phenyl)-sulfamnic acid; (S)-(4-{2-[3-(3-Acetylsulfamoyl-pheny)-propionylamino]-2-methylcarbamoyl-ethyl}-phenyl)-sulfamic acid; (S)-{4-[2-Benzoylamino-2-(1-carbamoyl-2-(S)-phenyl-ethylcarbamoyl)-ethyl]-phenyl}-sulfamic acid; (S)-[1-[1-Carbamoyl-2-(4-hydroxy-phenyl)-ethylcarbamoyl]-2(S)-(4-sulfoamino-phenyl)-ethyl]-carbamic acid tert-butyl ester; [4-(2-{(tert-Butoxycarbonyl)[(4-methylphenyl)sulfonyl)-amino]ethyl}-phenyl]sulfamic acid; (4-{2-[Benzyl-(toluene-4-sulfonyl)-amino]-ethyl}-phenyl sulfamic acid; (4-{2-[(3-Methyl-but-2-enyl)-(toluene-4-sulfonyl)-amino]-ethyl}-phenyl)-sulfamic acid; (4-{2-[(3-Methyl-butyl)-(toluene-4-sulfonyl)-amino]-ethyl}-phenyl)-sulfamic acid; [[2-(4-Sulfoamino-phenyl)-ethyl]-(toluene-4-sulfonyl)-amino]-acetic acid ethyl ester; [[2-(4-Sulfoamino-phenyl)-ethyl]-(toluene-4-sulfonyl)-amino]-acetic acid; [4-(2-{[(4-Methylphenyl)sulfonyl][4-(sulfoamino)benzoyl]amino}ethyl)phenyl]sulfamic acid; (4-{2-[Benzoyl-(toluene-4-sulfonyl)-amino]-ethyl}-phenyl sulfamic acid; [4-(2-{tert-Butoxycarbonyl)[(3-fluoro-4-methylphenyl)sulfonyl]amino}ethyl)phenyl]sulfamic acid; [4-(2-{(tert-Butoxycarbonyl)[(3-fluorophenyl)sulfonyl]amino)ethyl}phenyl]sulfamic acid; [4-(2-{(tert-Butoxycarbonyl)[(2-fluorophenyl)sulfonyl]amino)ethyl}phenyl]sulfamic acid; {4-[2-(Toluene-4-sulfonlyamino)-ethyl]-phenyl}-sulfamic acid; [4-(2-Benzenesulfonylamino-ethyl)-phenyl]-sulfamic acid; [4-(2-Methanesulfonylamino-ethyl)-phenyl]-sulfamic acid; [4-(2-Methanesulfonylamino-ethyl)-phenyl]-sulfamic acid; {4-[2-(4-Methoxy-benzenesulfonylamino)-ethyl]-phenyl}-sulfamic acid; (S)-[4-(2-Dibenzylamino-2-methylcarbamoyl-ethyl-phenyl]-sulfamic acid; (S)-{4-[2-(Acetyl-benzyl-amino)-2-methylcarbamoyl-ethyl]-phenyl}-sulfamic acid; (S)-2-(Benzyl-tert-butoxycarbonyl-amino)-3-(4-sulfoamino-phenyl)-propionic acid methyl ester; (S)-Benzyl-[1-methylcarbamoyl-2-(4-sulfoamino-phenyl)-ethyl]-carbamic acid methyl ester; (S)-Benzyl-[1-methylcarbamoyl-2-(4-sulfoamino-phenyl)-ethyl]-carbamic acid tert-butyl ester; N-[(1,1-dimethylethoxy)carbonyl]-L-leucinyl-N-methyl-L-4-sulfoamino-phenylalaninamide; N-[(1,1-dimethylethoxy)carbonyl]-L-methionyl-N-methyl-L-4-sulfoamino-phenylalaninamide; N-[(1,1-dimethylethoxy)carbonyl]-L-phenylalanyl-N-methyl-L-4-sulfoamino-phenylalaninamide; N-[(1,1-dimethylethoxy)carbonyl]-L-tyrosinyl-N-methyl-L4-sulfoamino-phenylalaninamide; N-[(1,1-dimethylethoxy)carbonyl]-L-valinyl-N-methyl-L4-sulfoaino-phenylalaninamide; N-[(1,1-dimethylethoxy)carbonyl]-L-glutaminyl-N-methyl-L4-sulfoamino-phenylalaninamide; N-[(1,1-dimethylethoxy)carbonyl]-L-asparaginyl-N-methyl-L4-sulfoamino-phenylalaninamide; N-{1-[1-Pentylcarbamoyl-2-(4-sulfoamino-phenyl)-ethylcarbamoyl]-2-phenyl-ethyl}-succinamic acid; N-{1-L-[1-Pentylcarbamoyl-2-(4-sulfoamino-phenyl)-ethylcarbamoyl]-2-L-phenyl-ethyl}-carbamic acid tert-butyl ester; (S)-2-tert-Butoxycarbonylamino-3-(4-sulfoamino-phenyl)-propionic acid methyl ester; [2-(4-Sulfoamino-phenyl)-ethyl]-carbamic acid tert-butyl ester; [4-(2-Diphenylacetylamino-ethyl)-phenyl]-sulfamic acid; and (S)-[4-(3-Acetyl-1,2,2-trimethyl-5-oxo-imidazolidin-4-ylmethyl)-phenyl]-sulfamic acid; and N-[(1,1-dimethylethoxy)carbonyl]-L-prolinyl-N-methyl-L4-sulfoamino-phenylalaninamide.

19. A method of treating a protein tyrosine phosphatase (PTPase) mediated disorder comprising administering a compound of claim 1 to a subject in need thereof.

20. The method of claim 19, wherein the disorder is selected from the group consisting of atherosclerotic cardiovascular disease including peripheral vascular disease, coronary disease and cerebral vascular disease; heart failure; hypertension; diabetes (Type 1 or Type 2); skeletal muscle atrophy; osteoporosis; obesity; disorders of the gastrointestinal tract including inflammatory bowel disease and ulcer; wound healing and wrinkle repair/prevention; hair loss and cancer.

21. A pharmaceutical composition comprising:
a) safe and effective amount of a compound of claim 1; and
b) a pharmaceutically-acceptable carrier.
